(19) United States
(12) Patent Application Publication
(10) Pub. No.: US 2009/0176278 A1
(43) Pub. Date: Jul. 9, 2009

(54) SOLVENTS FOR MUTANT ENDOGLYCOCERAMIDASES WITH SYNTHETIC ACTIVITY

(75) Inventors: Shawn Defrees, North Wales, PA (US); Xiaoping Tang, North Wales, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: NEOSE TECHNOLOGIES, INC., HORSHAM, PA (US)

(21) Appl. No.: 12/095,721

(22) PCT Filed: Dec. 1, 2006
(86) PCT No.: PCT/US06/46023

§ 371 Date: Jun. 23, 2008

Related U.S. Application Data
(60) Provisional application No. 60/742,055, filed on Dec. 1, 2005.

(51) Int. Cl. C12P 19/00; C12N 9/24

(52) U.S. Cl. 435/72; 435/200

(57) ABSTRACT

The present invention provides reaction mixtures comprising a solvent having at least one of an alkoxy ether and/or a polyhydric alcohol for use in reactions with a mutant endoglycoceramidase having enhanced synthetic activity.

### CROSS-REFERENCE TO RELATED APPLICATIONS

This application claims the benefit of U.S. Provisional Application No. 60/742,055, filed on Dec. 1, 2005, the entire contents of which is hereby incorporated herein by reference for all purposes.

### FIELD OF THE INVENTION

The present invention relates to the field of synthesis of saccharides, particularly those of use in preparing glycolipids, e.g., glycosphingolipids. More specifically, the invention provides reaction mixtures comprising a solvent having at least one of an alkoxy ether and/or a polyhydric alcohol for use with a mutant endoglycoceramidase, which has a synthetic activity that can be used to catalyze the formation of the glycosidic linkage between a monosaccharide or oligosaccharide and an aglycone to form various glycolipids.

### BACKGROUND OF THE INVENTION

Glycolipids, a group of amphipathic compounds that structurally consist of a sugar chain (monosaccharide or oligosaccharide) bound to an aglycone, are important cellular membrane components known to participate in various cellular events mediating physiological processes such as the cell-cell recognition, antigenicity, and cell growth regulation (Hakomori, Annu. Rev. Biochem., 50: 733-764, 1981; Makita and Taniguchi, Glycolipid (Wiegandt, ed.) pp 59-82, Elsevier Scientific Publishing Co., New York, 1985). Because there are no known enzymes that can universally transfer a saccharyl residue to a an aglycone (e.g., ceramide or sphingosine), synthesis of glycolipids usually requires a multi-step complex process that has the disadvantages of high cost and low yield.

Endoglycoceramidase (EC3.2.1.123), an enzyme first isolated from the Actinomycetes of Rhodococcus strain (Horibata, J. Biol. Chem. May 2004 10.1094/jbc.M401-460200; Ito and Yamagata, J. Biol. Chem., 261: 14278-14282, 1986), hydrolyzes the glycoside linkage between the sugar chain and the ceramide in glycolipids to produce intact monosaccharide or oligosaccharide and ceramide. To this date, several more endoglycoceramidases have been isolated and characterized (see e.g., Li et al., Biochem. Biophy. Res. Comm., 149: 167-172, 1987; Ito and Yamagata, J. Biol. Chem., 264: 9510-9519, 1989; Zhou et al., J. Biol. Chem., 264: 12272-12277, 1989; Ashida et al., Eur. J. Biochem., 205: 729-735, 1992; Izu et al., J. Biol. Chem., 272: 19846-19850, 1997; Horibata et al., J. Biol. Chem., 275:31297-31304, 2000; Sakaguchi et al., J. Biochem., 128: 145-152, 2000; and U.S. Pat. No. 5,795,765). The active site of endoglycoceramidases has also been described by Sakaguchi et al., Biochem. Biophy. Res. Comm., 260: 89-93, 1999, as including a three amino acid segment of Asn-Glu-Pro, among which the Glu residue appears to be the most important to the enzymatic activity.

Endoglycoceramidases are also known to possess an additional transglycosylation activity, which is much weaker than the hydrolytic activity (Li et al., J. Biol. Chem., 266:10723-10726, 1991; Ashida et al., Arch. Biochem. Biophy., 305:559-562, 1993; Horibata et al., J. Biochem., 130:263-268, 2001). This transglycosylation activity has been exploited to synthesize glycolipids (see, PCT/US05/19451) The present invention provides improved reaction mixtures for efficient use of such a synthetic mutant endoglycoceramidase (“endoglycoceramide synthase”).

### BRIEF SUMMARY OF THE INVENTION

The present invention provides reaction mixtures comprising: a) a solvent comprising at least one of a polyhydric alcohol or an alkoxy ether; and b) a mutant endoglycoceramidase having a modified nucleophilic carboxylate amino acid residue, wherein the nucleophilic carboxylate residue resides within a (Ile/Met/Leu/Phe/Val)-(Leu/Met/Ile/Val)-(Gly/Ser/Thr)-(Glu/Asp)-(Phe/Thr/Met/Leu)-(Gly/Leu/Phe) sequence (motif E or SEQ ID NO:5). In some embodiments, the alkoxy ether is the lower alkoxy diether 1,2-dimethoxymethane.

In some embodiments, the alkoxy ether is a lower alkoxy ether. In some embodiments, the alkoxy ether is a diether. In some embodiments, the alkoxy ether is a glyme. In some embodiments, the alkoxy ether is 1,2 dimethoxyethane. In some embodiments, the polyhydric alcohol is glycerol.

In some embodiments, the alkoxy ether is one or more selected from the group consisting of dimethoxymethane, 1,2-dimethoxyethane, 1,1 dimethoxyethane, ethylene glycol diethyl ether, 1,1-diethoxyethane, propylene glycol diethyl ether, di(propylene glycol) dimethyl ether, ethylene glycol dipropyl ether, diethylene glycol butyl ether, diethylene glycol dibutyl ether, diethylene glycol monoethyl ether, diethylene glycol diethyl ether, diethylene glycol methyl ether, diethylene glycol dimethyl ether, diethylene glycol monopropyl ether, ethylene glycol butyl ether, 1,2-dimethoxyethene, 1,1-dimethoxyethene, diethyl ether and tert-butyl methyl ether.

In some embodiments, the polyhydric alcohol is one or more selected from the group consisting of glycerol, diglycerol, ethylene glycol, diethylene glycol, triethylene glycol, propylene glycol, dipropylene glycol, trimethylolpropane, pentaerythritol, and 1,3-butanediol, sorbitol, mannitol, xylitol, erythritol, maltitol, lactitol, polyethylene glycol, polypropylene glycol, hexanediol, pentanediol, hexanetriol, and thiodiglycol.

The reaction mixtures usually will also contain a donor substrate a saccharide moiety and an acceptor substrate. In some embodiments, the donor substrate is an α modified glycosyl donor of anomeric configuration opposite the natural glycosidic linkage. In some embodiments, the donor substrate is a glycosyl fluoride.

In some embodiments, the acceptor substrate is an aglycone of Formulas Ia, Ib, II or III. In some embodiments, the acceptor substrate is a sphingosine or a sphingosine analog. In some embodiments, the sphingosine is selected from the group consisting of from D-erytbro-sphingosine, D-erythro-sphinganine, L-threo-sphingosine, L-threo-dihydrosphingosine, D-erythro-phytosphingosine, N-ocatanoyl-D-erythro-sphingosine. In some embodiments, the acceptor substrate is a ceramide.

In a another aspect, the invention provides methods of synthesizing a glycolipid, the method comprising, contacting in a reaction mixture comprising at least one of an alkoxy ether and a polyhydric alcohol, a donor substrate a saccharide moiety and an acceptor substrate, with a mutant endoglycoceramidase having a modified nucleophilic carboxylate amino acid residue, wherein the nucleophilic carboxylate residue resides within a (Ile/Met/Leu/Phe/Val)-(Leu/Met/Ile/Val)-(Gly/Ser/Thr)-(Glu/Asp)-(Phe/Thr/Met/Leu)-(Gly/Leu/Phe) sequence (motif E or SEQ ID NO:5) of a corresponding wild-type endoglycoceramidase, under conditions wherein the endoglycoceramidase catalyzes the transfer of a saccharide moiety from a donor substrate to an acceptor substrate, thereby producing the glycolipid.

The embodiments for the methods can be the same as set forth for the reaction mixtures. In some embodiments, the reaction mixtures are produced on a large scale. In some embodiments, the methods are carried out on a large scale.

Other objects, aspects and advantages of the invention will be apparent from the detailed description that follows.

### DEFINITIONS

A “glycolipid” is a covalent conjugate between a glycosyl moiety and a substrate for a mutant endoglycoceramidase of the invention, such as an aglycone. An exemplary “glycolipid” is a covalent conjugate, between a glycosyl moiety and an aglycone, formed by a mutant endoglycoceramidase of the invention. The term “glycolipid” encompasses all glycosphingolipids, which are a group of amphipathic compounds that structurally consist of a sugar chain moiety (monosaccharide, oligosaccharide, or derivatives thereof) and an aglycone (i.e., a ceramide, a sphingosine, or a sphingosine analog). This term encompasses both cerebrosides and gangliosides. In certain embodiments, a glycolipid is an aglycone (non-carbohydrate alcohol (OH) or (SH)) conjugated to a non-reducing sugar and a non-glycoside.

An “aglycone,” as referred to herein, is an acceptor substrate onto which a mutant endoglycoceramidase of the invention transfers glycosyl moiety from a glycosyl donor that is a substrate for said glycosyl donor. A glycosyl donor may be an activated or non-activated saccharide. An exemplary aglycone is a heteroalkyl moiety, which has the structure of, e.g., Formula Ia, Formula Ib or Formula II as shown below:

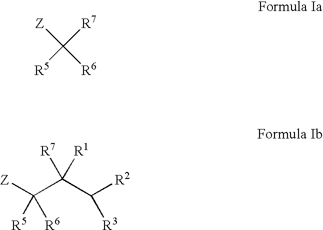

In Formula Ia and Formula Ib, the symbol Z represents OH, SH, or NR⁴R^{4′}. R¹ and R² are members independently selected from NHR⁴, SR⁴, OR⁴, OCOR⁴, OC(O)NHR⁴, NHC(O)OR⁴, OS(O)₂OR⁴, C(O)R⁴, NHC(O)R⁴, detectable labels, and targeting moieties. The symbols R³, R⁴ and R^{4′}, R⁵, R⁶ and R⁷ each are members independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl.

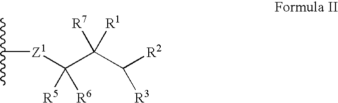

In Formula II, Z¹ is a member selected from O, S, and NR⁴; R¹ and R² are members independently selected from NHR⁴, SR⁴, OR⁴, OCOR⁴, OC(O)NHR⁴, NHC(O)OR⁴, OS(O)₂OR⁴, C(O)R⁴, NHC(O)R⁴, detectable labels, and targeting moieties. The symbols R³, R⁴, R⁵, R⁶ and R⁷ each are members independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl. Formula II is representative of certain embodiments wherein the aglycone portion is conjugated to a further substrate component, for example, a leaving group or a solid support.

The term “ceramide,” as used herein, encompasses all ceramides and sphingosine as conventionally defined. See, for example, Berg, et al, Biochemistry, 2002, 5th ed., W.H. Freeman and Co.

The term “sphingosine analog” refers to lipid moieties that are chemically similar to sphingosine, but are modified at the polar head and/or the hydrophobic carbon chain. Sphingolipid analog moieties useful as acceptor substrates in the present methods include, but are not limited to, those described in co-pending patent applications PCT/US2004/006904 (which claims priority to U.S. Provisional Patent Application No. 60/452,796); U.S. patent application Ser. No. 10/487,841; U.S. patent application Ser. No. 10/485,892; 10/485,195, and PCT/US2005/040195 (which claims priority to U.S. Provisional Patent Application No. 60/626,678); the disclosures of each of which are hereby incorporated herein by reference in their entirety for all purposes.

In general, the sphingosine analogs described in the above-referenced applications are those compounds having the formula:

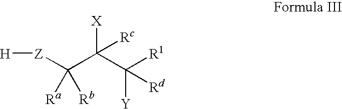

wherein Z is a member selected from O, S, C(R²)₂ and NR²; X is a member selected from H, —OR³, —NR³R⁴, —SR³, and —CHR³R⁴; R¹, R², R³ and R⁴ are members independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl, —C(=M)R⁵, —C(=M)-Z¹-R⁵, —SO₂R⁵, and —SO₃; wherein M and Z¹ are members independently selected from O, NR⁶ or S; Y is a member selected from H, —OR⁷, —SR⁷, —NR⁷R⁸, substituted or unsubstituted alkyl, substituted or unsubstituted heteroalkyl, substituted or unsubstituted aryl, substituted or unsubstituted heteroaryl, and substituted or unsubstituted heterocycloalkyl, wherein R⁵, R⁶, R⁷ and R⁸ are members independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl; and R^{a}, R^{b}, R^{c} and R^{d} are each independently H, substituted or unsubstituted alkyl, substituted or unsubstituted heteroalkyl, substituted or unsubstituted aryl, substituted or unsubstituted heteroaryl, substituted or unsubstituted heterocycloalkyl.

An “acceptor substrate” for a wild-type endoglycoceramidase or a mutant endoglycoceramidase, is any aglycone moiety that can act as an acceptor for a particular endoglycoceramidase. When the acceptor substrate is contacted with the corresponding endoglycoceramidase and sugar donor substrate, and other necessary reaction mixture components, and the reaction mixture is incubated for a sufficient period of time, the endoglycoceramidase transfers sugar residues from the sugar donor substrate to the acceptor substrate. The acceptor substrate can vary for different types of a particular endoglycoceramidase. Accordingly, the term “acceptor substrate” is taken in context with the particular endoglycoceramidase or mutant endoglycoceramidase of interest for a particular application. Acceptor substrates for endoglycoceramidases and mutant endoglycoceramidases are described herein.

A “donor substrate” for wild-type and mutant endoglycoceramidases includes any activated glycosyl derivatives of anomeric configuration opposite the natural glycosidic linkage. The enzymes of the invention are used to couple α-modified or β-modified glycosyl donors, usually α-modified glycosyl donors, with glycoside acceptors. Preferred donor molecules are glycosyl fluorides, although donors with other groups which are reasonably small and which function as relatively good leaving groups can also be used. Examples of other glycosyl donor molecules include glycosyl halides (e.g., chlorides, bromides, iodides, fluorides), acetates, mesylates, propionates, pivaloates, and glycosyl molecules modified with substituted phenols. Among the α-modified or β-modified glycosyl donors, α-galactosyl, α-mannosyl, α-glucosyl, α-fucosyl, α-xylosyl, α-sialyl, α-N-acetylglucosaminyl, α-N-acetylgalactosaminyl, β-galactosyl, β-mannosyl, β-glucosyl, β-fucosyl, β-xylosyl, β-sialyl, β-N-acetylglucosaminyl and β-N-acetylgalactosaminyl are most preferred. The donor molecules can be monosaccharides, or may themselves contain multiple sugar moieties (oligosaccharides). Donor substrates of use in the particular methods include those described in U.S. Pat. Nos. 6,284,494; 6,204,029; 5,952,203; and 5,716,812, the disclosures of which are hereby incorporated herein by reference in their entirety for all purposes.

The term “contacting” is used herein interchangeably with the following: combined with, added to, mixed with, passed over, incubated with, flowed over, etc.

“Endoglycoceramidase,” as used herein, refers to an enzyme that in its native or wild-type version has a primary activity of cleaving the glycosidic linkage between a monosaccharide or an oligosaccharide and a ceramide (or sphingosine) of an acidic or neutral glycolipid, producing intact monosaccharide or oligosaccharide and ceramide (Registry number: EC 3.2.1.123). The wild-type version of this enzyme may also have a secondary activity of catalyzing the formation of the glycosidic linkage between a monosaccharide or oligosaccharide and an aglycone (i.e., a ceramide or a sphingosine) to form various glycolipids. Wild-type endoglycoceramidases have at least two identifiable conserved motifs, including an acid-base region (Val-X₁-(Ala/Gly)-(Tyr/Phe)-(Asp/Glu)-(Leu/Ile)-X₂-Asn-Glu-Pro-X₃-X₄-Gly or motif B or SEQ ID NO:2), and a nucleophilic region ((Ile/Met/Leu/Phe/Val)-(Leu/Met/Ile/Val)-(Gly/Ser/Thr)-Glu-(Phe/Thr/Met/Leu)-(Gly/Leu/Phe or motif D or SEQ ID NO:4).

The terms “mutated” or “modified” as used in the context of altering the structure or enzymatic activity of a wild-type endoglycoceramidase, refers to the deletion, insertion, or substitution of any nucleotide or amino acid residue, by chemical, enzymatic, or any other means, in a polynucleotide sequence encoding an endoglycoceramidase or the amino acid sequence of a wild-type endoglycoceramidase, respectively, such that the amino acid sequence of the resulting endoglycoceramidase is altered at one or more amino acid residues. The site for such an activity-altering mutation may be located anywhere in the enzyme, including within the active site of the endoglycoceramidase, particularly involving the glutamic acid residue of the Asn-Glu-Pro subsequence of the acid-base sequence region. An artisan of ordinary skill will readily locate this Glu residue, for example, at position 233 in SEQ ID NO:7 and at position 224 in SEQ ID NO:9. Other examples of Glu residues that, once mutated, can alter the enzymatic activity of an endoglycoceramidase include a carboxylate (i.e., Glu or Asp) nucleophilic Glu/Asp residue (bolded) in the (Ile/Met/Leu/Phe/Val)-(Leu/Met/Ile/Val)-(Gly/Ser/Thr)-Glu/Asp-(Phe/Thr/Met/Leu)-(Gly/Leu/Phe) motif of a corresponding wild-type endoglycoceramidase.

A “mutant endoglycoceramidase” or “modified endoglycoceramidase” used in the methods of this invention thus comprises at least one mutated or modified amino acid residue, as described in International Application No. PCT/US05/19451 and U.S. Provisional Application Nos. 60/666,765; 60/626,791, 60/576,316, the disclosures of each of which are hereby incorporated herein by reference in their entirety for all purposes. The wild-type endoglycoceramidase whose coding sequence is modified to generate a mutant endoglycoceramidase is referred to in this application as “the corresponding native or wild-type endoglycoceramidase.” One exemplary mutant endoglycoceramidase of the invention includes the deletion or substitution of a nucleophilic carboxylate Glu/Asp residue (bolded) in the (Ile/Met/Leu/Phe/Val)-(Leu/Met/Ile/Val)-(Gly/Ser/Thr)-Glu/Asp-(Phe/Thr/Met/Leu)-(Gly/Leu/Phe) motif of a corresponding wild-type endoglycoceramidase. One exemplary mutant endoglycoceramidase of the invention includes a mutation within the active site, e.g., the deletion or substitution of the Glu residue within the Asn-Glu-Pro subsequence of the acid-base sequence region. The mutant endoglycoceramidase exhibits an altered enzymatic activity, e.g., an enhanced glycolipid synthetic activity, in comparison with its wild-type counterpart. A mutant endoglycoceramidase that has demonstrated an increased glycolipid synthetic activity is also called an “endoglycoceramide synthase.”

The term “acid-base sequence region” refers to a conserved Val-X₁-(Ala/Gly)-(Tyr/Phe)-(Asp/Glu)-(Leu/Ile)-X₂-Asn-Glu-Pro-X₃-X₄-Gly sequence (SEQ ID NO:2) in a corresponding wild-type endoglycoceramidase which includes a conserved Asn-Glu-Pro subsequence. The acid-base glutamic acid residue is located within the conserved Asn-Glu-Pro subsequence, for example, at position 233 in Rhodococcus sp. M-777; position 224 in Rhodococcus sp. C9; position 229 in Propionibacterium acnes EGCa; position 248 in Propionibacterium acnes EGCb; position 238 in Cyanea nozakii; at position 229 in Hydra magnipapillata; at postion 234 in Dictyostelium; at position 214 in Schistosoma; at position 241 in Leptospira interrogans; at position 272 of Streptomyces; and at position 247 of Neurosporassa (see, FIG. 14). The conserved sequence encoding a three-amino acid segment Asn-Glu-Pro was previously identified within the active site of endoglycoceramidases, and the Glu residue within the segment was thought to be connected to the hydrolytic activity of the endoglycoceramidase (Sakaguchi et al., Biochem. Biophys. Res. Commun., 1999, 260: 89-93).

The term “nucleophilic residue” or “nucleophilic motif” refers to the carboxylate amino acid residue within the (Ile/Met/Leu/Phe/Val)-(Leu/Met/Ile/Val)-(Gly/Ser/Thr)-(Asp/Glu)-(Phe/Thr/Met/Leu)-(Gly/Leu/Phe) motif (SEQ ID NO:5) of a corresponding wild-type endoglycoceramidase. The nucleophilic residue can be a glutamate or an aspartate, usually a glutamate. A nucleophilic glutamic acid residue is located, for example, at position 351 in Rhodococcus sp. M-777; position 343 in Rhodococcus sp. C9; position 342 in Propionibacterium acnes EGCa; position 360 in Propionibacterium acnes EGCb; position 361 in Cyanea nozakii; and at position 349 in Hydra magnipapillata; at position 354 in Dictyostelium; at position 351 in Schistosoma; at position 461 in Leptospira interroganss; at position 391 of Streptomyces; and at position 498 of Neurosporassa (see, FIG. 14).

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid (i.e., hydrophobic, hydrophilic, positively charged, neutral, negatively charged). Exemplified hydrophobic amino acids include valine, leucine, isoleucine, methionine, phenylalanine, and tryptophan. Exemplified aromatic amino acids include phenylalanine, tyrosine and tryptophan. Exemplified aliphatic amino acids include serine and threonine. Exemplified basic aminoacids include lysine, arginine and histidine. Exemplified amino acids with carboxylate side-chains include aspartate and glutamate. Exemplified amino acids with carboxamide side chains include asparagines and glutamine. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

The term “alkyl,” by itself or as part of another substituent, means, unless otherwise stated, a straight or branched chain, or cyclic hydrocarbon radical, or combination thereof, which may be fully saturated, mono- or polyunsaturated and can include mono-, di- and multivalent radicals, having the number of carbon atoms designated (i.e. C₁-C₁₀ means one to ten carbons). Examples of saturated alkyl radicals include, but are not limited to, groups such as methyl, methylene, ethyl, ethylene, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, includes “alkylene” and those derivatives of alkyl defined in more detail below, such as “heteroalkyl.” Alkyl groups, which are limited to hydrocarbon groups, are termed “homoalkyl.”

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. The term “alkoxy” refers to a heteroalkyl that contains one or more oxygen heteroatoms. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written.

Each of the above terms (e.g., “alkyl” and “heteroalkyl”) are meant to include both substituted and unsubstituted forms of the indicated radical. Preferred substituents for each type of radical are provided below.

A “lower alkyl” or “lower alkoxy” refers to a chemical compound or chemical moiety having 12 or less carbon atoms, usually less then 10 or 8 carbon atoms, more usually less than 6 carbon atoms.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)=NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Similar to the substituents described for the alkyl radical, substituents for the aryl and heteroaryl groups are varied and are selected from, for example: halogen, OR′, —NR′R″, —SR′, -halogen, —SiR′R″R′″, OC(O)R′, —C(O)R′, CO2R′, —CONR′R″, —OC(O)NR′R″, —NR″C(O)R′, NR′C(O)NR″R′″, —NR″C(O)₂R′, NR—C(NR′R″R′″)═NR″″, NRC(NR′R″)═NR′″, —S(O)R′, —S(O)₂R′, —S(O)₂NR′R″, NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 40. Alternatively, two of the substituents on adjacent atoms of the aryl or heteroaryl ring may optionally be replaced with a substituent of the formula -A(CH₂)ᵣB—, wherein A and B are independently —CRR′—, —O—, —NR—, —S—, —S(O)—, S(O)₂, —S(O)₂NR′— or a single bond, and r is an integer of from 1 to 40. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 40, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₄₀)alkyl.

The term “glyme” refers to a glycol ether. A glyme can be an end-capped polyethylene glycol or a capped polypropylene glycol, for example a polyglycol dimethyl ether. The end capping of the glycol chain can be independently, one or more methyl-, ethyl-, propyl-, isopropyl-, butyl-, tert-butyl-, allyl-, vinyl- or another lower alkyl moiety. Exemplified glymes include without limitation, monoglymes (e.g., 1,2-dimethoxy ethane, ethylene glycol dimethyl ether, dimethyl glycol), diglymes (e.g., dithylene glycol dimethyl ether, diethylene glycol dibutyl ether), triglymes (e.g., triethylene glycol dimethyl ether), and tetraglymes (e.g., tetraethylene glycol dimethyl ether).

The term “polyhydric alcohol” is intended to cover any organic multiple hydroxy-compound of a predominantly alcholic function. Non-limiting examples include straight or branched chain diols including propylene or butylene glycol and triols including glycerol. Polyhydric alcohols can be of a mixed type, e.g., with primary and secondary—OH groups. Glycerol is an example of a mixed type polyhydric alcohol. Polyhydric alcohols of particular interests are compatible both with water and hydrophobic substances. Although usually based on straight or branched alkyl chains, the polyhydric alcohols can also be carbocyclic, alicyclic, heterocyclic, or aromatic.

The symbol [symbol] , whether utilized as a bond or displayed perpendicular to a bond indicates the point at which the displayed moiety is attached to the remainder of the molecule, solid support, etc.

The term “increase,” as used herein, refers to a detectable positive change in quantity of a parameter when compared to a standard. The level of this positive change, for example, in the synthetic activity of a mutant endoglycoceramidase from its corresponding wild-type endoglycoceramidase, is preferably at least 10% or 20%, and more preferably at least 30%, 40%, 50%, 60% or 80%, and most preferably at least 100%.

The term “reduce” or “decrease” is defined as a detectable negative change in quantity of a parameter when compared to a standard. The level of this negative change, for example, in the hydrolytic activity of a mutant endoglycoceramidase from its corresponding wild-type endoglycoceramidase, is preferably at least 10% or 20%, and more preferably at least 30%, 40%, 50%, 60%, 80%, 90%, and most preferably at least 100%.

The phrase “large-scale” synthesis refers to a reaction that is carried out on at least a 0.1 gram scale. A large-scale synthesis produces at least 100 mg (0.1 g) of glycolipid product.

The terms “yield” or “isolated product yield” interchangeably refer to the number of moles of isolated product divided by the number of moles of limiting starting reagent and then multiplied by 100%. The number of moles of isolated product is determined by dividing the mass of the isolated product by the molecular weight of the product. The number of moles of limiting starting reagent is determined by dividing the mass of the limiting reagent by the molecular weight of the limiting reagent.

### BRIEF DESCRIPTION OF THE DRAWINGS

FIG. 1 sets forth compounds that can be made using the enzymes of the invention.

FIG. 2 sets forth compounds that can be made using the enzymes of the invention.

FIG. 3 sets forth compounds that can be made using the enzymes of the invention.

FIG. 4 sets forth compounds that can be made using the enzymes of the invention.

FIG. 5 sets forth compounds that can be made using the enzymes of the invention.

FIG. 6 sets forth compounds that can be made using the enzymes of the invention.

FIG. 7 sets forth compounds that can be made using the enzymes of the invention.

FIG. 8 sets forth compounds that can be made using the enzymes of the invention.

FIG. 9 sets forth compounds that can be made using the enzymes of the invention.

FIG. 10 sets forth compounds that can be made using the enzymes of the invention.

FIG. 11 sets forth compounds that can be made using the enzymes of the invention.

FIG. 12 sets forth compounds that can be made using the enzymes of the invention.

FIG. 13 sets forth compounds that can be made using the enzymes of the invention.

FIG. 14A-C illustrates an amino acid sequence alignment of wild-type endoglycoceramidases from Rhodococcus, Propionibacterium, Cyanea, and Hydra.

### DETAILED DESCRIPTION

### Introduction

Glycolipids, each consisting of a saccharide moiety and a heteroalkyl moiety, e.g., Formula Ia, Formula Ib, Formula II or Formula III, are important constituents of cellular membranes. With their diverse sugar groups extruding outward from the membrane surface, glycolipids mediate cell growth and differentiation, recognize hormones and bacterial toxins, and determine antigenicity; some are recognized as tumor-associated antigens (Hakomori, Annu. Rev. Biochem., 50:733-764, 1981; Marcus, Mol. Immmol. 21:1083-1091, 1984). The present invention discloses solvents of particular use with mutant endoglyceramidases having synthetic activity and methods for producing glycolipids having a saccharyl moiety of virtually any structure employing such solvents.

### Methods for Synthesizing a Glycolipid Using Mutant Endoglycoceramidases

The invention provides improved methods of synthesizing a glycolipid or aglycone. The methods include using a reaction mixture comprising a solvent comprising at least one of an alkoxy ether and a polyhydric alcohol and contacting a glycosyl donor comprising a glycosyl group, and an aglycone with a mutant endoglycoceramidase of the invention under conditions appropriate to transfer said glycosyl group to said aglycone.

In one aspect, the invention provides a method of synthesizing a glycolipid or aglycone in a reaction mixture comprising a solvent comprising at least one of an alkoxy ether or a polyhydric alcohol, the method comprising contacting a donor substrate comprising a saccharide moiety and an acceptor substrate with a mutant endoglycoceramidase having a modified nucleophilic carboxylate (i.e., Glu or Asp) residue, wherein the nucleophilic Glu/Asp resides within a (Ile/Met/Leu/Phe/Val)-(Leu/Met/Ile/Val)-(Gly/Ser/Thr)-(Glu/Asp)-(Phe/Thr/Met/Leu)-(Gly/Leu/Phe) sequence of a corresponding wild-type endoglycoceramidase, under conditions wherein the endoglycoceramidase catalyzes the transfer of a saccharide moiety from a donor substrate to an acceptor substrate, thereby producing the glycolipid or aglycone.

In a further aspect, the invention provides a method of synthesizing a glycolipid or aglycone in a reaction mixture comprising a solvent comprising at least one of an alkoxy ether or a polyhydric alcohol, the method comprising, contacting a donor substrate comprising a saccharide moiety and an acceptor substrate with a mutant endoglycoceramidase having a modified Glu residue within the subsequence of Asn-Glu-Pro, wherein the subsequence resides within the acid-base sequence region of Val-X₁-(Ala/Gly)-(Tyr/Phe)-(Asp/Glu)-(Leu/Ile)-X₂-Asn-Glu-Pro-X₃-X₄-Gly sequence in the corresponding wild-type protein, under conditions wherein the endoglycoceramidase catalyzes the transfer of a saccharide moiety from a donor substrate to an acceptor substrate, thereby producing the glycolipid or aglycone.

In carrying out the methods of glycolipid synthesis, one or both of the nucleophilic carboxylate amino acid residue (i.e., a Glu or an Asp) and/or acid-base sequence region Glu residues of a corresponding wild-type endoglycoceramidase can be deleted or replaced with another chemical moiety that retains the integral structure of the protein such that the mutant enzyme has synthetic activity. For example, one or more of the nucleophilic carboxylate amino acid residues (Glu or Asp) and/or acid-base sequence region Glu residues can be replaced with an L-amino acid residue other than Glu or Asp, a D-amino acid residue (including a D-Glu or a D-Asp), an unnatural amino acid, an amino acid analog, an amino acid mimetic, and the like. Usually, the one or more carboxylate amino acid residues (Glu or Asp) are substituted with another L-amino acid other than Glu or Asp, for example, Gly; Ala, Ser, Asp, Asn, Glu, Gln, Cys, Thr, Ile, Leu or Val.

In one embodiment, the mutant enzymes of the invention converts at least about 50% of the starting materials, based upon the limiting reagent, to a desired glycolipid, more preferably, at least about 60%, 70%, 80% or 90%. In another preferred embodiment, the conversion of the limiting reagent to glycolipid is virtually quantitative, affording a conversion that is at least about 90%, and more preferably, at least about 92%, 94%, 96%, 98% and even more preferably, at least about 99%.

In another exemplary embodiment, the glycosyl donor and the acceptor substrate (i.e., aglycone) are present in a molar ratio range of from about 2:1 to about 1:2, for example, about 2:1, 1.5:1, 1.4:1, 1.1:1, 1:1, 1:1.1, 1:1.4, 1:1.5, 1:2 (donor:acceptor), and the enzyme of the invention, acting catalytically, converts the two reagents to a glycolipid in at least about 40% yield, more preferably at least about 45%, 50%, 55%, 60%, 65%, 70%, 75%, 80% or more. In a further exemplary embodiment, the conversion is essentially quantitative as discussed above. Yield can be measured using any methods known in the art. In some embodiments, yield is measured using HPLC.

In one embodiment, the synthesized glycolipid is an aglycone (non-carbohydrate alcohol (OH) or (SH)) conjugated to a non-reducing sugar and a non-glycoside.

### Production of Glycolipids

Wild-type and mutant endoglycoceramidase polypeptides can be used to make glycolipid products in in vitro reaction mixtures.

### Reaction Mixtures for In Vitro Reactions

The wild-type and mutant endoglycoceramidase polypeptides can be used to make sialylated products in in vitro reaction mixtures.

The reaction mixtures of the invention generally include a mutant endoglycoceramidase of the invention, a solvent containing at least one of an alkoxy ether and a polyhydric alcohol, a donor substrate, an acceptor substrate, and appropriate reaction buffers. The reaction medium optionally can comprise solubilizing detergents (e.g., Triton or SDS) and additional organic solvents such as methanol or ethanol, if necessary. The reaction mixture can also include divalent metal cations (Mg²⁺, Mn²⁺). The enzymes can be utilized free in solution or can be bound to a support such as a polymer. The reaction mixture is thus substantially homogeneous at the beginning, although some precipitate can form during the reaction.

In some embodiments, the alkoxy ether is a lower alkoxy ether. The alkoxy ether can be a monoether, a diether, and in certain embodiments, a polymer. In some embodiments, the alkoxy ether is a glyme.

In some embodiments, the alkoxy ether is one or more selected from the group consisting of dimethoxymethane, 1,2-dimethoxyethane, 1,1 dimethoxyethane, ethylene glycol diethyl ether, 1,1-diethoxyethane, propylene glycol diethyl ether, di(propylene glycol) dimethyl ether, ethylene glycol dipropyl ether, diethylene glycol butyl ether, diethylene glycol dibutyl ether, diethylene glycol monoethyl ether, diethylene glycol diethyl ether, diethylene glycol methyl ether, diethylene glycol dimethyl ether, diethylene glycol monopropyl ether, ethylene glycol butyl ether, 1,2-dimethoxyethene, 1,1-dimethoxyethene, diethyl ether and tert-butyl methyl ether. In some embodiments, the alkoxy ether is 1,2-dimethoxyethane.

The final concentration of alkoxy ether included in the reaction mixture is sufficient for enzymatic activity of the endoglycoceramidase. An appropriate concentration can be readily determined by those of skill in the art, usually starting at a lower concentration and then incrementally increasing or decreasing, as necessary, until a desired level of enzymatic activity is achieved. The final concentration of alkoxy ether included in the reaction mixture can be in the range of about 10% to about 20% (v/v), for example about 12% to about 18%, or about 14% to about 16%, for example, 10%, 11%, 12%, 13%, 14%, 15%, 16%, 17%, 18%, 19%, or 20%.

In some embodiments, the polyhydric alcohol is one or more selected from the group consisting of glycerol, diglycerol, ethylene glycol, diethylene glycol, triethylene glycol, propylene glycol, dipropylene glycol, trimethylolpropane, pentaerythritol, and 1,3-butanediol, sorbitol, mannitol, xylitol, erythritol, maltitol, lactitol, polyethylene glycol, polypropylene glycol, hexanediol, pentanediol, hexanetriol, and thiodiglycol. In some embodiments, the polyhydric alcohol is glycerol.

The final concentration of polyhydric alcohol included in the reaction mixture is sufficient for enzymatic activity of the endoglycoceramidase. An appropriate concentration can be readily determined by those of skill in the art, usually starting at a lower concentration and then incrementally increasing or decreasing, as necessary, until a desired level of enzymatic activity is achieved. The final concentration of polyhydric alcohol included in the reaction mixture can be in the range of about 0% to about 25% (v/v). When present, the polyhydric alcohol can be included at a final concentration (v/v) of about 8% to about 25%, or about 9% to about 13%, for example, 8%, 9%, 10%, 11%, 12%, 13%, 14%, 15%, 16%, 17%, 18%, 19%, 20% 21%, 22%, 23%, 24% or 25%. In some embodiments, the alkoxy ether and polyhydric alcohol are included in the reaction mixture at a concentration ratio (v:v) of about 1.5:1, 1.4:1, 1.3:1, 1.2:1, 1.1:1 (alkoxy ether:polyhydric alcohol). In some embodiments, the reaction mixtures are free of detergent.

The in vitro reaction mixtures can include permeabilized microorganisms comprising the wild-type or mutant endoglycoceramidase polypeptides, partially purified endoglycoceramidase polypeptides, or purified endoglycoceramidase polypeptides. For in vitro reactions, the recombinant wild-type or mutant endoglycoceramidase proteins, acceptor substrates, donor substrates and other reaction mixture ingredients are combined by admixture in an aqueous reaction medium. Additional glycosyltransferases can be used in combination with the endoglycoceramidase polypeptides, depending on the desired glycolipid end product. The medium generally has a pH value of about 4.5 to about 8.5, for example, about 5.0 to about 7.5; or about 5.0 to about 6.0, or about 4.5 to about 5.5. The selection of a medium is based on the ability of the medium to maintain pH value at the desired level. Thus, in some embodiments, the medium is buffered to a pH value of about 4.5, 5.0, 5.5, 6.0, 6.5, 7.0 or 7.5. In some embodiments, the medium is buffered to a pH value of about 5.0. In some embodiments, the medium is buffered to a pH value of about 7.5. If a buffer is not used, the pH of the medium should be maintained at about 4.5 to 8.5, depending upon the particular endoglycoceramidase and other enzymes used.

Enzyme amounts or concentrations can be expressed in activity units, which is a measure of the initial rate of catalysis. One activity unit catalyzes the formation of 1 μmol of product per minute at a given temperature (typically 37° C.) and pH value (typically 7.5). Thus, 10 units of an enzyme is a catalytic amount of that enzyme where 10 μmol of substrate are converted to 10 μmol of product in one minute at a temperature of 37° C. and a pH value of 7.5.

Each of the enzymes is present in a catalytic amount. The catalytic amount of a particular enzyme varies according to the concentration of that enzyme's substrate as well as to reaction conditions such as temperature, time and pH value. Means for determining the catalytic amount for a given enzyme under preselected substrate concentrations and reaction conditions are well known to those of skill in the art.

In carrying out the reactions of the invention, the enzyme is included at a concentration sufficient to produce at least a 50% yield of glycolipid. In some embodiments, at least about 0.01 mg/ml EGCase of the invention is included in a reaction mixture. In some embodiments, at least about 0.02 mg/ml, 0.05 mg/ml, 0.10 mg/ml, 0.20 mg/ml, 0.25 mg/ml, 0.30 mg/ml, 0.50 mg/ml, 0.75 mg/ml, 1.0 mg/ml or greater concentrations of enzyme are included in the reaction mixture. The concentration selected is independent of reaction volume or length of reaction time. For example, reactions can be successfully carried out in larger volumes (e.g., at least about 100 ml) with smaller concentrations of enzyme. Reactions can be carried out for shorter periods of time (e.g., less than about 24 hours) with smaller concentrations of enzyme.

With respect to the concentration relationships of enzyme to acceptor substrate, in some embodiments, at least about 0.1 mg enzyme per mmol of acceptor substrate is included in a reaction mixture. In some embodiments, about 0.2 mg, 0.5 mg, or 0.8 mg enzyme per mmol of acceptor substrate is included in a reaction mixture. In some embodiments, about 1.0 mg, 1.5 mg, 2.0 mg, 5.0 mg, 10 mg, 15 mg, 20 mg, 25 mg, 30 mg, 40 mg, 50 mg, 75 mg, 100 mg or more enzyme per mmol of acceptor substrate is included in a reaction mixture.

With respect to the concentration relationships of enzyme to donor substrate, in some embodiments, at least about 0.1 mg enzyme per mmol of donor substrate is included in a reaction mixture. In some embodiments, about 0.2 mg, 0.5 mg, or 0.8 mg enzyme per mmol of donor substrate is included in a reaction mixture. In some embodiments, about 1.0 mg, 1.5 mg, 2.0 mg, 5.0 mg, 10 mg, 15 mg, 20 mg, 25 mg, 30 mg, 40 mg, 50 mg, 75 mg, 100 mg or more enzyme per mmol of donor substrate is included in a reaction mixture.

The temperature at which an above process is carried out can range from just above freezing to the temperature at which the most sensitive enzyme denatures. That temperature range is preferably about 0° C. to about 45° C., and more preferably at about 20° C. to about 37° C.

The reaction mixture so formed is maintained for a period of time sufficient to obtain the desired high yield of desired glycolipid determinants. For large-scale preparations, the reaction will often be allowed to proceed for between about 0.5-240 hours, for example, about 1-18 hours, about 12-144 hours, for example, about 12, 18, 24, 36, 48, 60, 120 or 144 hours. In some embodiments, the reaction is allowed to proceed for about 3, 4, 5, or 6 days.

Preferably, the concentrations of activating donor substrates and enzymes are selected such that glycosylation proceeds until the acceptor substrate is consumed. In some embodiments, the glycosyl donor and the acceptor substrate (i.e., aglycone) are present in a molar ratio range of from about 2:1 to about 1:2, for example, about 2:1, 1.5:1, 1.4:1, 1.1:1, 1:1, 1:1.1, 1:1.4, 1:1.5, or 1:2. In some embodiments, at least about 5 mM donor substrate is included in a reaction mixture. In some embodiments, from about 5 mM to about 50 mM donor substrate is included in a reaction mixture, for example, about 5 mM, 10 mM, 15 mM, 20 mM, 25 mM, 30 mM, 40 mM, 50 mM, or more as desired, for example, 75 mM or 100 mM. In some embodiments, at least about 5 mM acceptor substrate is included in a reaction mixture. In some embodiments, from about 5 mM to about 50 mM acceptor substrate is included in a reaction mixture, for example, about 5 mM, 10 mM, 15 mM, 20 mM, 25 mM, 30 mM, 40 mM, 50 mM, or more as desired, for example, 75 mM or 100 mM.

The volume of the reaction mixtures will be determined according to the purpose of carrying out the reaction and the amount of glycolipid product desired. Small scale reactions can be carried out in volumes that are less than about 2 milliliters, e.g., about 50-2000 μL, about 50-500 μL, for example, about 50 μL, 100 μL, 200 μL, 300 μL, 500 μL, 1 mL, 1.5 mL or 2 mL. Large-scale reactions can be carried out in volumes greater than about 2 mL, for example, about 5-4000 mL, 10-50 mL, 100-1000 mL, 1-4 liters, for example, about 5 mL, 10 mL, 20 mL, 30 mL, 50 mL, 100 mL, 300 mL, 500 mL, 750 mL, 1 liter, 2 liters, 3 liters, 4 liters, or more. In some embodiments, the large-scale reactions are carried out in volumes of more than 1 liter, for example, 2 L, 3 L, 4 L, 5 L, 10 L, 20 L, 50 L, 100 L or more. Large-scale reactions also can be carried out in volumes of more than 100 L, for example, 200 L, 500 L, 1000 L, 2000 L, 5000 L or more.

The reaction mixtures of the present invention can produce glycolipid product from donor and acceptor substrates in at least about 40% yield, more preferably at least about 45%, 50%, 55%, 60%, 65%, 70%, 75%, 80% yield or more. In large-scale reaction mixtures, at least 100 mg glycolipid product is produced. In some embodiments of large-scale reaction mixtures, at least about 200 mg, 300 mg, 500 mg, or more glycolipid product is produced. In some embodiments, glycolipid is produced on a gram scale, for example, at least about 1 gram, 2 grams, 3 grams, 5 grams, 8 grams, 10 grams, 20 grams, 30 grams, 40 grams, 50 grams, 75 grams, 100 grams, 500 grams or more glycolipid product is produced. In some embodiments, glycolipid is produced on a kilogram scale, for example, 1 kg, 2 kg, 3 kg, 5 kg, 8 kg, 10 kg, 20 kg, 30 kg, 40 kg, 50 kg, 75 kg, 100 kg or more glycolipid product is produced.

### Donor Substrates

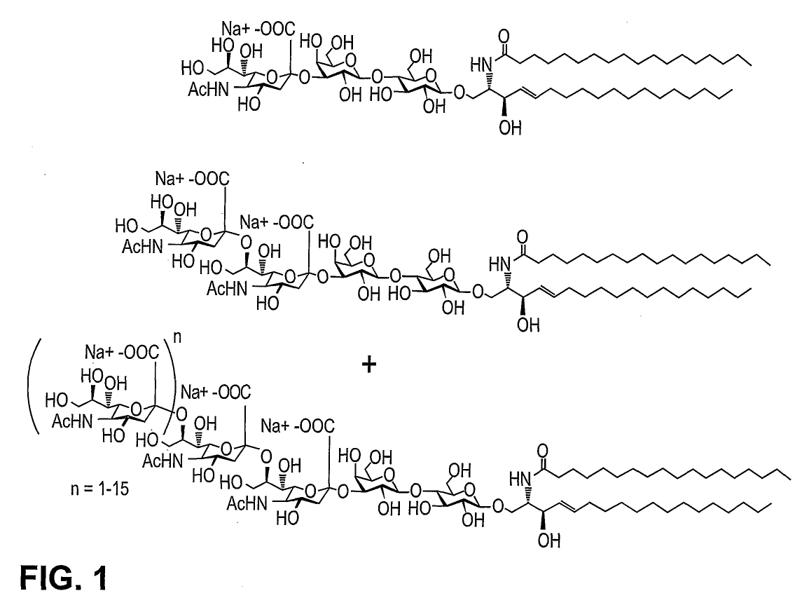

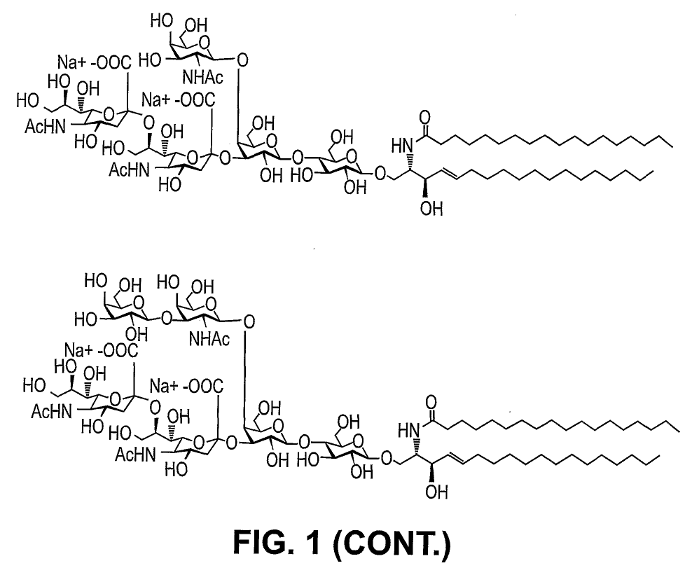

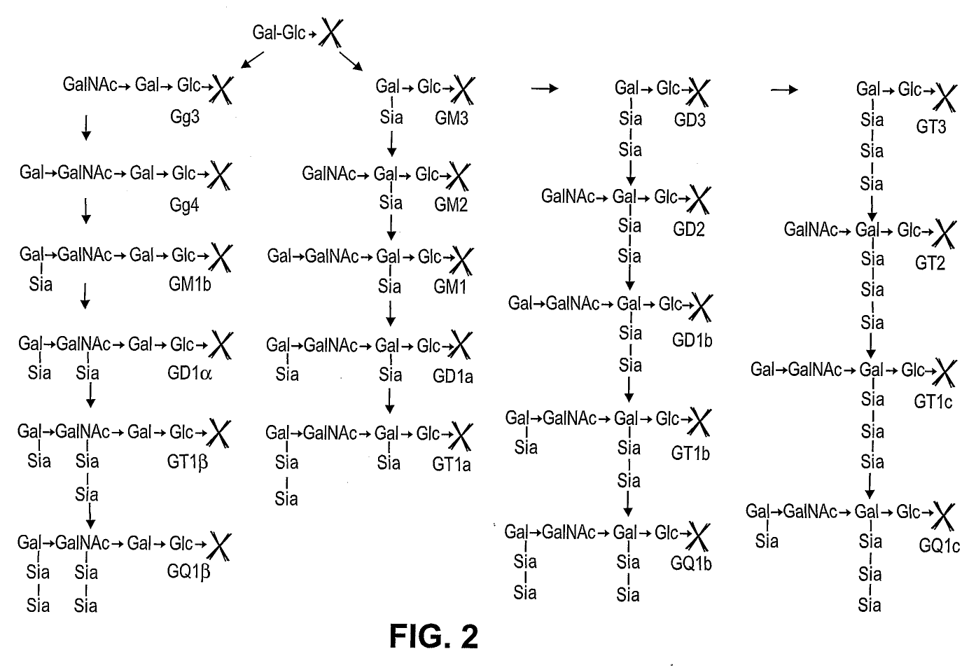

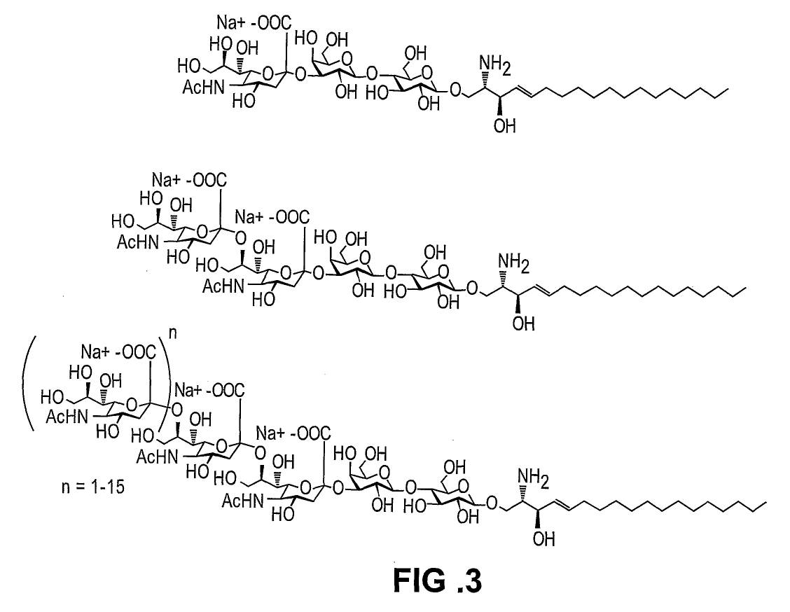

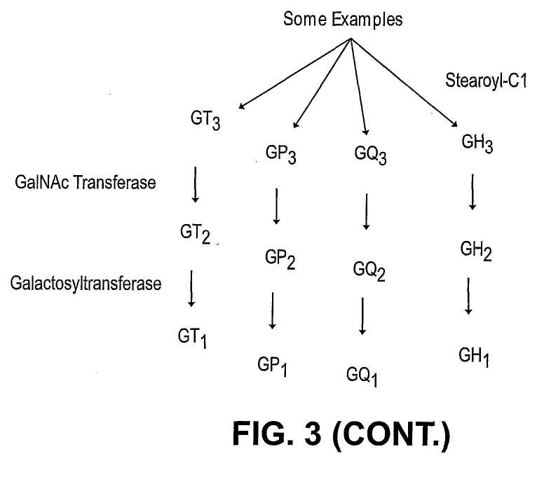

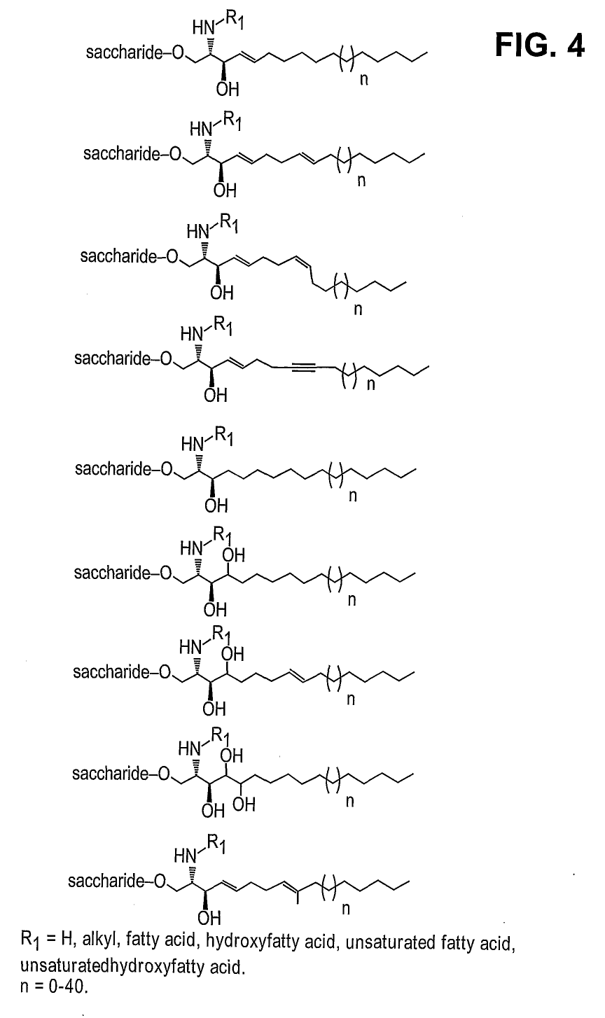

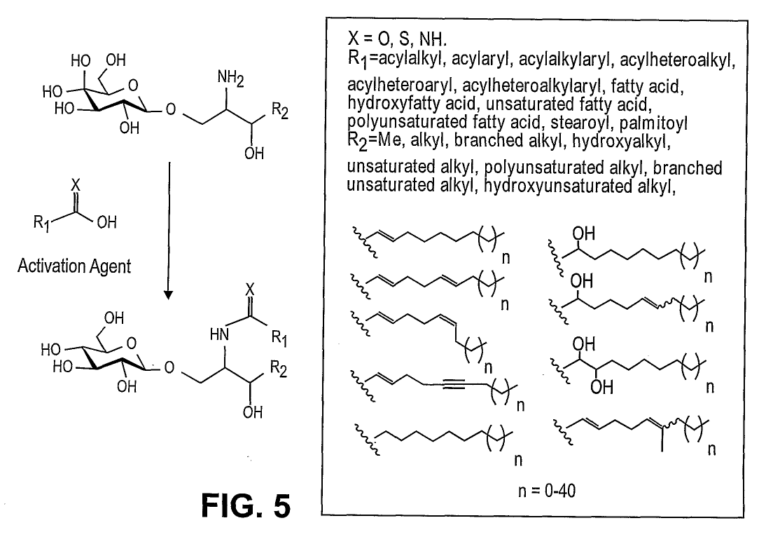

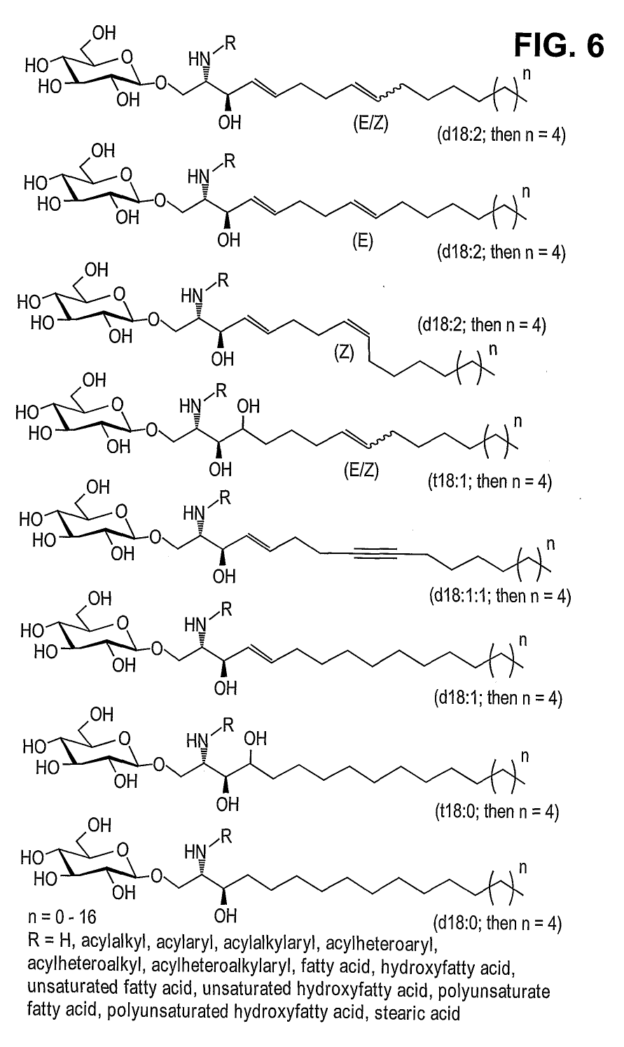

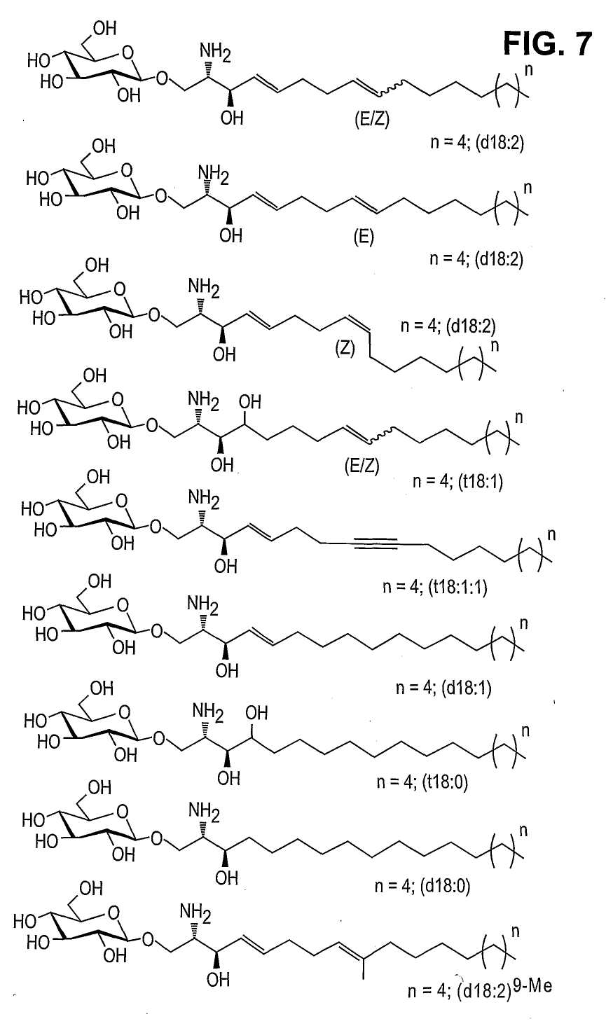

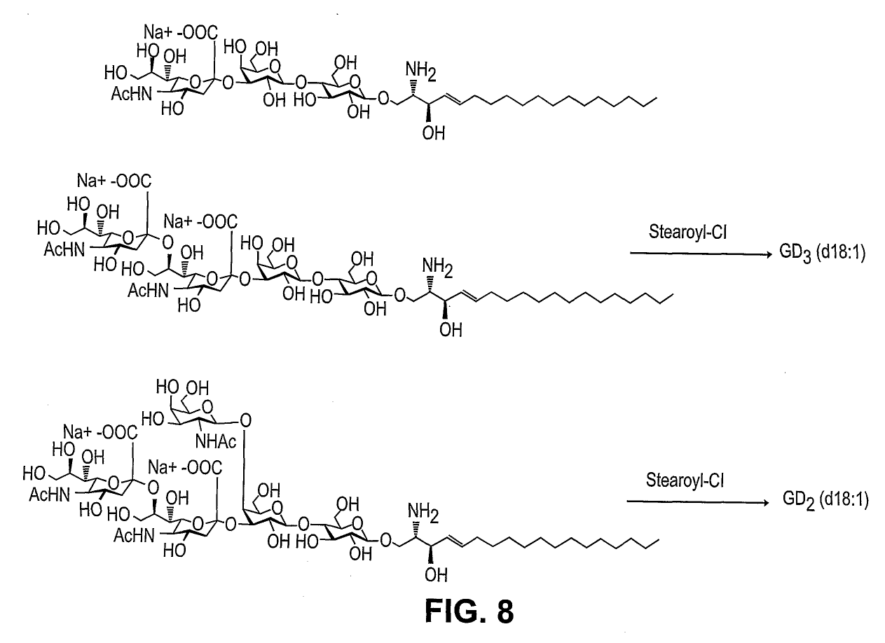

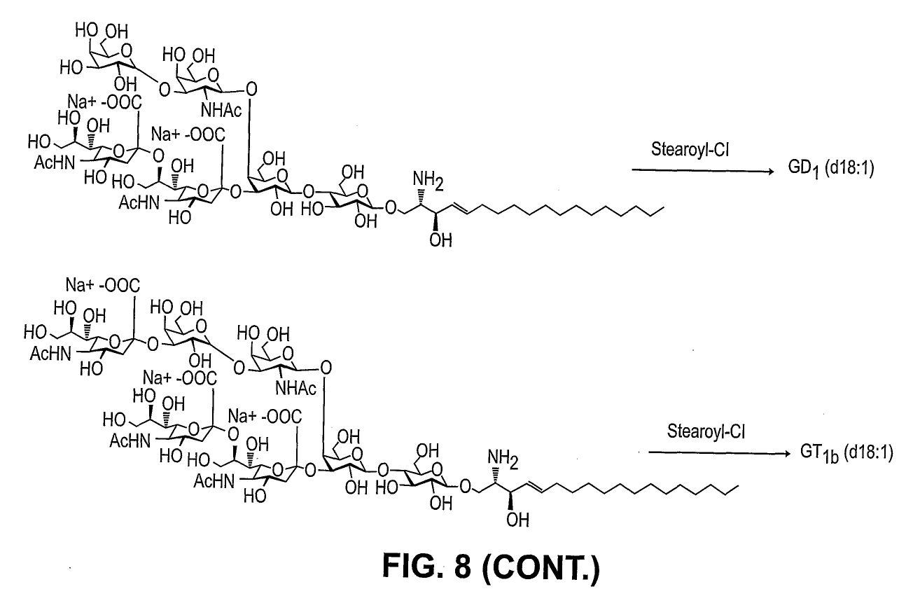

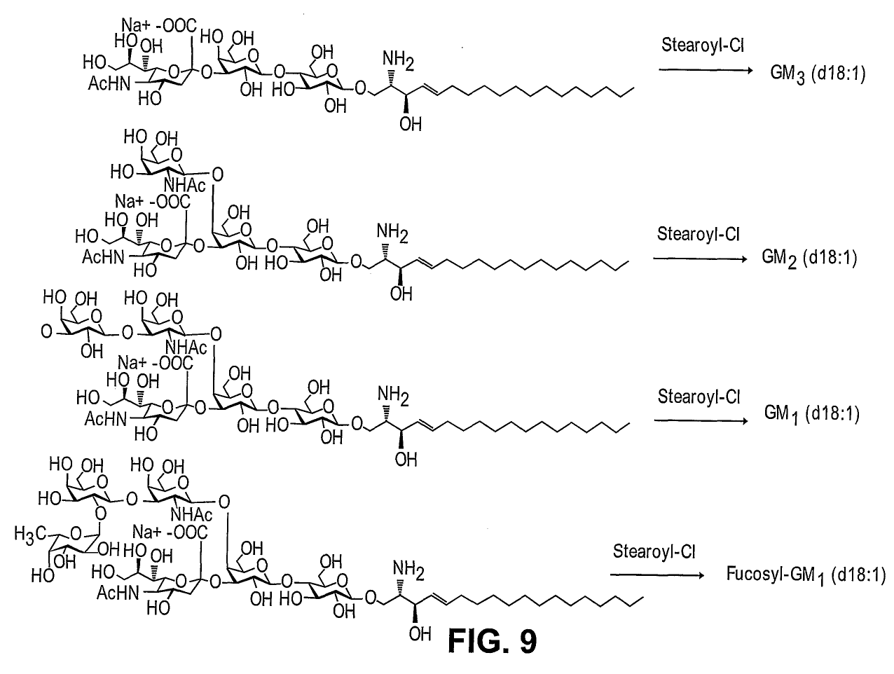

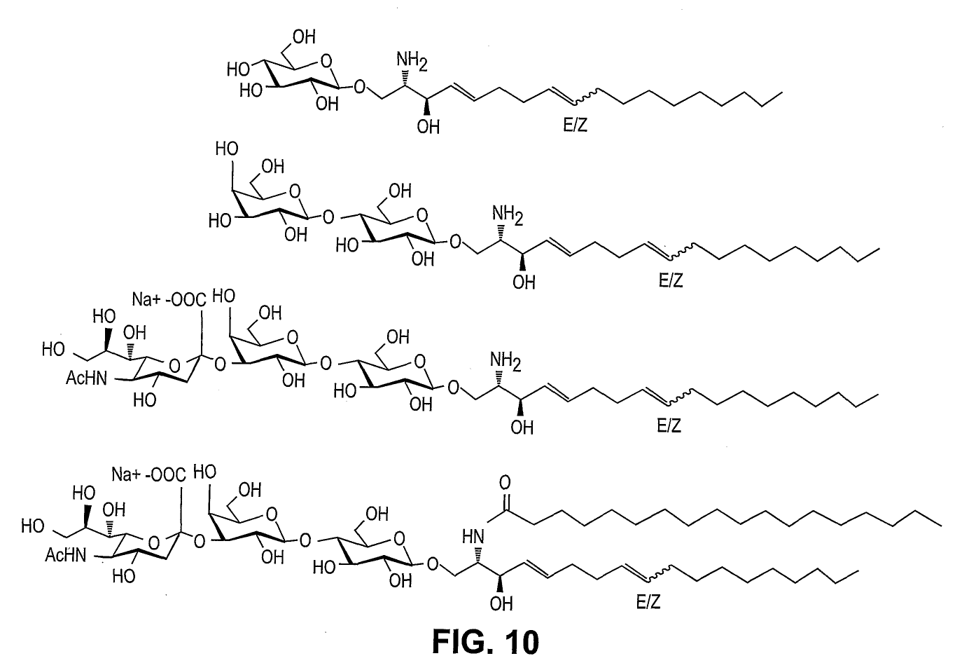

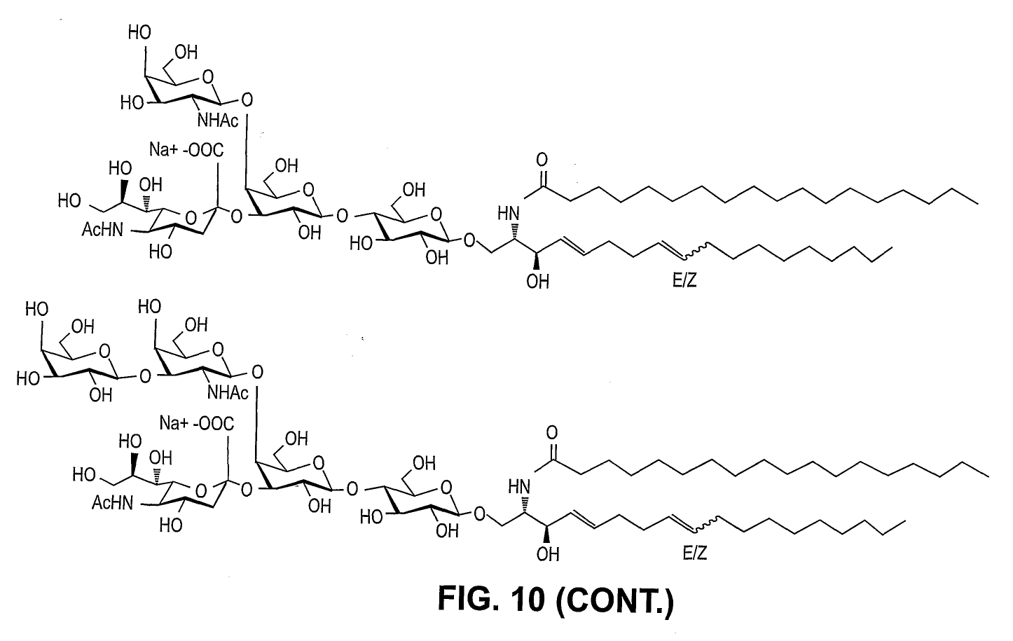

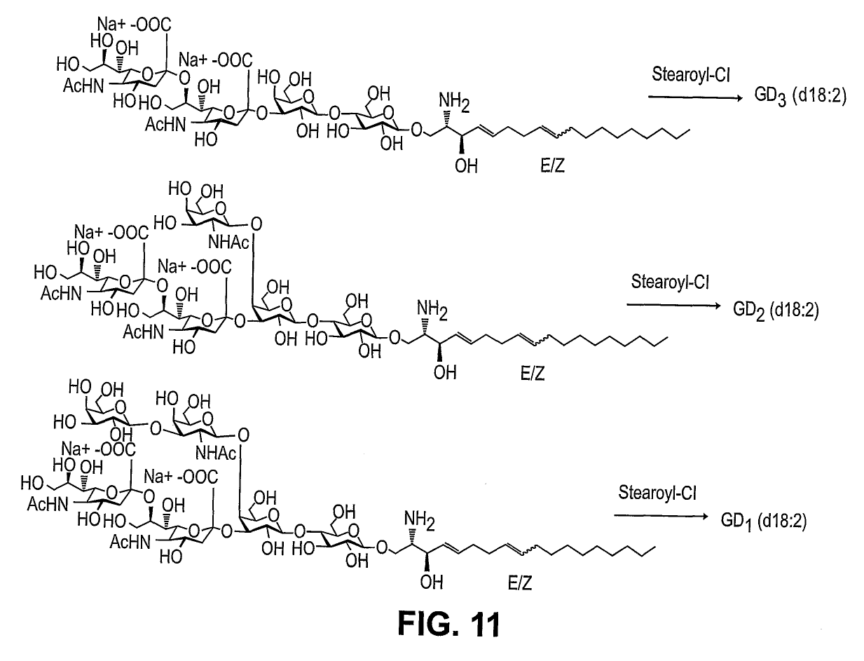

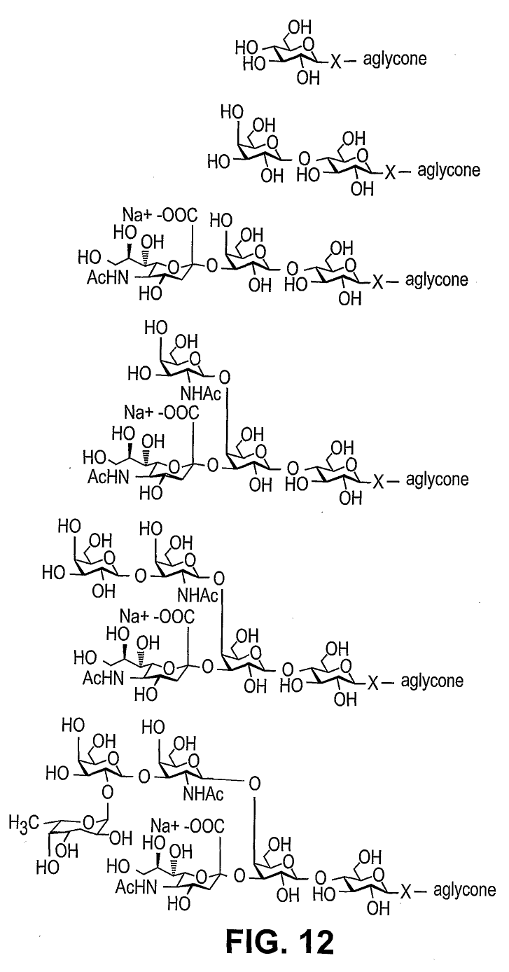

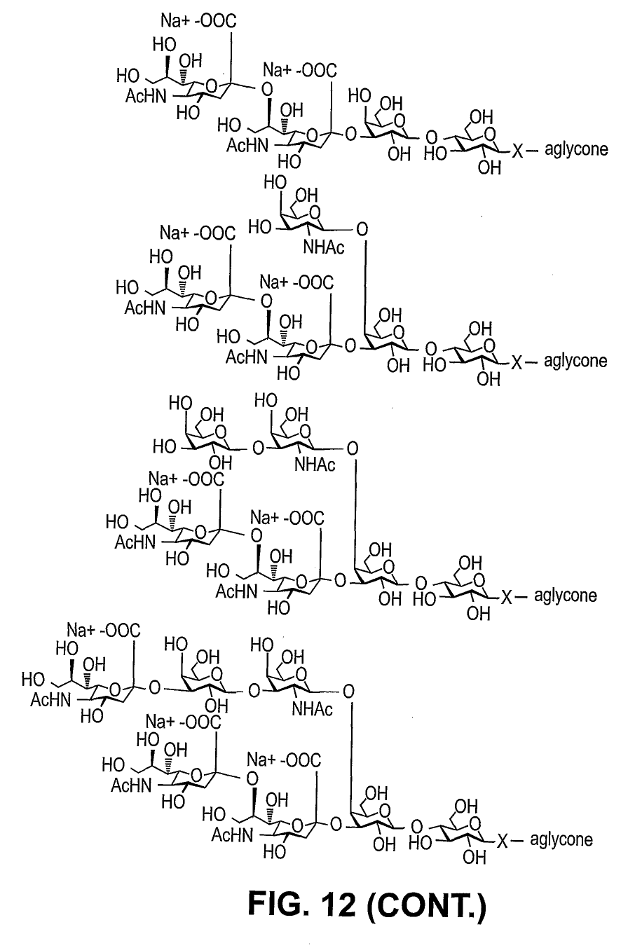

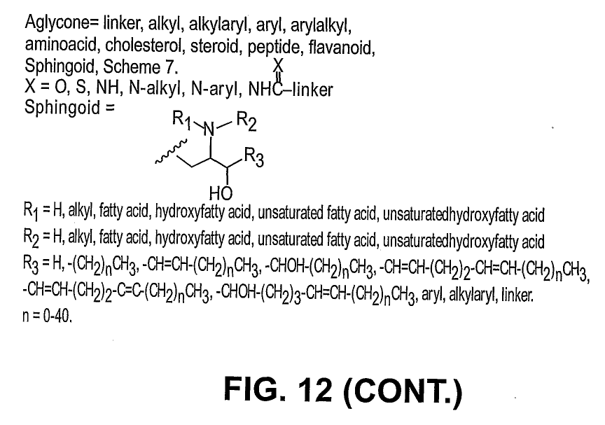

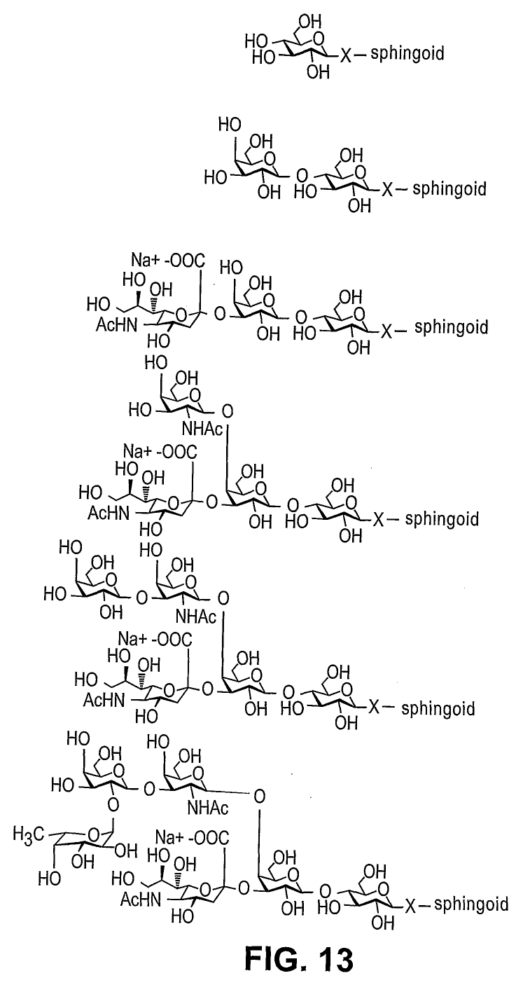

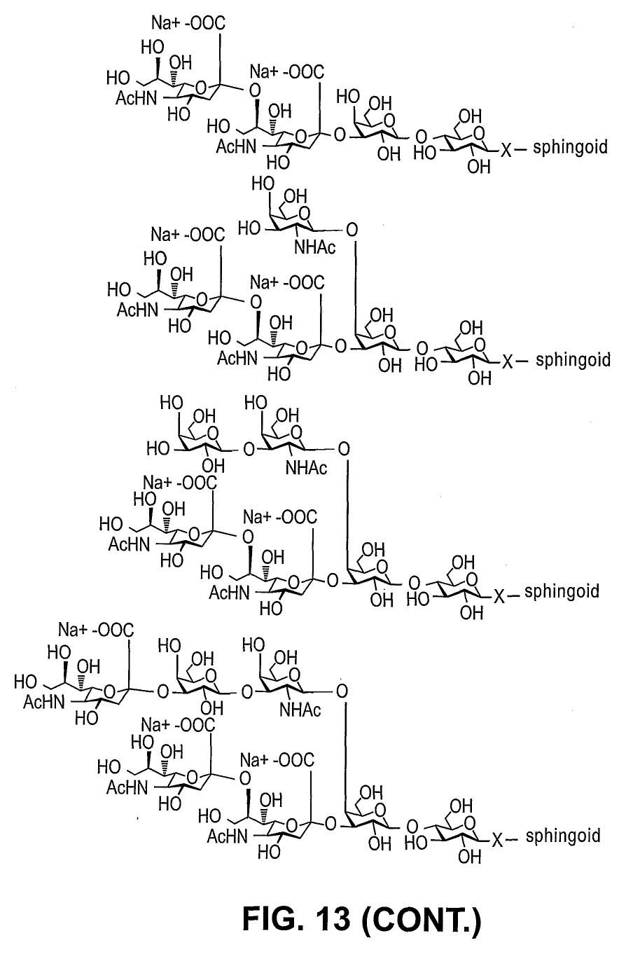

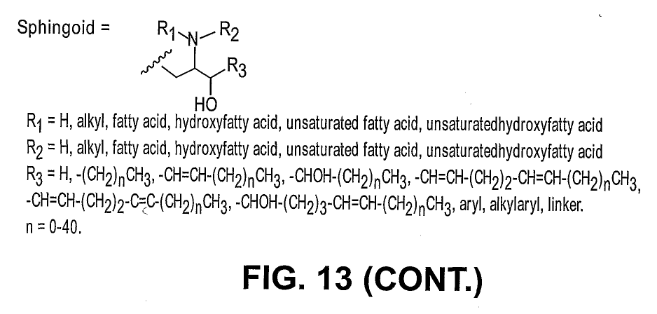

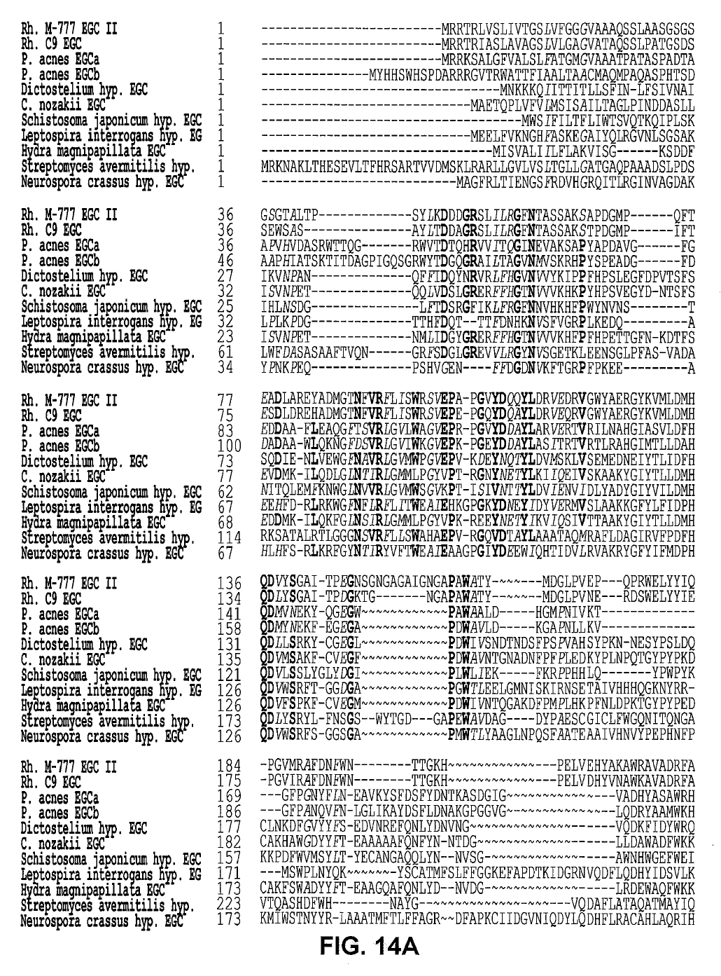

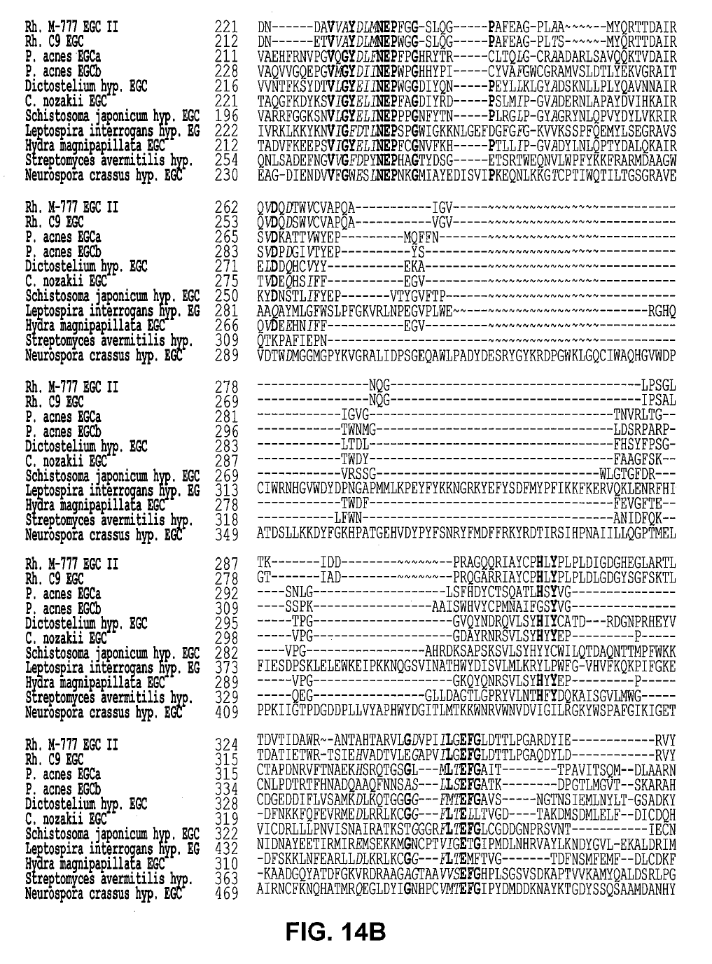

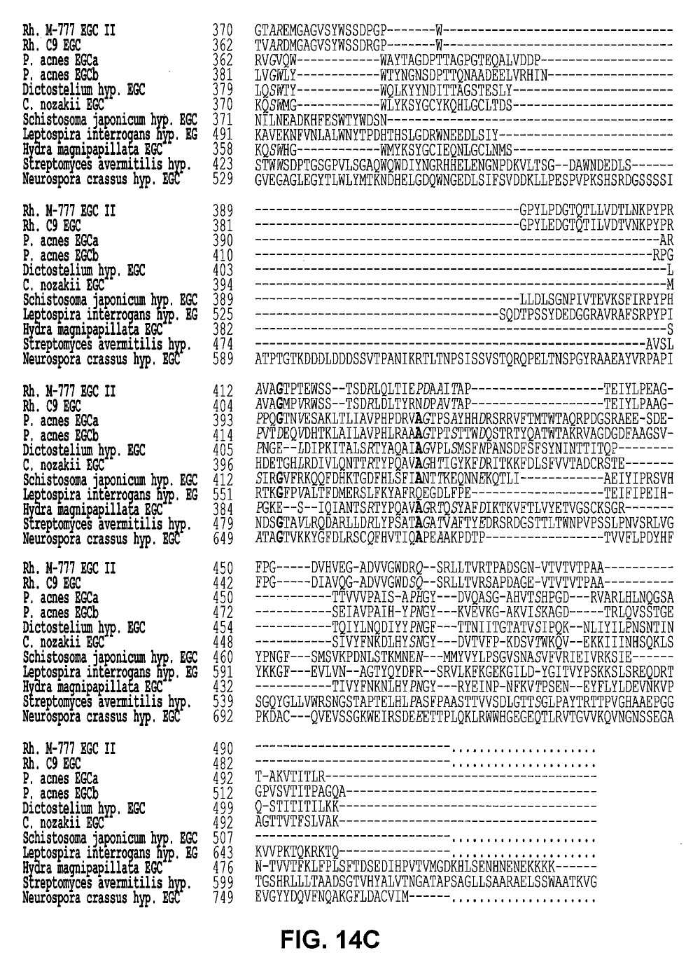

Donor substrates for wild-type and mutant endoglycoceramidases include any activated glycosyl derivatives of anomeric configuration opposite the natural glycosidic linkage. The enzymes of the invention are used to couple α-modified or α-modified glycosyl donors, usually α-modified glycosyl donors, with glycoside acceptors. Preferred donor molecules are glycosyl fluorides, although donors with other groups which are reasonably small and which function as relatively good leaving groups can also be used. Examples of other glycosyl donor molecules include glycosyl chlorides, bromides, acetates, mesylates, propionates, pivaloates, and glycosyl molecules modified with substituted phenols. Among the α-modified or β-modified glycosyl donors, α-galactosyl, α-mannosyl, α-glucosyl, a-fucosyl, α-xylosyl, α-sialyl, α-N-acetylglucosaminyl, α-N-acetylgalactosaminyl, β-galactosyl, β-mannosyl, β-glucosyl, β-fucosyl, β-xylosyl, β-sialyl, β-N-acetylglucosaminyl and β-N-acetylgalactosaminyl are most preferred. Additional donor substrates include ganglioside head groups, for example, those listed in Table 2, below, and those depicted in FIGS. 1-13. Accordingly, in one embodiment, the donor substrate can be one or more ganglioside glycosyl head groups selected from the group consisting Of GD₁ₐ. GD_{1α}, GD_{1b}, GD₂, GD₃, Gg3, Gg4, GH₁, GH₂, GH₃, GM₁, GM_{1b}, GM₂, GM₃, Fuc-GM₁, GP₁, GP₂, GP₃, GQ_{1b}, GQ_{1B}, GQ_{1β}, GQ_{1c}, GQ₂, GQ₃, GT₁ₐ, GT_{1b}, GT_{1c}, GT_{1β}, GT_{1c}, GT₂, and GT₃. The donor molecules can be monosaccharides, or may themselves contain multiple sugar moieties (oligosaccharides). Donor substrates of use in the particular methods include those described in U.S. Pat. Nos. 6,284,494; 6,204,029; 5,952,203; and 5,716,812.

Glycosyl fluorides can be prepared from the free sugar by first acetylating the sugar and then treating it with HF/pyridine. This will generate the thermodynamically most stable anomer of the protected (acetylated) glycosyl fluoride. If the less stable anomer is desired, it may be prepared by converting the peracetylated sugar with HBr/HOAc or with HCL to generate the anomeric bromide or chloride. This intermediate is reacted with a fluoride salt such as silver fluoride to generate the glycosyl fluoride. Acetylated glycosyl fluorides may be deprotected by reaction with mild (catalytic) base in methanol (e.g., NaOMe/MeOH). In addition, glycosyl donor molecules, including many glycosyl fluorides can be purchased commercially. Thus a wide range of donor molecules are available for use in the methods of the present invention.

### Acceptor Substrates

Suitable acceptor substrates include any aglycone that the mutant endoceramidases can conjugate with a saccharide moiety. For example, the mutant endoglycoceramide synthases are capable of synthesizing a glycolipid or aglycone by coupling a saccharide and a heteroalkyl substrate with a structure as shown in Formula Ia, Formula Ib, Formula II or Formula III as shown below:

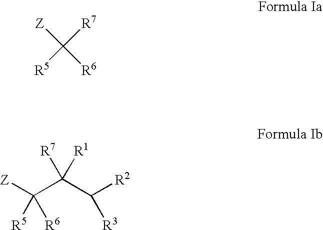

In Formula Ia and Formula Ib, the symbol Z represents OH, SH, or NR⁴R^{4′}. R¹ and R² are members independently selected from NHR⁴, SR⁴, OR⁴, OCOR⁴, OC(O)NHR⁴, NHC(O)OR⁴, OS(O)₂OR⁴, C(O)R⁴, NHC(O)R⁴, detectable labels, and targeting moieties. The symbols R³, R⁴ and R^{4′}, R⁵, R⁶ and R⁷ each are members independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl.

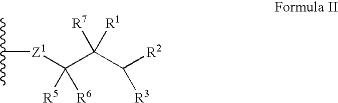

In Formula II, Z¹ is a member selected from O, S, and NR⁴; R¹ and R² are members independently selected from NHR⁴, SR⁴, OR⁴, OCOR⁴, OC(O)NHR⁴, NHC(O)OR⁴, OS(O)₂OR⁴, C(O)R⁴, NHC(O)R⁴, detectable labels, and targeting moieties. The symbols R³, R⁴, R⁵, R⁶ and R⁷ each are members independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl. Formula II is representative of certain embodiments wherein the aglycone portion is conjugated to a further substrate component, for example, a leaving group or a solid support.

In certain embodiments, acceptor substrates such as those depicted in Table 1 below are used in the methods of glycolipid or aglycone synthesis employing the mutant endoglycoceramidases.

TABLE 1
Representative Acceptor Substrates For Glycosynthase Synthesis Reactions

| | A |
|---|---|
| 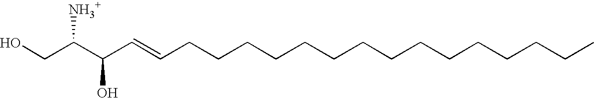 | |
| Acceptor structure | |
| | B |
| 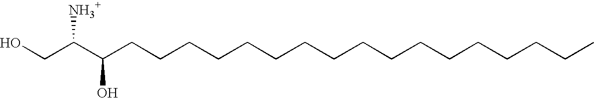 | |
| | C |
| 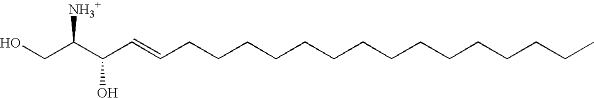 | |
| | D |
| 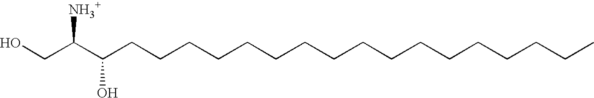 | |
| | E |
| 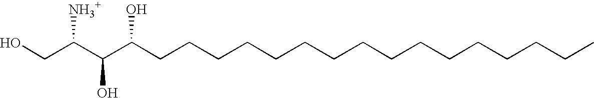 | |
| | F |
| 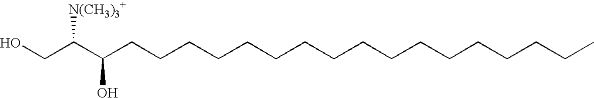 | |
| | G |
| 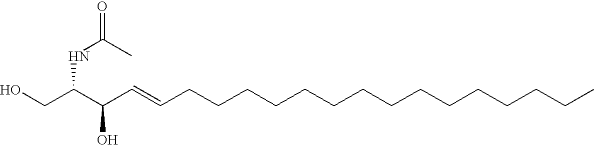 | |
| | H |
| 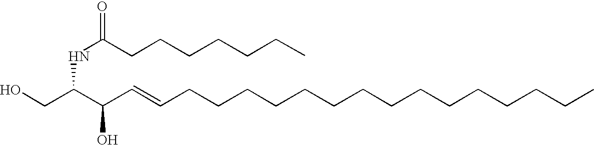 | |
| | I |
| 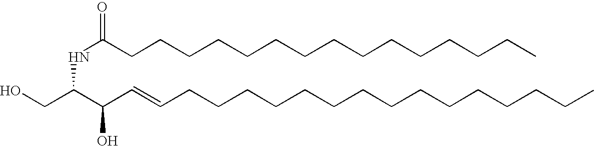 | |
| | J |
| 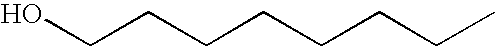 | |

In certain embodiments, the acceptor substrate is a sphingosine, a sphingosine analog or a ceramide. In certain embodiments, the acceptor substrate is one or more sphingosine analogs, including those described in co-pending patent applications PCT/US2004/006904 (which claims priority to U.S. Provisional Patent Application No. 60/452,796); U.S. patent application Ser. No. 10/487,841; U.S. patent application Ser. No. 10/485,892; 10/485,195, and PCT/US2005/040195 (which claims priority to U.S. Provisional Patent Application No. 60/626,678).

In general, the sphingosine analogs described in the above-referenced applications are those compounds having the formula:

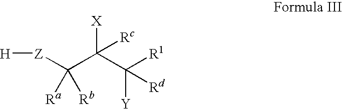

wherein Z is a member selected from O, S, C(R²)₂ and NR²; X is a member selected from H, —OR³, —NR³R⁴, —SR³, and —CHR³R⁴; R¹, R², R³ and R⁴ are members independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl, —C(=M)R⁵, —C(=M)-Z¹-R⁵, —SO₂R⁵, and —SO₃; wherein M and Z¹ are members independently selected from O, NR⁶ or S; Y is a member selected from H, —OR⁷, —SR⁷, —NR⁷R⁸, substituted or unsubstituted alkyl, substituted or unsubstituted heteroalkyl, substituted or unsubstituted aryl, substituted or unsubstituted heteroaryl, and substituted or unsubstituted heterocycloalkyl, wherein R⁵, R⁶, R⁷ and R⁸ are members independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl; and R^{a}, R^{b}, R^{c} and R^{d} are each independently H, substituted or unsubstituted alkyl, substituted or unsubstituted heteroalkyl, substituted or unsubstituted aryl, substituted or unsubstituted heteroaryl, substituted or unsubstituted heterocycloalkyl.

In certain embodiments, the acceptor substrate can be one or more sphingosine analogs including D-eryth/ro-sphingosine, D-erythro-sphinganine, L-threo-sphingosine, L-threo-dihydrosphingosine, D-erythro-phytosphingosine, or N-ocatanoyl-D-erythro-sphingosine.

### Mutant Endoglycoceramidases

The present invention uses mutant endoglycoceramidases, also termed “endoglycoceramide synthases,” which have an increased synthetic activity for attaching a donor substrate comprising a saccharide moiety to an acceptor substrate (an aglycone) compared to the corresponding wild-type endoglycoceramidase. These enzymes have been described in International Application No. PCT/US05/19451 and U.S. Provisional Application Nos. 60/666,765; 60/626,791, 60/576,316, the disclosures of each of which are hereby incorporated herein by reference in their entirety for all purposes. The mutant endoglycoceramidases can also have a reduced hydrolytic activity towards glycolipids compared to the corresponding wild-type endoglycoceramidase. Corresponding wild-type endoglycoceramidases have at least two identifiable conserved motifs, including an acid-base region (Val-X₁-(Ala/Gly)-(Tyr/Phe)-(Asp/Glu)-(Leu/Ile)-X₂-Asn-Glu-Pro-X₃-X₄-Gly or motif B or SEQ ID NO:2), and a nucleophilic region ((Ile/Met/Leu/Phe/Val)-(Leu/Met/Ile/Val)-(Gly/Ser/Thr)-Glu-(Phe/Thr/Met/Leu)-(Gly/Leu/Phe) or motif D or SEQ ID NO:4), and hydrolyze the glycoside linkage between a sugar chain and a lipid moiety in a glycolipid.

Structurally, a mutant endoglycoceramidase useful in the reaction mixtures and methods of the invention usually have a modified nucleophilic carboxylate Glu/Asp residue, wherein the nucleophilic Glu/Asp resides within a (Ile/Met/Leu/Phe/Val)-(Leu/Met/Ile/Val)-(Gly/Ser/Thr)-(Glu/Asp)-(Phe/Thr/Met/Leu)-(Gly/Leu/Phe) sequence (SEQ ID NO:5) of a corresponding wild-type endoglycoceramidase, wherein the mutant endoglycoceramidase catalyzes the transfer of a saccharide moiety from a donor substrate to an acceptor substrate.

A mutant endoglycoceramidase useful in the reaction mixtures and methods of the invention can have a modified Glu residue within the subsequence of Asn-Glu-Pro, wherein the subsequence resides within the acid-base sequence region of Val-X₁-(Ala/Gly)-(Tyr/Phe)-(Asp/Glu)-(Leu/Ile)-X₂-Asn-Glu-Pro-X₃-X₄-Gly sequence in the corresponding wild-type protein, wherein the mutant endoglycoceramidase catalyzes the transfer of a saccharide moiety from a donor substrate to an acceptor substrate.

A mutant endoglycoceramidase useful in the reaction mixtures and methods of the invention can also be characterized in that
i) in its native form the endoglycoceramidase comprises an amino acid sequence that is any one of SEQ ID NOs: 7 (Rhodococcus), 9 (Rhodococcus), 11 (Propionibacterium acnes), 13 (Propionibacterium acnes), 15 (Cyanea nozakii), 17 (Cyanea nozakii), 19 (Hydra magnipapillata), 21 (Schistosoma japonicum), 22 (Dictyostelium discoideum), 23 (Streptomyces avermitilis), 24 (Leptospira interrogans), and 25 (Neurospora crassa); and
ii) the nucleophilic Glu/Asp residue within a (Ile/Met/Leu/Phe/Val)-(Leu/Met/Ile/Val)-(Gly/Ser/Thr)-Glu/Asp-(Phe/Thr/Met/Leu)-(Gly/Leu/Phe) sequence of a corresponding wild-type endoglycoceramidase is modified to an amino acid other than Glu/Asp; or
ii) the Glu residue within the subsequence of Asn-Glu-Pro of the acid-base sequence region Val-X₁-(Ala/Gly)-(Tyr/Phe)-(Asp/Glu)-(Leu/Ile)-X₂-Asn-Glu-Pro-X₃-X₄-Gly in the corresponding wild-type protein is modified to an amino acid other than Glu.

Typically, the mutant endoglycoceramidases comprise a modified nucleophilic Glu/Asp residue and/or a modified acid-base sequence region Glu residue within the Asn-Glu-Pro subsequence of a corresponding wild-type endoglyoceramidase. One or both of the Glu residues are deleted or replaced with another chemical moiety that retains the integral structure of the protein such that the mutant enzyme has synthetic activity. For example, one or more of the nucleophilic and/or acid-base sequence region Glu residues (i.e., in the Asn-Glu-Pro subsequence region) can be replaced with an L-amino acid residue other than Glu, an unnatural amino acid, an amino acid analog, an amino acid mimetic, and the like. Usually, the one or more Glu residues are substituted with another L-amino acid other than Glu, for example, Gly, Ala, Ser, Asp, Asn, Gln, Cys, Thr, Ile, Leu or Val.

Functionally, the mutant endoglycoceramidases have a synthetic activity of coupling a glycosyl moiety and an aglycone substrate, forming a glycolipid. The mutant endoglycoceramidase can also have a reduced hydrolytic activity towards glycolipids compared to the corresponding wild-type endoglycoceramidase. The mutant endoglycoceramidases of the invention have a synthetic activity that is greater than the synthetic activity of the corresponding wild type endoglycoceramidase. Preferably, the synthetic activity is greater than its degradative (i.e., hydrolytic) activity in an assay. The assay for the synthetic activity of the mutant endoglycoceramidase comprises transferring a glycosyl moiety from a glycosyl donor substrate for said mutant to an aglycone (i.e., acceptor substrate). The synthetic activity can be readily measured in an assay designed to detect the rate of glycolipid synthesis by the mutant or the quantity of product synthesized by the enzyme.

In general, preferred mutant endoglycoceramidases are at least about 1.5-fold more synthetically active than their wild type analogues, more preferably, at least about 2-fold, at least about 5-fold, at least about 10-fold, at least about 20-fold, a least about 50-fold and more preferably still, at least about 100-fold. By more synthetically active is meant that the rate of starting material conversion by the enzyme is greater than that of the corresponding wild type enzyme and/or the amount of product produced within a selected time is greater than that produced by the corresponding wild type enzyme in a similar amount of time. A useful assay for determining enzyme synthetic activity includes transferring a glycosyl moiety from a glycosyl donor substrate for said mutant to an aglycone.

The corresponding wild-type endoglycoceramidase can be from a prokaryotic organism (e.g., a Rhodococcus, a Propionibacterium, a Streptomyces, or a Leptospira) or a eukaryotic organism (e.g., a Cyanea, a Hydra, a Schistosoma, a Dictyostelium, a Neurospora). For example, the corresponding wild-type or native endoglycoceramidase can be from an Actinobacteria, including a Rhodococcus, a Propionibacterium, or a Streptomyces. The corresponding wild-type or native endoglycoceramidase also can be from a Metazoan, including a Cyanea, a Hydra, or a Schistosoma, or from a Cnidaria, including a Cyanea or a Hydra. The corresponding wild-type or native endoglycoceramidase also can be from a Mycetozoa (e.g., a Dictyostelium), a Spirochete (e.g., a Leptospira), or a fungus, such as an Ascomycete (e.g., a Neurospora). In one embodiment, the corresponding wild-type endoglycoceramidase has an amino acid sequence of any one of SEQ ID NOs: 7, 9, 11, 13, 15, 17, 19, 21, 22, 23, 24 or 25. In one embodiment, the corresponding wild-type endoglycoceramidase is encoded by a nucleic acid sequence of any one of SEQ ID NOs: 6, 8, 10, 12, 14, 16, 18 or 20.

The corresponding wild-type endoglycoceramidase can be from any known endoglycoceramidase sequence or any endoglycoceramidase sequence which has yet to be determined. Additional corresponding wild-type endoglycoceramidases can be identified using sequence databases and sequence alignment algorithms, for example, the publicly available GenBank database and the BLAST alignment algorithm, available on the worldwide web through ncbi.nlm.nih.gov. Additional corresponding wild-type endoglycoceramidases also can be found using routine techniques of hybridization and recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1987-2005). Native or wild-type endoglycoceramidases of interest include those encoded by nucleic acid sequences that hybridize under stringent hybridization conditions to one or more of SEQ ID NOs: 6, 8, 10, 12, 14, 16, 18 or 20. Native or wild-type endoglycoceramidases of interest also include those with one or more conservatively substituted amino acids or with at least about 80%, 85%, 90%, 95%, 96%, 97%, 98% or 99% sequence identity to one or more of SEQ ID NOs: 7, 9, 11, 13, 15, 17, 19, 21, 22, 23, 24 or 25.

Wild-type and mutant endoglycoceramidases can be further characterized by a (Met/Val/Leu)-Leu-Asp-(Met/Phe/Ala)-His-Gln-Asp-(Met/Val/Leu)-X-(Ser/Asn) motif (motif A or SEQ ID NO:1) located N-terminal to the acid-base sequence region and a C-terminal Ala-Ile-Arg-(Gln/Ser/Thr)-Val-Asp motif (motif C or SEQ ID NO:3) located C-terminal to the acid-base sequence region. For example, the (Met/Val/Leu)-Leu-Asp-(Met/Phe/Ala)-His-Gln-Asp-(Met/Val/Leu) motif is located at residues 131-140 in Rhodococcus sp. M-777; at residues 129-138 in Rhodococcus sp. C9; at residues 136-145 in Propionibacterium acnes EGCa; at residues 153-162 in Propionibacterium acnes EGCb; at residues 130-139 in Cyanea nozakii; and at residues 121-130 in Hydra magnipapillata. The Ala-Ile-Arg-(Gln/Ser/Thr)-Val-Asp motif is located at residues 259-264 in Rhodococcus sp. M-777; at residues 250-255 in Rhodococcus sp. C9; at residues 262-267 in Propionibacterium acnes EGCa; at residues 280-285 in Propionibacterium acnes EGCb; at residues 272-277 in Cyanea nozakii; and at residues 263-268 in Hydra magnipapillata.

Wild-type and mutant endoglycoceramidases can be expressed in prokaryotic and eukaryotic host cells using methods known in the art. See, for example, Cloning, Gene Expression and Protein Purification: Experimental Procedures and Process Rationale, Hardin and Harbin, eds., 2001, Oxford Univ Pr; Recombinant Gene Expression: Reviews and Protocols, Balabas and Lorence, eds., 2004, Humana Pr; and Carson and Robertson, Manipulation And Expression of Recombinant DNA: A Lab Manual, 2005, Academic Pr. Expression of wild-type and mutant endoglycoceramidases is described, for example, in PCT/US05/19451, hereby incorporated herein by reference in its entirety.

To enhance expression of a mutant endoglycoceramidase in the soluble fraction of a bacterial host cell, the mutant endoglycoceramidases typically have had removed the native N-terminal signal peptide sequence that is expressed in the corresponding wild-type enzyme. The signal peptide sequence is typically found within the N-terminal 15, 20, 25, 30, 35, 40, 40, 45, 50 or 55 amino acid residues of a corresponding wild-type endoglycoceramidase.

### Synthesis of Glycolpids Using Mutant Endoglycoceramide Synthases

The methods of the invention can be used for production of a large variety of glycolipids based on different combinations of heteroalkyl substrates. End products of particular interest are glycosylated aglycones, including glycosylated sphingosines, glycosylated sphingosine analogs, and glycosylated ceramides (i.e., cerebrosides and gangliosides). The methods of the invention are useful for producing any of a large number of gangliosides and related structures. Many gangliosides of interest are described in Oettgen, H. F., ed., Gangliosides and Cancer, VCH, Germany, 1989, pp. 10-15, and references cited therein. The end product can be a glycosylsphingosine, a glycosphingolipid, a cerebroside or a ganglioside. Exemplified ganglioside end products include those listed in Table 2, below. Accordingly, in one embodiment, the synthesized glycolipid can be one or more of GD₁ₐ. GD_{1α}, GD_{1b}, GD₂, GD₃, Gg₃, Gg₄, GH₁, GH₂, GH₃, GM₁, GM_{1b}, GM₂, GM₃, Fuc-GM₁, GP₁, GP₂, GP₃, GQ_{1b}, GQ_{1B}, GQ_{1β}, GQ_{1c}, GQ₂, GQ₃, GT₁ₐ, GT_{1b}, GT_{1c}, GT_{1β}, GT_{1c}, GT₂, GT₃, or polysialylated lactose.

TABLE 2
Exemplified Ganglioside Formulas and Abbreviations

| Structure | Abbreviation |
|---|---|
| Neu5Ac3Gal4GlcCer | GM3 |
| GalNAc4(Neu5Ac3)Gal4GlcCer | GM2 |
| Gal3GalNAc4(Neu5Ac3)Gal4GlcCer | GM1a |
| Neu5Ac3Gal3GalNAc4Gal4GlcCer | GM1b |
| Neu5Ac8Neu5Ac3Gal4GlcCer | GD3 |
| GalNAc4(Neu5Ac8Neu5Ac3)Gal4GlcCer | GD2 |
| Neu5Ac3Gal3GalNAc4(Neu5Ac3)Gal4GlcCer | GD1a |
| Neu5Ac3Gal3(Neu5Ac6)GalNAc4Gal4GlcCer | GD1α |
| Gal3GalNAc4(Neu5Ac8Neu5Ac3)Gal4GlcCer | GD1b |
| Neu5Ac8Neu5Ac3Gal3GalNAc4(Neu5Ac3)Gal4GlcCer | GT1a |
| Neu5Ac3Gal3GalNAc4(Neu5Ac8Neu5Ac3)Gal4GlcCer | GT1b |
| Gal3GalNAc4(Neu5Ac8Neu5Ac8Neu5Ac3)Gal4GlcCer | GT1c |
| Neu5Ac8Neu5Ac3Gal3GalNAc4(Neu5Ac8Neu5c3)Gal4GlcCer | GQ1b |

Nomenclature of Glycolipids, IUPAC-IUB Joint Commission on Biochemical Nomenclature (Recommendations 1997); Pure Appl. Chem. (1997) 69: 2475-2487; Eur. J. Biochem (1998) 257: 293-298) (see, the worldwide web at chem.qmw.ac.uk/iupac/misc/glylp.html).

Exemplified end products further include those depicted in FIGS. 1-13. Additional end product glycolipids that can be produced using the mutant endoglycoceramidases of the present invention include the glycosphingolipids, glycosylsphingosines and ganglioside derivatives disclosed in co-pending patent applications PCT/US2004/006904 (which claims priority to U.S. Provisional Patent Application No. 60/452,796); U.S. patent application Ser. No. 10/487,841; U.S. patent application Ser. No. 10/485,892; 10/485,195, and PCT/US2005/040195 (which claims priority to U.S. Provisional Application No. 60/626,678).

Further modifications can be made to the glycolipids synthesized using the endoglycoceramide synthase of the present invention. Exemplary methods of further elaborating glycolipids produced using the present invention are set forth in WO 03/017949; PCT/US02/24574; US2004063911 (although each is broadly directed to modification of peptides with glycosyl moieties, the methods disclosed therein are equally applicable to the glycolipids and method of producing them set forth herein). Moreover, the glycolipid compositions of the invention can be subjected to glycoconjugation as disclosed in WO 03/031464 and its progeny (although each is broadly directed to modification of peptides with glycosyl moieties, the methods disclosed therein are equally applicable to the glycolipids and method of producing them set forth herein).

### EXAMPLES

The following examples are provided by way of illustration only and not by way of limitation. Those of skill in the art will readily recognize a variety of non-critical parameters that could be changed or modified to yield essentially similar results.

### Example 1

### Generating Mutant Endoglycoceramidases

A synthetic endoglycoceramidase gene was produced by Blue Heron Biotechnology (EGCasel395). Subsequently the gene was subcloned into a pT7-7 expression vector. Mutations at one of the nucleotides encoding Glu233 of endoglycoceramidase derived from Rhodococcus sp. M-777 (GenBank Accession No. AAB67050, SEQ ID NO:7), were introduced into the EGCase gene by a PCR-based method using five primer sets by combining the same 5′ primer with five different 3′ primers:

| | | |
|---|---|---|
| The 5′ primer: | | |
| 5′Copt | AATTCGATTGGATCCCATATGAGCGGAAGCG | (SEQ ID NO:45) |
| The 3′ primers: | | |
| 3′Asp PstI | TCGATTCTGCAGGGAGCCACCAAACGGGTCATTCATCAG | (SEQ ID NO:46) |
| 3′Gln PstI | TCGATTCTGCAGGGAGCCACCAAACGGCTGATTCATCAG | (SEQ ID NO:47) |
| 3′Ala PstI-11-1 | CGGTCCCTGCAGGGAGCCACCAAACGGCGCATTCATCAG | (SEQ ID NO:48) |
| 3′Gly PstI-11-1 | CGGTCCCTGCAGGGAGCCACCAAACGGCCCATTCATCAG | (SEQ ID NO:49) |
| 3′Ser PstI-11-1 | CGGTCCCTGCAGGGAGCCACCAAACGGCGAATTCATCAG | (SEQ ID NO:50) |

The PCR program used for generating mutations was essentially as follows: the template and primers were first incubated at 95° C. for 5 minutes, Vent DNA polymerase (New England Biolabs) was then added, which was followed by 30 cycles of amplification: 94° C. for 1 minute, 55° C. for 1 minute, and 72° C. for 2 minutes.

PCR products were digested with NdeI and PstI, and pT7-7 vector was digested with NdeI, EcoRI, and PstI. Following purification of the digestion products from a 0.8% TAE agarose gel, the PCR products were subcloned into pT7-7 vector via a ligation reaction. Upon completion of the ligation reaction, the ligation product was electroporated into BL21DE3 LacZ⁻ cells, which were prepared from BL21DE3 cells (William Studier, Brookhaven National Laboratories, Upton, N.Y.) by disrupting the LacZ gene with a tetracycline or kanamycin resistance gene (generated at Neose Technologies, Inc.). Colonies were screened for PCR product insert. All EGCase mutants were confirmed by sequencing.

### Example 2

### Hydrolytic Assays

An exemplary hydrolytic reaction had a volume of 50 μL, containing 20 μg of substrate (pre-dried lyso-GM2, GM2, or GM3, generated at Neose Technologies, Inc.), 25 μg of Taurodeoxycholic acid (Sigma, Cat # T-0875), 50 mM sodium acetate (pH 5.2), and 5-10 μL of crude cell lysate containing a wild-type or mutant EGCase. The hydrolytic mixture was incubated at 37° C. for 10 to 120 minutes.

### Example 3

### Synthetic Assays

An exemplary synthetic reaction had a volume of 50 μL, containing 5 mM MgCl₂, 0.5% detergent, 0.3 mM ceramide-C-18 (pre-dried), 20 mM Tris-HCl (pH 7.5), and 0.36 mM 3′ sialyl lactose fluoride (3′ SLF). The detergents used in the reaction were Triton-X100 (0.5%), Taurodeoxycholic acid (25 μg), NP-40 (0.5%), Tween-80 (0.5%), 3-14 Zwittergent (0.5%), and Triton-CF54 (0.5%). The reaction times ranged from 2 to 16 h in various buffers ranging in pH from 5.2 to 8.0.

### Example 4

### TLC Analysis

5 μL of a hydrolytic or synthetic reaction was spotted on a TLC plate. The plate was then dried with a hair dryer set on low. The plate was run in an appropriate solvent system (solvent A: chloroform/methanol at 95:5 v/v, solvent B: 1-butyl alcohol/acetic acid/H₂O at 2:1:1 v/v, solvent C: chloroform/methanol/H₂O/ammonium hydroxide at 60:40:5:3). The plate was then dried and stained with anisaldehye. The TLC plate was subsequently developed by heating on a hot plate set at three.

### Example 5

The following example illustrates the successful generation of a glycosynthase enzyme capable of performing the efficient glycosidic coupling between 3′-sialyllactosyl fluoride and a variety of lipid acceptors by performing selected modifications on the endoglycoceramidase II enzyme from Rhodococcus M-777 (SEQ ID NO:7).

Cloning of exemplified mutant endoglycoceramidase E351S

The DNA sequence of the wild-type EGCase gene from Rhodococcus was used as a template for the design of the construct. Using an overlapping PCR strategy, an amino acid substitution of serine for glutamic acid at amino acid position 351 relative to the wild-type enzyme was engineered into the coding sequence. The final coding sequence was also truncated at amino acid 29 relative to the wild-type enzyme in order to mimic the mature version of the enzyme that is normally generated during secretion. Restriction sites were engineered onto the ends of the coding sequence (Nde1 and Xho1, respectively) in order to ligate to the corresponding sites in frame with the six his tag from the pET28A vector (Novagen/EMD Biosciences, San Diego Calif.). This construct was confirmed to be correct by restriction and sequence analysis and then was used to transform the E. coli strain BL21(DE3) (Novagen) using 50 mcg/ml Kanamycin selection. An individual colony was used to inoculate a culture of Maritone-50 mcg/ml Kanamycin that was incubated for 16 hrs at 37° C. A sample of culture was mixed to achieve 20% glycerol and aliquots were frozen at −80° C. and referred as stock vials.

### Mutant Endoglycoceranidase (EGC) Expression and Purification

Wild-type EGC and the following EGC mutants; E351A, E351D, E351D, E351G, and E351S have been successfully expressed and purified. The expression levels for the EGC variants are quite high, therefore cell cultures of 50 ml were used to produce the enzymes.

Cells from a −80° C. freezer stock were directly inoculated into 50 ml Typ broth and were grown at 37° C. to saturation. The temperature was then lowered to 20° C. and protein production was induced by addition of IPTG to 0.1 mM (due to solubility issues, the E351G mutant was expressed at an IPTG concentration of 0.05 mM to prevent aggregation). After 8-12 hours, the cells are harvested by centrifugation and the pellet was resuspended in 2.5 ml BugBuster protein extraction reagent (Novagen). Cell lysis was allowed to proceed for 20 min, and the cell debris was then removed by centrifugation.

The cell lysate was then applied to a 1 ml Ni-NTA column (Amersham), which was then washed with two column volumes of binding buffer (20 mM sodium phosphate, pH 7.0, containing 0.5 M NaCl). EGC was eluted by the stepwise addition of imidazole to a final concentration of 0.5 M (EGC elutes between 0.2 and 0.3 M imidazole). Fractions containing EGC were identified by SDS-PAGE. The purification gave a protein of >95% purity after a single step.

Fractions containing EGC were pooled and the buffer was changed to 25 mM NaOAc, pH 5.0, containing 0.2% Triton X-100 using an Amicon centrifugal ultrafiltration device (MWCO=10,000 Da). At this time, the protein was concentrated to a final volume of approximately 2 ml.

Protein concentration was then assessed using the Bradford method. The purification generally yielded about 10 mg EGC (180-200 mg per liter of expression culture). The enzyme was stable in this form for at least 3 months.

Enzymatic Synthesis of Lyso-GM1 by Mutant EGC Enzymes with Detergent

Reactions were performed in 25 mM of NaOAc buffer (pH 5.0). A typical reaction mixture contained approximately 16 mM of a fluorinated GM1 sugar donor (GM1-F), 16 mM of an acceptor sphingosine, and 0.6 mg/ml of the appropriate EGC mutant in a total reaction volume of 60 μl containing 0.2% Triton X-100. Under these conditions, the reaction proceeds to >90% completion within 12 hours at 37° C. based on normal phase TLC analysis. Running solvent system was CHCl₃/MeOH/H₂O/NH₃H₂O (4/5/1/0.2), and with detection by anisaldehyde stain. Transfer of the fluorinated GM1 sugar donor was also monitored using a reversed phase HPLC method on a Chromolith RP-8e column with eluants of a mixture of 0.1% trifluoroacetic acid (TFA) in acetonitrile (MeCN) and 0.1% TFA in water, with a detection wavelength at 205 nm.

Enzymatic Synthesis of Lyso-GM₃ by Mutant EGC Enzymes with Detergent

Reactions were performed in 25 mM NaOAc (pH 5.0) containing 0.2% Triton X-100. A typical reaction mixture contained approximately 10 mM 3′-sialyllactosyl fluoride (3′-SLF), 20 mM of the acceptor D-e ythro-sphingosine, and 0.5 mg/ml of the appropriate EGC mutant in a total reaction volume of 100 μl. Under these conditions, the reaction proceeds to >90% completion within 12 hours at 37° C. based on TLC analysis. In addition to D-erythro-sphingosine, Table 1, above, shows the structures of other acceptor species that have been used in glycosynthase reactions with 3′-SLF.

Essentially all of the 3′-SLF was consumed in the enzymatic reaction with D-erythro-sphingosine. Thus this reaction delivered a conservative estimate of a minimum of 90% turnover with respect to 3′-SLF. Running solvent was CHCl₃/MeOH/0.2% CaCl₂ (5:4:1), with detection by orcinol-H₂SO₄ stain. Purification of the lyso-GM₃ product was achieved using a combination of normal phase and reversed phase SepPak cartridges (Waters). The identity of the product as lyso-GM₃ was supported by mass spectrometry and NMR.

### Example 6

### Kinetic Parameters of wild-type Rhodococcus M-777 Endoglycoceramidase

Using 2,4-dinitrophenyl lactoside as a substrate, the Rhodococcus M-777 EGC enzyme has a Km of approximately 2 mM, and a kcat of 90 min-1. The dependence of the activity on detergent concentration was also investigated. It was found that in the absence of detergent, the rate of hydrolysis was very low. With the addition of Triton X-100 to 0.1%, the kcat/Km increased dramatically, and gradually decreased with further additions of detergent. The dependence of kcat/Km on detergent concentration leveled off at concentrations greater than 0.5%; increasing the detergent concentration caused a steady increase in both kcat and Km up to a concentration of 1%. The pH dependence of the hydrolysis activity was also investigated. As expected, the maximal kcat/Km is observed around pH 5.

### Example 7

### Expression of Wild-Type Propionibacterium acnes Endoglycoceramidase in E. coli

The expression level of P. acnes EGC enzyme was extremely high, likely exceeding 200 mg/l. However, the expressed protein exclusively formed inclusion bodies under a variety of conditions. This propensity to form inclusion bodies is also observed for the Rhodococcus enzyme, but it is possible to minimize this tendency using Tuner cells in conjunction with a low induction temperature (<20° C.) and low concentration of IPTG (0.1 mM). These tactics proved unsuccessful with the P. acnes enzyme. Furthermore, the P. acnes enzyme was found to express at a very high level even in the absence of IPTG, with inclusion bodies forming during the pre-induction growth phase.

A series of experiments was performed to try to bring at least some protein into the soluble fraction, including:

variation of induction temperature (16-37° C.) in conjunction with variation of [IPTG] (0-0.1 mM);

pre-induction growth at room temperature to lower the levels of background expression;

transformation into BL21 pLysS (to suppress background expression) with variation of conditions as described above;

expression from a lac promoter rather than the T7 system with the above variations;

heat shock of the cells prior to induction (42° C. and 60° C. for 2 min in separate experiments) to induce chaperone expression;

adding a pelB signal sequence to direct secretion into the periplasm; and

attempts were also made to resolubilize the inclusion by denaturation with either urea (8 M) or guanidinium HCL (2 M) as the chaotropic agent followed by either iterative lowering of the denaturant concentration by dialysis or removal of the denaturant by first adsorbing the protein onto a Ni-NTA column and then decreasing the denaturant concentration using a linear gradient.

Soluble P. acnes EGC was obtained by performing the growth and induction steps in M9 minimal medium using Tuner cells with induction overnight at 18° C. in the presence of 0.1 mM IPTG (essentially the same conditions used for the Rhodococcus, except with minimal media rather than rich). In a simultaneous experiment using BL21 pLysS as the expression strain, inclusion bodies were formed, presumably due to the action of the lactose permease in increasing the internal IPTG concentration to a level where expression still proceeds at a very high rate even in minimal media. Simultaneously employing the following three tactics lowered the rate of protein production sufficiently to obtain soluble P. acnes EGC enzyme while retaining the Histag: (i) minimal media for growth and expression, (ii) a very low IPTG concentration, and (iii) expression in the lactose permease deficient Tuner cells. Under these conditions, hydrolysis activity on both 2,4-dinitrophenyl lactoside and GM3 ganglioside in the cell extract was detected.

A gene construct for an E319S mutant EGC was prepared in parallel with the wild-type sequence. This mutant enzyme catalyzed the glycosynthase reaction as well.

### Example 8

### Synthesis of Lyso-GM1 by Mutant EGC Enzymes Using 1,2-dimethoxy Ethane as Co-Solvent

Reactions were performed in 25 mM of NaOAc buffer (pH 5.0) without Triton X-100. A typical reaction mixture contained approximately 16 mM of a fluorinated GM1 sugar donor (GM1-F), 16 mM of an acceptor sphingosine, and 0.6 mg/ml of the appropriate EGC mutant in a total reaction volume of 60 μl. The percentage of the 1,2-dimethyoxy ethane (DME) is 17%. Under these conditions, the reaction proceeds to >90% completion within 12 hours at 37° C. based on normal phase TLC analysis. Running solvent system was CHCl₃/MeOH/H₂O/NH₃H₂O (4/5/1/0.2), and with detection by anisaldehyde stain. Transfer of the fluorinated GM1 sugar donor was also monitored using a reversed phase HPLC method on a Chromolith RP-8e column with eluants of a mixture of 0.1% trifluoroacetic acid (TFA) in acetonitrile (MeCN) and 0.1% TFA in water, with a detection wavelength at 205 nm.

A stock solution of sphingosine (15 mg/ml) with 0.2% Triton-X was also prepared.

Decyl-β-D-maltopyranoside (DM, 2.9 mg) was dissolved in reaction buffer (290 μl) to a final concentration at 10 mg/ml. Doeyl-β-D-maltopyranoside (DDM, 1.7 mg) was dissolved in reaction buffer (170 μl) to a final concentration at 10 mg/ml. Glycerol (3.0 mg) was dissolved in reaction buffer (100 μl) to a final concentration of 30 mg/ml. An endoglycoceramidase, His₆-EGCase E351S, (1.4 mg/ml) was prepared in an aqueous buffer solution of 25 mM NaOAc (pH 5.0) and 300 mM NaCl.

A set of small scale reactions was prepared and incubated at 41° C. Thin layer chromatography (TLC) was checked after 6 hours and after 20 hours. The reactions were also analyzed by high performance liquid chromatography (HPLC). The sample for HPLC was prepared by dissolving 5 μl of reaction mixture in 100 μl of methanol. The results of the reactions are summarized in Table 3, below:

TABLE 3

| | G_{M1}OS-F | Sphingosine | EGCase | | % co- | % | lysoG_{M1}/ |
|---|---|---|---|---|---|---|---|
| | (50 mg/ml) | (15 mg/ml) | (1.4 mg/ml) | Others | solvent | Triton-X | sphingosine |
| 1 | 20 μl | 20 μl | 20 μl | — | — | 0.067% | 8.46 |
| | | (Triton X) | | | | | |
| 2 | 20 μl | 20 μl | 20 μl | Glycerol | — | 0.062% | 8.57 |
| | | (Triton X) | | (50 μl, 0.15 mg) | | | |
| 3 | 20 μl | 20 μl | 20 μl | — | 16.7% | — | 14.3 |
| | | (DME) | | | | | |
| 4 | 20 μl | 20 μl | 20 μl | Glycerol | 15.4% | — | 12.2 |
| | | (DME) | | (5 μl, 0.15 mg) | | | |
| 5 | 20 μl | 20 μl | 20 μl | DDM | 14.3% | — | 9.15 |
| | | (DME) | | (10 μl, 0.1 mg) | | | |
| 6 | 20 μl | 20 μl | 20 μl | DM | 14.3% | — | 13.1 |
| | | (DME) | | (10 μl, 0.1 mg) | | | |

### Example 9

### Enzymatic Synthesis of Lyso-GM1 by Mutant EGC Enzymes Using Glycerol as Co-Solvent

Reactions were performed in 25 mM of NaOAc buffer (pH 5.0). A typical reaction mixture contained approximately 16 mM of a fluorinated GM1 sugar donor (GM1-F), 16 mM of an acceptor sphingosine, and 0.6 mg/ml of the appropriate EGC mutant in a total reaction volume of 60 μl. The percentage of the glycerol is 14%. Under these conditions, the reaction proceeds to >90% completion within 12 hours at 37° C. based on normal phase TLC analysis. Running solvent system was CHCl₃/MeOH/H₂O/NH₃H₂O (4/5/1/0.2), and with detection by anisaldehyde stain. Transfer of the fluorinated GM1 sugar donor was also monitored using a reversed phase HPLC method on a Chromolith RP-8e column with eluants of a mixture of 0.1% trifluoroacetic acid (TFA) in acetonitrile (MeCN) and 0.1% TFA in water, with a detection wavelength at 205 nm.

A set of small scale reactions were prepared and incubated at 41° C. for 2 days. The reactions were analyzed by TLC and HPLC. The samples for HPLC were prepared by dissolving 5 μl of reaction mixture in 100 μl of methanol. The results are summarized in Table 4, below.

TABLE 4

| | G_{M1}OS-F | Sphingosine | EGCase | | % co- | % | lysoG_{M1}/ |
|---|---|---|---|---|---|---|---|
| | (50 mg/ml) | (15 mg/ml) | (1.4 mg/ml) | Others | solvent | Triton-X | sphingosine |
| 1 | 20 μl | 20 μl | 20 μl | — | — | 0.067% | 12.1 |
| | | (Triton X) | | | | | |
| 2 | 20 μl | 20 μl | 20 μl | Glycerol | 14.3% | 0.057% | 12.4 |
| | | (Triton X) | | (10 μl) | | | |
| 3 | 20 μl | 20 μl | 20 μl | — | 16.7% | — | 18.1 |
| | | (DME) | | | | | |

### Example 10

### Large Scale Synthesis of Lyso-GM1 by Mutant EGC Enzyme Using 1,2-dimethoxy Ethane and Glycerol as Co-Solvents

Two solutions of sphingosine HCl salt (18.0 g) and fluorinated GM1 sugar donor (GM1-F, 37.2 g) were each prepared in 25 mM NaOAc reaction buffer at pH 5.0 (1400 mL) with 1,2-dimethyoxy ethane (DME, 300 mL), and his6-EGCase E351S (500 mL, 1.0 mg/mL in 50% glycerol) in 3 L polypropylene flasks. The resulting solutions (total volume 2300 mL), contained 15.5 mM GM1-F, 21.3 mM sphingosine, 13% DME, 11% glycerol and h is 6-EGCase E351S at 0.22 mg/ml. This light brown reaction mixtures were incubated at 37° C. for 6 days until no further conversion to lyso-GM1 occurred. The reaction progress was monitored by normal phase TLC analysis (CHCl₃/MeOH/H₂O/NH₃H₂O, 4/5/1/0.2), with detection by anisaldehyde stain and also by reversed phase HPLC (Chromolith RP-8e column with eluants of a mixture of 0.1% trifluoroacetic acid (TFA) in acetonitrile (MeCN) and 0.1% TFA in water), with a detection wavelength at 205 nm. Then the mixtures were each treated with 1.0 N NaOH (150 mL) to adjust the pH to 12.5, and maintained at 37° C. in the incubator for 18 hrs. HPLC and TLC analysis confirmed the complete hydrolysis. The mixtures were then treated with acetic acid (50 mL) to bring the pH down to 4.3.

The two parallel reaction mixtures were combined and purified on a 75 L reverse phase Metaflash C18 column by step elution of increased percentage of methanol in water from 20% to 90%. Fractions were collected and checked by TLC. Selected fractions were also checked by HPLC analysis. The fractions containing pure lyso-GM1 were combined, concentrated by rotary evaporation, and dried under high vacuum to give 40.1 g (44% yield) of a slightly yellow solid as the desired product.

### Example 11

### Large Scale Synthesis of Lyso-GM1 by Mutant EGC Enzymes Using 1,2-dimethoxy Ethane and Glycerol as Co-Solvents

Two solutions of sphingosine HCl salt (16.0 g) and fluorinated GM1 sugar donor (GM1-F, 34.0 g) were each prepared in 25 mM NaOAc reaction buffer at pH 5.0 (1150 mL), with 1,2-dimethyoxy ethane (DME, 500 ml), and his6-EGCase E351S (700 mL, 1.0 mg/ml in 50% glycerol) in 3 liter polypropylene flasks. The resulting solutions (total volume of the reaction mixture is about 2400 mL) contained 13.6 mM GM1-F, 19.8 mM sphingosine, 10.5% DME, 14.5% glycerol and his6-EGCase E351S at 0.29 mg/mL. This light brown reaction mixtures were incubated at 40° C. for 4 days until no further conversion to lyso-GM1 occurred. The reaction progress was monitored by normal phase TLC (CHCl₃/MeOH/H₂O/NH₃H₂O, 4/5/1/0.2) with detection by anisaldehyde stain. The reactions were also monitored using a reversed phase HPLC (Chromolith RP-8e column with eluants of a mixture of 0.1% trifluoroacetic acid (TFA) in acetonitrile (MeCN) and 0.1% TFA in water), with a detection wavelength at 205 nm. Then the mixtures were treated with 1.0 N NaOH (150 mL) to adjust the pH to 12.3, and maintained at 37° C. in the incubator for 18 hrs. HPLC and TLC analysis confirmed the complete hydrolysis. The mixtures were then treated with acetic acid (50 mL) to bring the pH down to 4.3.

The two parallel reaction mixtures were combined and purified on a 75 L reverse phase Metaflash C18 column by step elution of increased percentage of methanol in water from 20% to 90%. Fractions were collected and checked by TLC. Selected fractions were also checked by HPLC analysis. The fractions containing pure lyso-GM1 were combined, concentrated by rotary evaporation, and dried under high vacuum to give 38.5 g (46% yield) slightly yellow solid as the desired product. About 7 g (8%) of additional impure product was also obtained.

### Example 12

### Large Scale Synthesis of Lyso-GM1 by Mutant EGC Enzyme Using 1,2-dimethoxy Ethane and Glycerol as Co-Solvents

A solution of sphingosine HCl salt (17.7 g) and fluorinated GM1 sugar donor (GM1-F, 50.0 g) was prepared in 25 mM NaOAc reaction buffer at pH 5.0 (2000 mL), with 1,2-dimethyoxy ethane (DME, 450 mL), and his6-EGCase E351S (700 mL, 1.0 mg/ml in 50% glycerol) in a 4 liter polypropylene flask. The resulting mixture (about 3200 mL) contained 15.0 mM GM1-F, 16.5 mM sphingosine, 14% DME, 11% glycerol and his6-EGCase E351S at 0.22 mg/ml. This light brown reaction mixture was incubated at 37° C. for 3 days until no further conversion to lyso-GM1 occurred. The reaction progress was monitored by normal phase TLC (CHCl₃/MeOH/H₂O/NH₃H₂O, 4/5/1/0.2), with detection by anisaldehyde stain. It was also monitored using a reversed phase HPLC (Chromolith RP-8e column with eluants of a mixture of 0.1% trifluoroacetic acid (TFA) in acetonitrile (MeCN) and 0.1% TFA in water) with a detection wavelength at 205 nm. Then the mixture was treated with 12 N NaOH (10 mL) to adjust the pH to 12.0, and maintained at 37° C. in the incubator for 18 hrs. HPLC and TLC analysis confirmed the complete hydrolysis. The mixture was then treated with acetic acid (50 mL) to bring the pH down to 5.

The reaction mixture was purified on a 65 (L or M) reverse phase Metaflash C18 column with step elution of increased percentage of methanol in water from 20% to 90%. Fractions were collected and checked by TLC. Selected fractions were also checked by HPLC analysis. The fractions containing pure lyso-GM1 were combined, concentrated by rotary evaporation, and dried under high vacuum to give 29.8 g (49% yield) slightly yellow solid as the desired product.

### Example 13

### Large Scale Synthesis of Lyso-GM1 by Mutant EGCase Enzyme Using 1,2-dimethoxy Ethane and Glycerol as Co-Solvents

Sphingosine HCl salt (5.04 g) and fluorinated GM1 sugar donor (GM1-F, 10.43 g) were combined in 25 mM of NaOAc reaction buffer at pH 5.0 (600 ml) with 1,2-dimethyoxy ethane (DME, 100 ml), and his6-EGCase E351S (180 mL, 1.0 mg/ml in 50% glycerol) in a 1 liter sterile polycarbonate bottle. The resulting solution (approximately 890 mL) contained 11.2 mM GM1-F, 16.8 mM sphingosine, 15% DME, 10% glycerol and his6-EGCase E351S at 0.02 mg/mL. This light brown reaction mixture was incubated at 37° C. for 3 days until no further conversion to lyso-GM1 occurred. The reaction progress was monitored by normal phase TLC analysis (CHCl₃/MeOH/H₂O/NH₃H₂O: 4/5/1/0.2), with detection by anisaldehyde stain, and also by reversed phase HPLC (Chromolith RP-8e column with eluants of a mixture of 0.1% trifluoroacetic acid (TFA) in acetonitrile (MeCN) and 0.1% TFA in water), with a detection wavelength at 205 nm. The mixture was then treated with 1.0 N NaOH (50 mL) to adjust the pH to 12.0, and maintained at 37° C. in the incubator for 18 hrs. HPLC and TLC analysis confirmed complete acetate hydrolysis. The mixture was then treated with acetic acid (10 mL) to bring the pH down to 5.

The reaction mixture was purified on a 40M reverse phase Metaflash C18 column with step elution of increased percentage of methanol in water from 20% to 90%. Fractions were collected and checked by TLC. The fractions containing pure lyso-GM1 were combined, concentrated by rotary evaporation, and dried under high vacuum to provide 8.14 g (64% yield) of a slightly yellow solid as the desired product.

### Example 14

### Large Scale Synthesis of Lyso-GM1 by Mutant EGC Enzyme Using 1,2-dimethoxy Ethane and Glycerol as Co-Solvents

A solution of sphingosine HCl salt (3.70 g) and fluorinated GMI sugar donor (GM1-F, 10.43 g) was prepared in 25 mM NaOAc reaction buffer at pH 5.0 (400 mL), 1,2-dimethyoxy ethane (DME, 100 mL), and his6-EGCase E351S (160 mL, 1.0 mg/ml in 50% glycerol) in a 1 liter sterile polycarbonate bottle. The reaction mixture (total volume 670 mL) contained 14.9 mM GM1-F, 16.4 mM sphingosine, 15% of DME, 12% of glycerol and his6-EGCase 351S at 0.24 mg/ml. This light brown reaction mixture was incubated at 37° C. for 3 days until no further conversion to lyso-GM1 occurred. The reaction progress was monitored by normal phase TLC (CHCl₃/MeOH/H₂O/NH₃H₂O, 4/5/1/0.2), with detection by anisaldehyde stain. It was also monitored using a reversed phase HPLC (Chromolith RP-8e column with eluants of a mixture of 0.1% trifluoroacetic acid (TFA) in acetonitrile (MeCN) and 0.1% TFA in water) with a detection wavelength at 205 nm. Then the mixture was treated with 1.0 N NaOH (50 mL) to adjust the pH to 12.0, and maintained at 37° C. in the incubator for 18 hrs. HPLC and TLC analysis confirmed the complete hydrolysis. The mixture was then treated with acetic acid (10 mL) to bring the pH down to 5.

The reaction mixture was purified on a 40M reverse phase Metaflash C18 column with step elution of increased percentage of methanol in water from 20% to 90%. Fractions were collected and checked by TLC. The fractions containing pure lyso-GM1 were combined, concentrated by rotary evaporation, and dried under high vacuum to provide 6.38 g (50% yield) slightly yellow solid as the desired product.

### Example 15

### Large Scale Synthesis of Lyso-GM3 by Mutant EGCase Enzyme Using Triton X-100 and Glycerol as Co-Solvents

Sphingosine (300 mg) was dissolved in 10 mL of a 1:1 mixture of methanol and chloroform and Triton X-100 (94 μL) was added. The mixture was evaporated with a stream of nitrogen in a water bath at 40° C. The resulting solid was re-suspended in 25 mM NaOAc reaction buffer at pH 5.0 (25 mL). The fluorinated GM3 sugar donor (GM3-F, 318 mg) and his6-EGCase E351S (25 ml, 1.0 mg/mL in 50% glycerol) were added to the sphingosine solution. The resulting solution (50 mL) contained 20 mM sphingosine, 10 mM GM3-F, 0.2% Triton X-100, 25% glycerol and his6-EGCase E351S mutant at 0.5 mg/mL. This reaction mixture was incubated about 48 hrs at 37° C. The reaction progress was monitored by normal phase TLC analysis (CHCl₃/MeOH/H₂O/NH₃H₂O: 4/5/1/0.2), with detection by anisaldehyde stain. It was also monitored using a reversed phase HPLC method (Chromolith RP-8e column with eluants of a mixture of 0.1% trifluoroacetic acid (TFA) in acetonitrile (MeCN) and 0.1% TFA in water), with detection at 205 nm.

The reaction mixture was purified on a 40S reverse phase Metaflash C18 column with step elution of increased percentage of methanol in water from 20% to 90%. Fractions were collected and checked by TLC. The fractions containing lyso-GM3 were combined, and further purified on the same 40S reverse phase Metaflash C18 column with step elution of increased percentage of acetonitrile in water from 20% to 40%. The combined fractions were concentrated by rotary evaporation, and lyophilized to give 332 mg (73% yield) of white solid as the desired product.

### Example 16

### Large Scale Synthesis of Lyso-GM3 by Mutant EGC Enzyme Using 1,2-dimethoxy Ethane and Glycerol as Co-Solvents

Sphingosine (225 mg) and fluorinated GM3 sugar donor (GM3-F, 318 mg) were combined in 25 mM of NaOAc reaction buffer at pH 5.0 (19 ml) with 1,2-dimethyoxy ethane (DME, 5 ml) and his6-EGCase E351S (8 mL, 1.0 mg/ml in 50% glycerol) in a 50 mL polypropylene centrifuge tube. The resulting solution (32 mL) contained 23.4 mM sphingosine, 15.6 mM GM3-F, 15.6% DME, 12.5% of glycerol, and his6-EGCase E351S mutant at 0.25 mg/ml. This reaction mixture was incubated at 37° C. about 48 hrs. The reaction progress was monitored by normal phase TLC analysis (CHCl₃/MeOH/H₂O/NH₃H₂O: 4/5/1/0.2) with detection by anisaldehyde stain. It was also monitored using a reversed phase HPLC (Chromolith RP-8e column with eluants of a mixture of 0.1% trifluoroacetic acid (TFA) in acetonitrile (MeCN) and 0.1% TFA in water) with detection at 205 nm.

The reaction mixture was purified on a 40S reverse phase Metaflash C18 column with step elution of increased percentage of acetonitrile in water from 20% to 40%. The lyso-GM3 fractions were combined, concentrated by rotary evaporation, and lyophilized to give 325 mg (71% yield) of white solid as the desired product.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A reaction mixture comprising:
a) a solvent comprising at least one of a polyhydric alcohol or an alkoxy ether; and
b) a mutant endoglycoceramidase having a modified nucleophilic carboxylate amino acid residue, wherein the nucleophilic carboxylate residue resides within a (Ile/Met/Leu/Phe/Val)-(Leu/Met/Ile/Val)-(Gly/Ser/Thr)-(Glu/Asp)-(Phe/Thr/Met/Leu)-(Gly/Leu/Phe) sequence (motif E or SEQ ID NO:5).

2. The reaction mixture of claim 1, further comprising a donor substrate comprising a saccharide moiety and an acceptor substrate.

3. The reaction mixture of claim 1, wherein the alkoxy ether is a lower alkoxy ether.

4. The reaction mixture of claim 1, wherein the alkoxy ether is a diether.

5. The reaction mixture of claim 1, wherein the alkoxy ether is a glyme.

6. The reaction mixture of claim 1, wherein the alkoxy ether is 1,2-dimethoxyethane.

7. The reaction mixture of claim 1, wherein the polyhydric alcohol is glycerol.

8. The reaction mixture of claim 1, wherein the wherein the nucleophilic carboxylate amino acid residue is a Glu residing within a (Ile/Met/Leu/Phe/Val)-(Leu/Met/Ile/Val)-(Gly/Ser/Thr)-Glu-(Phe/Thr/Met/Leu)-(Gly/Leu/Phe) sequence (motif D or SEQ ID NO:4).

9. The reaction mixture of claim 1, wherein the corresponding wild-type endoglycoceramidase is selected from the group consisting of a Rhodococcus endoglycoceramidase, a Propionibacterium endoglycoceramidase, a Dictyostelium endoglycoceramidase, a Cyanea endoglycoceramidase, a Hydra endoglycoceramidase, a Streptomyces endoglycoceramidase, a Schistosoma endoglycoceramidase, a Leptospira endoglycoceramidase, and a Neurospora endoglycoceramidase.

10. The reaction mixture of claim 8, wherein the nucleophilic Glu residue is substituted with an amino acid selected from the group consisting of Gly, Ala, Ser, Asp, Asn, Gln, Cys, Thr, Ile, Leu and Val.

11. The reaction mixture of claim 2, wherein the donor substrate is an α-modified glycosyl donor of anomeric configuration opposite the natural glycosidic linkage.

12. The reaction mixture of claim 11, wherein the donor substrate is a glycosyl fluoride.

13. The reaction mixture of claim 2, wherein the acceptor substrate is an aglycone of Formulas Ia, Ib, II or III.

14. The reaction mixture of claim 13, wherein the acceptor substrate is a sphingosine or a sphingosine analog.

15. The reaction mixture of claim 14, wherein the sphingosine is selected from the group consisting of from D-erythro-sphingosine, D-erythro-sphinganine, L-threo-sphingosine, L-threo-dihydrosphingosine, D-erythro-phytosphingosine, N-ocatanoyl-D-erythro-sphingosine.

16. The reaction mixture of claim 13, wherein the acceptor substrate is a ceramide.

17. The reaction mixture of claim 1, wherein the reaction mixture is large-scale.

18. A method of synthesizing a glycolipid, the method comprising, contacting in a reaction mixture comprising at least one of an alkoxy ether and a polyhydric alcohol, a donor substrate comprising a saccharide moiety and an acceptor substrate with a mutant endoglycoceramidase having a modified nucleophilic carboxylate amino acid residue, wherein the nucleophilic carboxylate residue resides within a (Ile/Met/Leu/Phe/Val)-(Leu/Met/Ile/Val)-(Gly/Ser/Thr)-(Glu/Asp)-(Phe/Thr/Met/Leu)-(Gly/Leu/Phe) sequence (motif E or SEQ ID NO:5) of a corresponding wild-type endoglycoceramidase, under conditions wherein the endoglycoceramidase catalyzes the transfer of a saccharide moiety from a donor substrate to an acceptor substrate, thereby producing the glycolipid.

19. The method of claim 18, wherein the alkoxy ether is a lower alkoxy ether.

20. The method of claim 18, wherein the alkoxy ether is a diether.

21. The method of claim 18, wherein the alkoxy ether is a glyme.

22. The method of claim 18, wherein the alkoxy ether is 1,2-dimethoxyethane.

23. The method of claim 18, wherein the polyhydric alcohol is glycerol.

24. The method of claim 18, wherein the wherein the nucleophilic carboxylate amino acid residue is a Glu residing within a (Ile/Met/Leu/Phe/Val)-(Leu/Met/Ile/Val)-(Gly/Ser/Thr)-Glu-(Phe/Thr/Met/Leu)-(Gly/Leu/Phe) sequence (motif D or SEQ ID NO:4).

25. The method of claim 18, wherein the corresponding wild-type endoglycoceramidase is selected from the group consisting of a Rhodococcus endoglycoceramidase, a Propionibacterium endoglycoceramidase, a Dictyostelium endoglycoceramidase, a Cyanea endoglycoceramidase, a Hydra endoglycoceramidase, a Streptomyces endoglycoceramidase, a Schistosoma endoglycoceramidase, a Leptospira endoglycoceramidase, and a Neurospora endoglycoceramidase.

26. The method of claim 24, wherein the nucleophilic Glu residue is substituted with an amino acid selected from the group consisting of Gly, Ala, Ser, Asp, Asn, Gln, Cys, Thr, Ile, Leu and Val.

27. The method of claim 18, wherein the donor substrate is an α-modified glycosyl donor of anomeric configuration opposite the natural glycosidic linkage.

28. The method of claim 27, wherein the donor substrate is a glycosyl fluoride.

29. The method of claim 18, wherein the acceptor substrate is an aglycone of Formulas Ia, Ib, II or III.

30. The method of claim 29, wherein the acceptor substrate is a sphingosine or a sphingosine analog.

31. The method of claim 30, wherein the sphingosine is selected from the group consisting of from D-erythro-sphingosine, D-erythro-sphinganine, L-threo-sphingosine, L-threo-dihydrosphingosine, D-erythro-phytosphingosine, N-octanoyl-D-erythro-sphingosine.

32. The method of claim 29, wherein the acceptor substrate is a ceramide.

33. A large-scale reaction mixture comprising:
a) a solvent comprising at least one of a polyhydric alcohol or an alkoxy ether; and
b) a mutant endoglycoceramidase having a modified nucleophilic carboxylate amino acid residue, wherein the nucleophilic carboxylate residue resides within a (Ile/Met/Leu/Phe/Val)-(Leu/Met/Ile/Val)-(Gly/Ser/Thr)-(Glu/Asp)-(Phe/Thr/Met/Leu)-(Gly/Leu/Phe) sequence (motif E or SEQ ID NO:5);
c) at least 100 mg of a donor substrate comprising a saccharide moiety; and
d) at least 100 mg of an acceptor substrate.

34. The reaction mixture of claim 33, wherein the alkoxy ether is a lower alkoxy ether.

35. The reaction mixture of claim 33, wherein the alkoxy ether is a diether.

36. The reaction mixture of claim 33, wherein the alkoxy ether is a glyme.

37. The reaction mixture of claim 33, wherein the alkoxy ether is 1,2-dimethoxyethane.

38. The reaction mixture of claim 33, wherein the polyhydric alcohol is glycerol.

39. The reaction mixture of claim 33, wherein the wherein the nucleophilic carboxylate amino acid residue is a Glu residing within a (Ile/Met/Leu/Phe/Val)-(Leu/Met/Ile/Val)-(Gly/Ser/Thr)-Glu-(Phe/Thr/Met/Leu)-(Gly/Leu/Phe) sequence (motif D or SEQ ID NO:4).

40. The reaction mixture of claim 33, wherein the corresponding wild-type endoglycoceramidase is selected from the group consisting of a Rhodococcus endoglycoceramidase, a Propionibacterium endoglycoceramidase, a Dictyostelium endoglycoceramidase, a Cyanea endoglycoceramidase, a Hydra endoglycoceramidase, a Streptomyces endoglycoceramidase, a Schistosoma endoglycoceramidase, a Leptospira endoglycoceramidase, and a Neurospora endoglycoceramidase.

41. The reaction mixture of claim 39, wherein the nucleophilic Glu residue is substituted with an amino acid selected from the group consisting of Gly, Ala, Ser, Asp, Asn, Gln, Cys, Thr, Ile, Leu and Val.

42. The reaction mixture of claim 34, wherein the donor substrate is an α-modified glycosyl donor of anomeric configuration opposite the natural glycosidic linkage.

43. The reaction mixture of claim 42, wherein the donor substrate is a glycosyl fluoride.

44. The reaction mixture of claim 34, wherein the acceptor substrate is an aglycone of Formulas Ia, Ib, II or III.

45. The reaction mixture of claim 44, wherein the acceptor substrate is a sphingosine or a sphingosine analog.

46. The reaction mixture of claim 45, wherein the sphingosine is selected from the group consisting of from D-erythro-sphingosine, D-erythro-sphinganine, L-threo-sphingosine, L-threo-dihydrosphingosine, D-erythro-phytosphingosine, N-ocatanoyl-D-erythro-sphingosine.

47. The reaction mixture of claim 44, wherein the acceptor substrate is a ceramide.

48. A large-scale method of synthesizing a glycolipid, the method comprising, contacting in a large-scale reaction mixture comprising at least one of an alkoxy ether and a polyhydric alcohol, a donor substrate comprising a saccharide moiety and an acceptor substrate with a mutant endoglycoceramidase having a modified nucleophilic carboxylate amino acid residue, wherein the nucleophilic carboxylate residue resides within a (Ile/Met/Leu/Phe/Val)-(Leu/Met/Ile/Val)-(Gly/Ser/Thr)-(Glu/Asp)-(Phe/Thr/Met/Leu)-(Gly/Leu/Phe) sequence (motif E or SEQ ID NO:5) of a corresponding wild-type endoglycoceramidase, under conditions wherein the endoglycoceramidase catalyzes the transfer of a saccharide moiety from a donor substrate to an acceptor substrate, thereby producing at least 100 mg of the glycolipid.

49. The method of claim 48, wherein the alkoxy ether is a lower alkoxy ether.

50. The method of claim 48, wherein the alkoxy ether is a diether.

51. The method of claim 48, wherein the alkoxy ether is a glyme.

52. The method of claim 48, wherein the alkoxy ether is 1,2-dimethoxyethane.

53. The method of claim 48, wherein the polyhydric alcohol is glycerol.

54. The method of claim 48, wherein the wherein the nucleophilic carboxylate amino acid residue is a Glu residing within a (Ile/Met/Leu/Phe/Val)-(Leu/Met/Ile/Val)-(Gly/Ser/Thr)-Glu-(Phe/Thr/Met/Leu)-(Gly/Leu/Phe) sequence (motif D or SEQ ID NO:4).

55. The method of claim 48, wherein the corresponding wild-type endoglycoceramidase is selected from the group consisting of a Rhodococcus endoglycoceramidase, a Propionibacterium endoglycoceramidase, a Dictyostelium endoglycoceramidase, a Cyanea endoglycoceramidase, a Hydra endoglycoceramidase, a Streptomyces endoglycoceramidase, a Schistosoma endoglycoceramidase, a Leptospira endoglycoceramidase, and a Neurospora endoglycoceramidase.

56. The method of claim 54, wherein the nucleophilic Glu residue is substituted with an amino acid selected from the group consisting of Gly, Ala, Ser, Asp, Asn, Gln, Cys, Thr, Ile, Leu and Val.

57. The method of claim 48, wherein the donor substrate is an α-modified glycosyl donor of anomeric configuration opposite the natural glycosidic linkage.

58. The method of claim 57, wherein the donor substrate is a glycosyl fluoride.

59. The method of claim 48, wherein the acceptor substrate is an aglycone of Formulas Ia, Ib, II or III.

60. The method of claim 59, wherein the acceptor substrate is a sphingosine or a sphingosine analog.

61. The method of claim 60, wherein the sphingosine is selected from the group consisting of from D-erythro-sphingosine, D-erythro-sphinganine, L-threo-sphingosine, L-threo-dihydrosphingosine, D-erythro-phytosphingosine, N-octanoyl-D-erythro-sphingosine.

62. The method of claim 59, wherein the acceptor substrate is a ceramide.


SEQUENCE LISTING (51 sequences)

SEQ ID NO: 1 - protein, 10 aa, Artificial Sequence
  Description of Artificial Sequencewild-type endoglycoceramidase identifying conserved motif A
XLDXHQDXXX

SEQ ID NO: 2 - protein, 13 aa, Artificial Sequence
  Description of Artificial Sequencewild-type endoglycoceramidase identifying conserved motif B including acid-base sequence region
VXXXXXXNEPXXG

SEQ ID NO: 3 - protein, 6 aa, Artificial Sequence
  Description of Artificial Sequencewild-type endoglycoceramidase identifying conserved C-terminal motif C
AIRXVD

SEQ ID NO: 4 - protein, 6 aa, Artificial Sequence
  Description of Artificial Sequencewild-type endoglycoceramidase identifying nucleophilic region conserved motif D, including nucleophilic Glu residue
XXXEXX

SEQ ID NO: 5 - protein, 6 aa, Artificial Sequence
  Description of Artificial Sequencewild-type endoglycoceramidase identifying nucleophilic region conserved motif E, including nucleophilic carboxylate Glu/Asp residue
XXXXXX

SEQ ID NO: 6 - DNA, 2012 bp, Rhodococcus sp.
  Rhodococcus sp, strain M-177 endoglycoceramidase II, glycosphingolipid-specific endoglycosidase, ceramide glycanase (EGCase)
cctgaccatgttgggccccaacggtttcaggagggagttcgccggggcgaccgacggggccgcggccgaactcgagctgtcctcgacgatcgtcgccgggacgcgatctctcgctctgagcgtgaacaaccgtggaacgcacgagctgacggtcgcggtcgacggtcaacggcgccgggtcgcggcccacgggtcggaatcactgacggtgtcctcggtgaacggttggtacgaggccgccgtgaccgtcgacgaggaccccgacttccggcgacggctcgtcgggcacatcgagaacgggcaggacagcgtcagtcagccgagctgacggggtgtcgccggtaccccggcaaggaacgtgatcgaaccaagagtccagtaggaggacacgtgcgtcgcacccggctcgtatcgctgatcgtgacaggttcgctggtgttcggcggcggcgttgccgccgctcagagcagcttggccgcatccggaagcggaagtggcagtggtaccgcgctgacgccgtcctacctgaaggacgatgacggccgctcactgatcctgcgcgggttcaacacggcatcgagcgcgaagagcgcgccggacggcatgccgcagttcaccgaggcggacctggcgcgcgagtatgcagacatgggaaccaacttcgttcggttcctcatctcgtggcggtcggtcgaaccagcaccgggcgtgtacgaccagcagtatctggaccgtgtcgaagatcgggtcggctggtacgccgagcgcggctacaaggtgatgctcgacatgcaccaggacgtgtactccggcgcgatcaccccggagggcaacagcggcaacggtgccggcgccatcggcaacggcgcaccggcctgggcgacctacatggacggccttccggtcgagccgcagccccggtgggagctgtactacatccagcccggcgtgatgcgcgcgttcgacaacttctggaacaccaccggcaagcaccccgaactcgtcgagcactacgcgaaagcgtggcgggcggtcgccgaccgattcgccgacaacgacgccgtcgtggcctacgacctgatgaacgagccgttcggaggatccctgcagggaccggcgttcgaggcagggccgctcgccgcgatgtaccagcgcaccaccgacgccatccggcaggtagaccaggacacctgggtctgcgtggccccgcaggcgatcggcgtcaaccagggtctccccagcgggctcaccaagatcgacgaccctcgtgcgggtcaacagcgcatcgcgtactgcccgcacctctacccactgccgctggatatcggtgacggccacgagggcctggcccggacgctcaccgacgtgaccatcgacgcctggcgtgccaacaccgcccacaccgcccgtgtgctgggtgacgtgcccatcatcctcggcgagttcggcctggacacaacgctgcccggggcccgggattacatcgaacgcgtctacgggaccgcgcgagagatgggggccggagtctcgtactggtccagcgatcccggcccctggggcccgtacctgcctgacggcacgcagacgctgctcgtcgacaccctgaacaagccgtacccccgcgcagtggccggcacacccaccgagtggtcgtcgacctccgatcgcctccaattgacgatcgagccggacgccgcgatcaccgctcccaccgagatctacctcccggaggcaggattcccgggcgacgtccacgtcgaaggcgccgacgtcgtggggtgggatcggcagagtcgactgctcacggtgcgcactccggccgactcgggcaacgtgaccgtgacggtcactccggcagcctgatccggccgacgcgacgaccggccgtcggtgcgacgatgactgcatggatgaagtggtctcggtctacgacgcagacggcaccgtgatcggcacggcgccacgctcgcgcgtgtacgccgaggggctgtggcatgccagtgcgggcgtgc

SEQ ID NO: 7 - protein, 490 aa, Rhodococcus sp.
  Rhodococcus sp, strain M-177 endoglycoceramidase II, glycosphingolipid-specific endoglycosidase, ceramide glycanase (EGCase)
MRRTRLVSLIVTGSLVFGGGVAAAQSSLAASGSGSGSGTALTPSYLKDDDGRSLILRGFNTASSAKSAPDGMPQFTEADLAREYADMGTNFVRFLISWRSVEPAPGVYDQQYLDRVEDRVGWYAERGYKVMLDMHQDVYSGAITPEGNSGNGAGAIGNGAPAWATYMDGLPVEPQPRWELYYIQPGVMRAFDNFWNTTGKHPELVEHYAKAWRAVADRFADNDAVVAYDLMNEPFGGSLQGPAFEAGPLAAMYQRTTDAIRQVDQDTWVCVAPQAIGVNQGLPSGLTKIDDPRAGQQRIAYCPHLYPLPLDIGDGHEGLARTLTDVTIDAWRANTAHTARVLGDVPIILGEFGLDTTLPGARDYIERVYGTAREMGAGVSYWSSDPGPWGPYLPDGTQTLLVDTLNKPYPRAVAGTPTEWSSTSDRLQLTIEPDAAITAPTEIYLPEAGFPGDVHVEGADVVGWDRQSRLLTVRTPADSGNVTVTVTPAA

SEQ ID NO: 8 - DNA, 2012 bp, Rhodococcus sp.
  Rhodococcus sp, strain C9 endoglycoceramidase, C9 EGCase
gggcccgaacggattccgccgcgagttcgccgggtcgacggacggcccggccgcgagggtctcggtctcgacgacggtcgacgcgggcggacgcaccctcgacctggtcgtgacgaacggaggaacccgggatgtgacggtcgtcgtcgacggccgcggtggaacgctgggtcccggcgcccgacgctcgtggacggtgccgtcgacggacggctggtaccggtgcgccgtgaccgtcgacgaggacacggacttccggcgcacgctggccggacacatcgagaacggcgaggacagcgtcagccaacccacctgacgcggcacctgccaccgtgcgggcacacggccgcacgaccgccatctgatccacacaacccgtaggaggagcgacagtgcgtccaggaggaacgacagtgcgtcgaacaagaatcgcgtcccttgccgtggcggggtcgctcgtactcggggccggtgtggccaccgcgcagagcagcttgccggccaccgggagtgactcgagcgagtggagcgcatcggcctacctgacggacgacgcgggccgatccctgatcctgcgtgggttcaacacggcatcgagcgcgaagagcaccccggacggcatgccgatcttcaccgagtccgacctggaccgcgagcacgccgacatgggaaccaacttcgtgcgcttcctgatctcctggcgttcggtggaacccgaaccgggacagtacgaccaggcgtatctggaccgggtcgagcagcgcgtcggctggtatgccgaacgcggctacaaggtcatgctcgacatgcaccaggacctctactccggcgcgatcacccccgacggcaagaccggcaacggcgcgccggcatgggcgacgtacatggacggtctccccgtcaacgagcgggacagctgggagctgtactacatcgagcccggcgtgatccgcgcgttcgacaacttctggaacaccaccggaaagcaccccgaactcgtcgaccactacgtgaatgcctggaaggccgtcgcggaccggttcgccgacaacgagactgtcgtcgcctacgacctgatgaacgagccgtggggcggatccttgcagggaccggcgttcgaggcaggaccactcacctcgatgtaccagcggaccaccgacgccatccgacaggtcgaccaggacagctgggtctgcgtcgccccgcaggctgtcggcgtcaaccagggcattccgagcgcactcggcacgatcgccgatccccgccagggcgctcggcgcatcgcctactgcccgcacctgtatcccctccccctcgacctcggtgacgggtactcggggttctcgaagaccctcaccgacgccaccatcgaaacctggcgcacgagcatcgaacacgtcgccgacaccgttctcgagggtgcaccggtgatcctcggagagttcgggctcgacaccaccctgcccggcgcccaggactacctcgatcgcgtctacaccgtcgctcgcgacatgggtgcgggtgtctcgtactggtcgagcgatcgcggtccctggggtccctacctggaggacgggacgcagaccatcctcgtcgacaccgtgaacaagccgtatccgcgggccgtggcgggcatgcccgtccggtggtcgtcgacctccgatcgactggacctgacgtaccgcaacgatcccgcggtgaccgcgcccaccgagatctaccttccggcagcaggattccccggcgacatcgccgtccagggggcggacgtggtcggatgggactcacagagtcggctcctgaccgttcggtccgcgcccgacgcgggtgaggtgaccgtgacggtgacgcccgcggcgtgaccccgtacctgcggccggccggtcaggccggccgcgggtggtgtcacatgtcgaggccgaggtccagcaccgtcaccgaatgggtgagagcgccgacggcgaggtagtcgacaccggtcgccgcgtagtcggccgcgacgcccagggtca

SEQ ID NO: 9 - protein, 482 aa, Rhodococcus sp.
  Rhodococcus sp, strain C9 endoglycoceramidase, C9 EGCase
MRRTRIASLAVAGSLVLGAGVATAQSSLPATGSDSSEWSASAYLTDDAGRSLILRGFNTASSAKSTPDGMPIFTESDLDREHADMGTNFVRFLISWRSVEPEPGQYDQAYLDRVEQRVGWYAERGYKVMLDMHQDLYSGAITPDGKTGNGAPAWATYMDGLPVNERDSWELYYIEPGVIRAFDNFWNTTGKHPELVDHYVNAWKAVADRFADNETVVAYDLMNEPWGGSLQGPAFEAGPLTSMYQRTTDAIRQVDQDSWVCVAPQAVGVNQGIPSALGTIADPRQGARRIAYCPHLYPLPLDLGDGYSGFSKTLTDATIETWRTSIEHVADTVLEGAPVILGEFGLDTTLPGAQDYLDRVYTVARDMGAGVSYWSSDRGPWGPYLEDGTQTILVDTVNKPYPRAVAGMPVRWSSTSDRLDLTYRNDPAVTAPTEIYLPAAGFPGDIAVQGADVVGWDSQSRLLTVRSAPDAGEVTVTVTPAA

SEQ ID NO: 10 - DNA, 1503 bp, Propionibacterium acnes
  Propionibacterium acnes, strain KPA171202 endoglycoceramidase (EGCa)
atgcgtcgaaagtctgccctcggatttgtagctttgtccctgttcgccacagggatgggcgttgccgcagcaacaccggcaactgcctcgccggcggatacggcagcgccagttcacgtcgacgcttcacggtggaccacccaggggcgttgggtgaccgacacccagcaccgcgtggtcatcacgcaggggatcaacgaggtcgccaagagcgccccctacgcccccgatgccgtcggtttcggtgaagacgacgcagccttcctcgaggcgcaggggttcaccagcgtccggctgggggtgctgtgggccggcgtcgagcctcggccgggcgtctacgacgacgcttacctggcccgggtcgaacgcaccgtgcggatcctcaacgcccacggcatcgccagtgtcctcgacttccatcaggacatggtcaacgagaagtaccagggggaggggtggcctgcctgggccgcgctcgaccacggcatgcccaacatcgtcaagacgggcttccccggcaactatttcctcaacgaggccgtcaaatactccttcgactccttctacgacaacaccaaggcctccgacggcatcggtgttgccgaccactacgccagcgcctggcgacatgtggccgagcatttccgaaacgtgcccggcgtgcagggctacgacctgttcaacgagccgttcccgggccaccgctacacgcggtgcctcacgcagctcggttgccgcgctgctgacgcgcgactgtcggccgtccagcagaagactgtcgacgcgatccgctcggtcgacaaggccaccactgtctggtacgagccgatgcagttcttcaatataggtgtcgggaccaacgtccggctcacgggatccaacctggggttgagcttccacgactactgcaccagccaggccaccctccactcctatgtcgggtgcactgcgcccgacaaccgggtcttcactaacgcagagaagcattcacgtcagaccgggtcggggctgatgctcaccgagttcggcgccatcacgacccccgcggtgatcacgtcccagatggacctggcagctcgcaaccgggtcggcgtccagtggtgggcctacactgccggtgatcccaccacagccggcccgggcaccgagcaagccctcgtcgacgacccagctcggccaccccaggggaccaacgtcgaaagcgccaagctgacgctgatcgccgttccccacccggaccgtgtcgcgggcaccccatccgcgtaccaccacgaccggtcccgacgcgtgttcaccatgacctggaccgcccagcggcccgacgggtcgcgcgcggaggagtcggacgagacgactgtggtggtccctgccatctcagcgccccacgggtacgacgtgcaggcatccggcgcccacgtcacctcccacccaggcgaccgggtggcgcggttgcacctcaaccaaggcagtgccacggcgaaggtcacgatcaccctgcgctaa

SEQ ID NO: 11 - protein, 500 aa, Propionibacterium acnes
  Propionibacterium acnes, strain KPA171202 endoglycoceramidase (EGCa)
MRRKSALGFVALSLFATGMGVAAATPATASPADTAAPVHVDASRWTTQGRWVTDTQHRVVITQGINEVAKSAPYAPDAVGFGEDDAAFLEAQGFTSVRLGVLWAGVEPRPGVYDDAYLARVERTVRILNAHGIASVLDFHQDMVNEKYQGEGWPAWAALDHGMPNIVKTGFPGNYFLNEAVKYSFDSFYDNTKASDGIGVADHYASAWRHVAEHFRNVPGVQGYDLFNEPFPGHRYTRCLTQLGCRAADARLSAVQQKTVDAIRSVDKATTVWYEPMQFFNIGVGTNVRLTGSNLGLSFHDYCTSQATLHSYVGCTAPDNRVFTNAEKHSRQTGSGLMLTEFGAITTPAVITSQMDLAARNRVGVQWWAYTAGDPTTAGPGTEQALVDDPARPPQGTNVESAKLTLIAVPHPDRVAGTPSAYHHDRSRRVFTMTWTAQRPDGSRAEESDETTVVVPAISAPHGYDVQASGAHVTSHPGDRVARLHLNQGSATAKVTITLR

SEQ ID NO: 12 - DNA, 1575 bp, Propionibacterium acnes
  Propionibacterium acnes, strain KPA171202 endoglycoceramidase (EGCb)
atgtatcaccattcatggcattccccggatgcacgacgccgaggcgtcacccggtgggcgaccaccttcattgctgcccttactgccgcctgcatggcacagatgcctgcacaggcctcgccccataccagcgacgccgctccccacatcgcaacgtcaaagaccatcaccgacgccggccccatcgggcagtccggccgttggtacaccgacggtcagggtcgcgctatcctcaccgccggcgtcaacatggtctctaaacgtcacccatacagtcccgaagccgatggattcgatgacgccgacgctgcctggttacagaagaacggcttcgattcggtgcgcctgggagtcatatggaagggggtcgagcccaagcccggagagtacgacgacgcctacctggccagcatcacccgcacagtaagaacacttcgcgctcacggcataatgaccctcttggacgctcaccaggacatgtataacgagaagttcgagggtgagggagcccccgactgggccgttctcgacaagggagcaccgaatctgctcaaggttggcttccccgccaaccaggtcttcaacctcggactcatcaaggcttacgacagtttcctggacaatgccaagggcccgggcggagtgggcttgcaggatcgttacgcggccatgtggaagcacgtcgcacaggtcgtcgggcaggaacccggcgtcatgggatacgacattatcaacgagccttggccgggacatcactaccccatctgctacgttgccttcggctggtgcggccgagcgatggtgtccttggacaccttgtacgagaaagtcggcagagccatcacctcggtcgaccccgacggcatcgtcacctacgagccctactcaacgtggaacatggggctggacagccgcccagcccgcccatcctcaccgaaggctgccatttcttggcacgtctactgccccatgaacgcaatcttcggctcctacgtcgggtgcaatctccccgacactcgcaccttccacaacgccgaccaggcagcccagttcaacaactcagcctccttgctcagtgaattcggggccaccaaagaccccggcactctcatgggggtcacatccaaggctcgcgcccatctggtcggctggctgtactggacgtacaacggaaactccgacccgacaacccagaatgctgcagacgaggagctcgtccgtcatatcaaccgtccgggacctgtcaccgacgaacaagtggaccacaccaagctcgccattctggcggtaccgcacctgcgcgccgctgcgggcaccccgacctcgacgacctgggaccagtccacccggacgtaccaggccacgtggacggctaaacgtgtcgccggtgacggtgacttcgcggcaggatccgtctccgagatcgccgtcccggctatccactaccccaatggttacaaggtcgaggtgaagggcgccaaggtcatttccaaagccggagacacacgcctgcaggtcagctccaccggagaaggcccggtaagcgtcaccatcacccctgccggtcaggcctaa

SEQ ID NO: 13 - protein, 524 aa, Propionibacterium acnes
  Propionibacterium acnes, strain KPA171202 endoglycoceramidase (EGCb)
MYHHSWHSPDARRRGVTRWATTFIAALTAACMAQMPAQASPHTSDAAPHIATSKTITDAGPIGQSGRWYTDGQGRAILTAGVNMVSKRHPYSPEADGFDDADAAWLQKNGFDSVRLGVIWKGVEPKPGEYDDAYLASITRTVRTLRAHGIMTLLDAHQDMYNEKFEGEGAPDWAVLDKGAPNLLKVGFPANQVFNLGLIKAYDSFLDNAKGPGGVGLQDRYAAMWKHVAQVVGQEPGVMGYDIINEPWPGHHYPICYVAFGWCGRAMVSLDTLYEKVGRAITSVDPDGIVTYEPYSTWNMGLDSRPARPSSPKAAISWHVYCPMNAIFGSYVGCNLPDTRTFHNADQAAQFNNSASLLSEFGATKDPGTLMGVTSKARAHLVGWLYWTYNGNSDPTTQNAADEELVRHINRPGPVTDEQVDHTKLAILAVPHLRAAAGTPTSTTWDQSTRTYQATWTAKRVAGDGDFAAGSVSEIAVPAIHYPNGYKVEVKGAKVISKAGDTRLQVSSTGEGPVSVTITPAGQA

SEQ ID NO: 14 - DNA, 1730 bp, Cyanea nozakii
  Cyanea nozakii jelly fish endoglycoceramidase (EGCase)
ggcgatttgcaatggctgaaacacaaccattggtgtttgtcttgatgagcatttcagctattttaacggcaggacttccaataaacgatgatgcatcattgttgataagcgtcaatcctgaaacacaacagttggttgatagtttggggagagagagattttttcatggaacgaacgttgttgtcaaacataaaccttatcatccatcagttgagggttatgacaatacgtctttctcagaagttgatatgaagattttgcaagatcttggcctcaatacaattcgccttggtatgatgctgccaggctacgtgcctacccgaggtaattacaatgaaacatacttgaagatcatacaggaaattgtatcaaaggcagctaaatatggcatttatactttactggatatgcaccaggatgttatgtctgcaaagttttgcgttgaaggatttcctgattgggctgttaatacaggcaatgcagacaatttcccttttccacttgaagacaaataccccctgaatctgcagactggatacccttatccaaaagactgtgcaaagcatgcctggggggactactacttcacggaagcagccgccgcagctttccagaacttctacaataacactgacgggctattagatgcatgggcggacttctggaagaaaacagcacagggtttcaaagattataaaagtgtcattggatatgaacttattaatgaaccatttgctggcgatatatacagggatccttcactcatgattcctggcgttgcggacgaaagaaacctcgcgccagcctatgacgtcatccataaagccattcgtacggtggatgaacaacacagcatatttttcgagggcgtaacgtgggattatttcgcggcgggattcagtaaagtaccaggcggtgacgcataccgtaatcggagcgttttaagctatcattattacgagcctccagatttcaataagaagtttcagttcgaggtgcgtatggaagatcttaggcgtttaaaatgtggcggtttcttgaccgaacttcttacggttggcgatacggcgaaagatatgagcgatatgctcgaacttttcgacatttgcgatcaacataagcagtcctggatgggatggctatacaaatcctacggttgctacaagcaacatctgggctgtctaacggactctatgcatgacgaaacaggacatttacgcgatatcgtccttcaaaacactactcgcacctacccgcaagctgtcgcaggacacacaattggatataagtttgacaggattacgaaaaagttcgatttgagtttcgtcgttactgcagattgtcgaagcacggagtctatcgtctacttcaacaaagatttacattactcgaatggttacgacgttacggtttttccgaaagattccgttacgtggaagcaagtagagaagaaaataatcatcaaccattcgcaaaagctttctgctggcacgactgtgactttctctctcgttgctaagtagctattgccatggaaacaaatattctgctgttggtgattcaaatctgaaaaggactgcgtattatatcagtgtcatgatttatattaaaacgaggctaatccaaaatggctgggtagattttgttgctaatagtgaacaatagtgaaaaccaagatatgccataaaaagtttgttttaaaaaaaaaaaaaaaaaaaaaaaaaaaaaaa

SEQ ID NO: 15 - protein, 503 aa, Cyanea nozakii
  Cyanea nozakii jelly fish endoglycoceramidase (EGCase)
MAETQPLVFVLMSISAILTAGLPINDDASLLISVNPETQQLVDSLGRERFFHGTNVVVKHKPYHPSVEGYDNTSFSEVDMKILQDLGLNTIRLGMMLPGYVPTRGNYNETYLKIIQEIVSKAAKYGIYTLLDMHQDVMSAKFCVEGFPDWAVNTGNADNFPFPLEDKYPLNLQTGYPYPKDCAKHAWGDYYFTEAAAAAFQNFYNNTDGLLDAWADFWKKTAQGFKDYKSVIGYELINEPFAGDIYRDPSLMIPGVADERNLAPAYDVIHKAIRTVDEQHSIFFEGVTWDYFAAGFSKVPGGDAYRNRSVLSYHYYEPPDFNKKFQFEVRMEDLRRLKCGGFLTELLTVGDTAKDMSDMLELFDICDQHKQSWMGWLYKSYGCYKQHLGCLTDSMHDETGHLRDIVLQNTTRTYPQAVAGHTIGYKFDRITKKFDLSFVVTADCRSTESIVYFNKDLHYSNGYDVTVFPKDSVTWKQVEKKIIINHSQKLSAGTTVTFSLVAK

SEQ ID NO: 16 - DNA, 1730 bp, Cyanea nozakii
  Cyanea nozakii jelly fish endoglycoceramidase (EGCase-P)
ggcgatttgcaatggctgaaacacaaccattggtgtttgtcttgatgagcatttcagctattttaacggcaggacttccaataaacgatgatgcatcattgttgataagcgtcaatcctgaaacacaacagttggttgatagtttggggagagagagatttttccatggaacgaacgttgttgtcaaacataaaccttatcatccatcagttgagggttatgacaatacgtctttctcagaagttgatatgaagattttgcaagatcttggcctcaatacaattcgccttggtatgatgctgccaggctatgtgcctacccgaggtaattacaatgaaacatacttgaagatcatacaggaaattgtatcaaaggcagctaaatatggcatttatactttactggatatgcaccaggatgttatgtctgcaaagttttgcgttgaaggatttcctgattgggctgttaatacaggcaatgcagacaatttcccttttccacttgaagacaaataccccctgaatccgcagactggatacccttatccaaaagactgtgcaaagcatgcctggggggactactacttcacggaagcagccgccgcagctttccagaacttctacaataacactgacgggctattagatgcatgggcggacttctggaagaaaacagcacagggtttcaaagattataaaagtgtcattggatatgaacttattaatgaaccatttgctggcgatatatacagggatccttcactcatgattcctggcgttgcggacgaaagaaatctcgcgccagcctatgacgtcatccataaagccattcgtacggtggatgaacaacacagcatatttttcgagggcgtaacgtgggattatttcgcggcgggattcagtaaagtaccaggcggtgacgcataccgtaatcggagcgttttaagctatcattattacgagcctccagatttcaataagaagtttcagttcgaggtgcgtatggaagatcttaggcgtttaaaatgtggcggtttcttgaccgaacttcttacggttggcgatacggcgaaagatatgagcgatatgctcgaacttttcgacatttgcgatcaacataagcagtcctggatgggatggctatacaaatcctacggttgctacaagcaacatctgggctgtctaacggactctatgcatgacgaaacaggacatttacgcgatatcgtccttcaaaacactactcgcacctacccgcaagctgtcgcaggacacacaattggatataagtttgacaggattacgaaaaagttcgatttgagtttcgtcgttactgcagattgtcgaagcacggagtctatcgtctacttcaacaaagatttacattactcgaatggttacgacgttacggtttttccgaaagattccgttacgtggaagcaagtagagaagaaaataatcatcaaccattcgcaaaagctttctgctggcacgactgtgactttctctctcgttgctaagtagctattgccatggaaacaaatattctgctgttggtgattcaaatctgaaaaggactgcgtattatatcagtgtcatgatttatattaaaacgaggctaatccaaaatggctgggtagattttgttgctaatagtgaacaatagtgaaaaccaagatatgccataaaaagtttgttttaaaaaaaaaaaaaaaaaaaaaaaaaaaaaaa

SEQ ID NO: 17 - protein, 503 aa, Cyanea nozakii
  Cyanea nozakii jelly fish endoglycoceramidase (EGCase-P)
MAETQPLVFVLMSISAILTAGLPINDDASLLISVNPETQQLVDSLGRERFFHGTNVVVKHKPYHPSVEGYDNTSFSEVDMKILQDLGLNTIRLGMMLPGYVPTRGNYNETYLKIIQEIVSKAAKYGIYTLLDMHQDVMSAKFCVEGFPDWAVNTGNADNFPFPLEDKYPLNPQTGYPYPKDCAKHAWGDYYFTEAAAAAFQNFYNNTDGLLDAWADFWKKTAQGFKDYKSVIGYELINEPFAGDIYRDPSLMIPGVADERNLAPAYDVIHKAIRTVDEQHSIFFEGVTWDYFAAGFSKVPGGDAYRNRSVLSYHYYEPPDFNKKFQFEVRMEDLRRLKCGGFLTELLTVGDTAKDMSDMLELFDICDQHKQSWMGWLYKSYGCYKQHLGCLTDSMHDETGHLRDIVLQNTTRTYPQAVAGHTIGYKFDRITKKFDLSFVVTADCRSTESIVYFNKDLHYSNGYDVTVFPKDSVTWKQVEKKIIINHSQKLSAGTTVTFSLVAK

SEQ ID NO: 18 - DNA, 1856 bp, Hydra magnipapillata
  Hydra magnipapillata hydrozoan endoglycoceramidase (EGCase)
atatattaaaaaaaaaaaatgataagcgtcgcacttattatactttttcttgcaaaagttatttccggaaaatcggatgattttatatctgtaaaccctgaaacaaatatgcttattgatggctatgggcgagaaagattttttcacggtaccaatgttgtggtgaagcattttccttttcatcctgaaactacagggtttaacaaagacacgttttctgaagatgacatgaaaattctacagaagtttggattaaactcaattcgattaggaatgatgctacctggatatgtgccaaaaagagaggaatataatgaaacttatataaaagttatacaaagtattgtcactacagctgcaaagtatggtatttacacattgttagacatgcatcaagatgttttttcaccaaaattttgtgtagaaggcatgcctgattggatagttaacacacaaggagcaaaagattttccaatgccacttcataaaccgttcaatttggatcctaaaacaggatatccataccctgaggattgcgccaagttttcatgggcagactattattttactgaagcagcaggacaagcttttcaaaatctttacgacaatgttgatggactgcgtgacgaatgggcacaattttggaaaaaaactgctgatgtttttaaagaagaacctagcgttattggatatgaactcataaacgaaccgttttgtggcaatgtatttaaacacccgacattgctgattcccggtgttgccgattatctcaacctacaaccaacatatgacgcattacaaaaagctatacgtcaagttgatgaagaacataacatattttttgaaggagttacatgggacttttttgaagttggttttactgaagttcctggcggtaaacagtatcaaaatcggagcgttcttagttatcattattatgagccgccagacttttctaaaaaactaaattttgaagctcgtttgcttgatcttaaacgattgaaatgtggtggatttcttactgaaatgtttacagttggaacagattttaacagcatgtttgaaatgtttgatttatgcgataaattcaagcaaagttggcatggatggatgtataaatcatacgggtgtatagagcaaaacctgggttgtttgaatatgtcttctccaggtaaagaatctattcaaattgcgaacacttcaagaacgtatccacaggcggtggctgggcgtacgcaatcctacgcatttgacataaagactaaagtattcacattggtatacgaaactgttggcagttgcaaaagtggtagaaccattgtttactttaataaaaatcttcattatcctaacggatatcgctatgagataaatccaaatttcaaagtaacccccagtgaaaatgaatactttctttatttagatgaagttaataaagtaccaaacaccgttgtgacatttaaactttttccactcagctttactgatagtgaagatattcatccagtaacggtgatgggtgataaacatctatcagaaaatcataatgaaaatgaaaaaaaaaaaaagtgaaaattatatttgaaaaaaataattcgactttaaacacattttaaaaattacttattataaaaacgtttttaaatattttttaatgtaaaattttaaaaatcaatgaagttaatataagctttaaataacatttatggtatattatttataaattgtaacatttaaagcacaggtcagcaaaataatttttttttggtttttaagatatcaggtatgattttgtataatttggtgtgctgaatttgagaataacattttatgaaaaaaaaaaaaaaaaaaaaaaa

SEQ ID NO: 19 - protein, 517 aa, Hydra magnipapillata
  Hydra magnipapillata hydrozoan endoglycoceramidase (EGCase)
MISVALIILFLAKVISGKSDDFISVNPETNMLIDGYGRERFFHGTNVVVKHFPFHPETTGFNKDTFSEDDMKILQKFGLNSIRLGMMLPGYVPKREEYNETYIKVIQSIVTTAAKYGIYTLLDMHQDVFSPKFCVEGMPDWIVNTQGAKDFPMPLHKPFNLDPKTGYPYPEDCAKFSWADYYFTEAAGQAFQNLYDNVDGLRDEWAQFWKKTADVFKEEPSVIGYELINEPFCGNVFKHPTLLIPGVADYLNLQPTYDALQKAIRQVDEEHNIFFEGVTWDFFEVGFTEVPGGKQYQNRSVLSYHYYEPPDFSKKLNFEARLLDLKRLKCGGFLTEMFTVGTDFNSMFEMFDLCDKFKQSWHGWMYKSYGCIEQNLGCLNMSSPGKESIQIANTSRTYPQAVAGRTQSYAFDIKTKVFTLVYETVGSCKSGRTIVYFNKNLHYPNGYRYEINPNFKVTPSENEYFLYLDEVNKVPNTVVTFKLFPLSFTDSEDIHPVTVMGDKHLSENHNENEKKKK

SEQ ID NO: 20 - DNA, 1830 bp, Schistosoma japonicum
  Schistosoma japonicum blood fluke similar to endoglycoceramidase (EGCase), SJCHGC02838 protein
agaattgtcgatagccgagagtagatctatagtataatatagtgttcattgaaataattgtcactaattcaactactaattcattaacttttacaataatacttagtctggttattattaccaaacgtagttattattccatgtggtcaatattcatcttgacatttctaatctggacatcagttcagacaaaacagatcccactgagcaaaatacatctcaattcagatggactattcactgattctcgaggattcattaaattatttagagggtttaacaatgtgcataaacattttccatggtataatgtaaattctacgaatatcacacaattagaaatgtttaaaaattggggtttgaatgttgttcgattaggtgtaatgtggagtggagtgaagccgacaatatcaatagtgaataccacatacttagatgtgattgagaatgtgattgatttatatgctgattatgggatttatgtaatattggatatgcatcaagatgtattgtcatcgttgtatggtctttatgatggcattccactatggttaattgaaaaatttaagagaccacctcatcatttacaatatccctggccatataagaaaaagccagatttttgggtgatgtcttatttaacttatgaatgtgctaatggagcccagcaattgtataataatgtgtcgggtgcatggaatcattggggtgaattttgggaaatagtggctagacgatttggtggaaagtcaaatgtgcttggttatgaattgataaatgaaccaccaccaggaaacttttataccaatccacttcgaggtcttccaggttatgctggtcgatataacttgcaaccggtttatgattatctcgttaagagaatacgcaaatacgacaattcgacactgatattctatgaaccagttacatatggagtatttacgccagtgagatcatcaggatggttaggaactggattcgatcgcgtccctggagcccatcgtgacaaatcggcaccaagtaaaagtgttctatcttatcattattactgttggatactacaaactgatgcacaaaacacgacaatgccattctggaagaaagttatctgtgacaggctcctcttgcctaacgtcatctccaatgcaatcagagcaacaaagtcaactggaggtggccgatttctaactgaattcggtttatgtggagatgacgggaatccacgtagtgtgaatacaattgaatgtaataatatattaaatgaagctgataaacattttgaatcatggacctactgggacagtaatctcttagatttgtcaggaaatcctatagtaactgaggtgaaatcattcattcgtccgtatccacattcaataagaggagtatttcggaagcaacagttcgatcataaaacaggggattttcacctctccttcattgctaacacaaccaaagagcagaacaatgagaagcagacgttgatcgcagagatttacataccgagatctgttcattatcccaatggattttccatgagtgtgaaaccggacaatttaagcacgaagatgaatgagaatatgatgtatgtatacttaccaagtggtgtcagtaatgcgagtgtgtttgttcgaatcgaaatagtgagaaaatcgatcgagtgaactattctaattgtggtggctatccgctgaactaaatgtcattgatgttattcatatgttatctgtgttattgaattcaacaagttgtgtgtttgtttatttctattgatttctactgttccgacttttttatttttaaatatatcagtcatccataatcatccat

SEQ ID NO: 21 - protein, 507 aa, Schistosoma japonicum
  Schistosoma japonicum blood fluke similar to endoglycoceramidase (EGCase), SJCHGC02838 protein
MWSIFILTFLIWTSVQTKQIPLSKIHLNSDGLFTDSRGFIKLFRGFNNVHKHFPWYNVNSTNITQLEMFKNWGLNVVRLGVMWSGVKPTISIVNTTYLDVIENVIDLYADYGIYVILDMHQDVLSSLYGLYDGIPLWLIEKFKRPPHHLQYPWPYKKKPDFWVMSYLTYECANGAQQLYNNVSGAWNHWGEFWEIVARRFGGKSNVLGYELINEPPPGNFYTNPLRGLPGYAGRYNLQPVYDYLVKRIRKYDNSTLIFYEPVTYGVFTPVRSSGWLGTGFDRVPGAHRDKSAPSKSVLSYHYYCWILQTDAQNTTMPFWKKVICDRLLLPNVISNAIRATKSTGGGRFLTEFGLCGDDGNPRSVNTIECNNILNEADKHFESWTYWDSNLLDLSGNPIVTEVKSFIRPYPHSIRGVFRKQQFDHKTGDFHLSFIANTTKEQNNEKQTLIAEIYIPRSVHYPNGFSMSVKPDNLSTKMNENMMYVYLPSGVSNASVFVRIEIVRKSIE

SEQ ID NO: 22 - protein, 509 aa, Dictyostelium discoideum
  Dictyostelium discoideum, strain AX4 slime mold putative endoglycoceramidase (EGCase), hypothetical protein DDBDRAFT_0190788
MNKKKQIITTITLLSFINLFSIVNAIIKVNPANQFFIDQYNRVRLFHGVNVVYKIPPFHPSLEGFDPVTSFSSQDIENLVEWGFNAVRLGVMWPGVEPVKDEYNQTYLDVMSKLVSEMEDNEIYTLIDFHQDLLSRKYCGEGLPDWIVSNDTNDSFPSPVAHSYPKNNESYPSLDQCLNKDFGVYYFSEDVNREFQNLYDNVNGVQDKFIDYWRQVVNTFKSYDTVLGYEIINEPWGGDIYQNPEYLLKLGYADSKNLLPLYQAVNNAIRELDDQHCVYYEKALTDLFHSYFPSGTPGGVQYNDRQVLSYHIYCATDRDGNPRHEYVCDGEDDIFLVSAMKDLKQTGGGGFMTEFGAVSNGTNSIEMLNYLTGSADKYLQSWTYWQLKYYNDITTAGSTESLYLPNGELDIPKITALSRTYAQAIAGVPLSMSFNPANSDFSFSYNINTTITQPTQIYLNQDIYYPNGFTTNIITGTATVSIPQKNLIYILPNSNTINQSTITITILKK

SEQ ID NO: 23 - protein, 647 aa, Streptomyces avermitilis
  Steptomyces avermitilis, strain MA-4680 putative endoglycoceramidase (EGCase)
MRKNAKLTHESEVLTFHRSARTVVDMSKLRARLLGVLVSLTGLLGATGAQPAAADSLPDSLWFDASASAAFTVQNGRFSDGLGREVVLRGYNVSGETKLEENSGLPFASVADARKSATALRTLGGGNSVRFLLSWAHAEPVRGQVDTAYLAAATAQMRAFLDAGIRVFPDFHQDLYSRYLFNSGSWYTGDGAPEWAVDAGDYPAESCGICLFWGQNITQNGAVTQASHDFWHNAYGVQDAFLATAQATMAYIQQNLSADEFNGVVGFDPYNEPHAGTYDSGETSRTWEQNVLWPFYKKFRARMDAAGWQTKPAFIEPNLFWNANIDFQKQEGGLLDAGTLGPRYVLNTHFYDQKAISGVLMWGKAADGQYATDFGKVRDRAAGAGTAAVVSEFGHPLSGSVSDKAPTVVKAMYQALDSRLPGSTWWSDPTGSGPVLSGAQWQWDIYNGRHHELENGNPDKVLTSGDAWNDEDLSAVSLNDSGTAVLRQDARLLDRLYPSATAGATVAFTYEDRSRDGSTTLTWNPVPSSLPNVSRLVGSGQYGLLVWRSNGSTAPTELHLPASFPAASTTVVSDLGTTSGLPAYTRTTPVGHAAEPGGTGSHRLLLTAADSGTVHYALVTNGATAPSAGLLSAARAELSSWAATKVG

SEQ ID NO: 24 - protein, 654 aa, Leptospira interrogans
  Leptospira interrogans, serovar Copenhageni, strain Fiocruz L1-130 spirochete putative endoglycoceramidase (EGCase), hypothetical protein LIC20191
MEELFVKNGHFASKEGAIYQLRGVNLSGSAKLPLKPDGTTHFDQTTTFDNHKNVSFVGRPLKEDQAEEHFDRLRKWGFNFLRFLITWEAIEHKGPGKYDNEYIDYVERMVSLAAKKGFYLFIDPHQDVWSRFTGGDGAPGWTLEELGMNISKIRNSETAIVHHHQGKNYRRMSWPLNYQKYSCATMFSLFFGGKEFAPDTKIDGRNVQDFLQDHYIDSVLKIVRKLKKYKNVIGFDTLNEPSPGWIGKKNLGEFDGFGFGKVVKSSPFQEMYLSEGRAVSAAQAYMLGFWSLPFGKVRLNPEGVPLWERGHQCIWRNHGVWDYDPNGAPMMLKPEYFYKKNGRKYEFYSDFMYPFIKKFKERVQKLENRFHIFIESDPSKLELEWKEIPKKNQGSVINATHWYDISVLMLKRYLPWFGVHVFKQKPIFGKENIDNAYEETIRMIREMSEKKMGNCPTVIGETGIPMDLNHRVAYLKNDYGVLEKALDRIMKAVEKNFVNLALWNYTPDHTHSLGDRWNEEDLSIYSQDTPSSYDEDGGRAVRAFSRPYPIRTKGFPVALTFDMERSLFKYAFRQEGDLFPETEIFIPEIHYKKGFEVLVNAGTYQYDFRSRVLKFKGEKGILDYGITVYPSKKSLSREQDRTKVVPKTQKRKTQ

SEQ ID NO: 25 - protein, 770 aa, Neurospora crassa
  Neurospora crassa, strain OR74A fungus putative endoglycoceramidase (EGCase), hypothetical protein NCU02233.1
MAGFRLTIENGSFRDVHGRQITLRGINVAGDAKYPNKPEQPSHVGENFFDGDNVKFTGRPFPKEEAHLHFSRLKRFGYNTIRYVFTWEAIEAAGPGIYDEEWIQHTIDVLRVAKRYGFYIFMDPHQDVWSRFSGGSGAPMWTLYAAGLNPQSFAATEAAIVHNVYPEPHNFPKMIWSTNYYRLAAATMFTLFFAGRDFAPKCIIDGVNIQDYLQDHFLRACAHLAQRIHEAGDIENDVVFGWESLNEPNKGMIAYEDISVIPKEQNLKKGTCPTIWQTILTGSGRAVEVDTWDMGGMGPYKVGRALIDPSGEQAWLPADYDESRYGYKRDPGWKLGQCIWAQHGVWDPATDSLLKKDYFGKHPATGEHVDYPYFSNRYFMDFFRKYRDTIRSIHPNAIILLQGPTMELPPKIIGTPDGDDPLLVYAPHWYDGITLMTKKWNRVWNVDVIGILRGKYWSPAFGIKIGETAIRNCFKNQHATMRQEGLDYIGNHPCVMTEFGIPYDMDDKNAYKTGDYSSQSAAMDANHYGVEGAGLEGYTLWLYMTKNDHELGDQWNGEDLSIFSVDDKLLPESPVPKSHSRDGSSSSIATPTGTKDDDLDDDSSVTPANIKRTLTNPSISSVSTQRQPELTNSPGYRAAEAYVRPAPIATAGTVKKYGFDLRSCQFHVTIQAPEAAKPDTPTVVFLPDYHFPKDACQVEVSSGKWEIRSDEEETTPLQKLRWWHGEGEQTLRVTGVVKQVNGNSSEGAEVGYYDQVFNQAKGFLDACVIM

SEQ ID NO: 26 - protein, 490 aa, Artificial Sequence
  Description of Artificial Sequencemutant endoglycoceramidase (E233A) derived from Rhodococcus sp, strain M-177 endoglycoceramidase II, endoglycoceramide synthase
MRRTRLVSLIVTGSLVFGGGVAAAQSSLAASGSGSGSGTALTPSYLKDDDGRSLILRGFNTASSAKSAPDGMPQFTEADLAREYADMGTNFVRFLISWRSVEPAPGVYDQQYLDRVEDRVGWYAERGYKVMLDMHQDVYSGAITPEGNSGNGAGAIGNGAPAWATYMDGLPVEPQPRWELYYIQPGVMRAFDNFWNTTGKHPELVEHYAKAWRAVADRFADNDAVVAYDLMNAPFGGSLQGPAFEAGPLAAMYQRTTDAIRQVDQDTWVCVAPQAIGVNQGLPSGLTKIDDPRAGQQRIAYCPHLYPLPLDIGDGHEGLARTLTDVTIDAWRANTAHTARVLGDVPIILGEFGLDTTLPGARDYIERVYGTAREMGAGVSYWSSDPGPWGPYLPDGTQTLLVDTLNKPYPRAVAGTPTEWSSTSDRLQLTIEPDAAITAPTEIYLPEAGFPGDVHVEGADVVGWDRQSRLLTVRTPADSGNVTVTVTPAA

SEQ ID NO: 27 - protein, 490 aa, Artificial Sequence
  Description of Artificial Sequencemutant endoglycoceramidase (E233S) derived from Rhodococcus sp, strain M-177 endoglycoceramidase II, endoglycoceramide synthase
MRRTRLVSLIVTGSLVFGGGVAAAQSSLAASGSGSGSGTALTPSYLKDDDGRSLILRGFNTASSAKSAPDGMPQFTEADLAREYADMGTNFVRFLISWRSVEPAPGVYDQQYLDRVEDRVGWYAERGYKVMLDMHQDVYSGAITPEGNSGNGAGAIGNGAPAWATYMDGLPVEPQPRWELYYIQPGVMRAFDNFWNTTGKHPELVEHYAKAWRAVADRFADNDAVVAYDLMNSPFGGSLQGPAFEAGPLAAMYQRTTDAIRQVDQDTWVCVAPQAIGVNQGLPSGLTKIDDPRAGQQRIAYCPHLYPLPLDIGDGHEGLARTLTDVTIDAWRANTAHTARVLGDVPIILGEFGLDTTLPGARDYIERVYGTAREMGAGVSYWSSDPGPWGPYLPDGTQTLLVDTLNKPYPRAVAGTPTEWSSTSDRLQLTIEPDAAITAPTEIYLPEAGFPGDVHVEGADVVGWDRQSRLLTVRTPADSGNVTVTVTPAA

SEQ ID NO: 28 - protein, 490 aa, Artificial Sequence
  Description of Artificial Sequencemutant endoglycoceramidase (E233G) derived from Rhodococcus sp, strain M-177 endoglycoceramidase II, endoglycoceramide synthase
MRRTRLVSLIVTGSLVFGGGVAAAQSSLAASGSGSGSGTALTPSYLKDDDGRSLILRGFNTASSAKSAPDGMPQFTEADLAREYADMGTNFVRFLISWRSVEPAPGVYDQQYLDRVEDRVGWYAERGYKVMLDMHQDVYSGAITPEGNSGNGAGAIGNGAPAWATYMDGLPVEPQPRWELYYIQPGVMRAFDNFWNTTGKHPELVEHYAKAWRAVADRFADNDAVVAYDLMNGPFGGSLQGPAFEAGPLAAMYQRTTDAIRQVDQDTWVCVAPQAIGVNQGLPSGLTKIDDPRAGQQRIAYCPHLYPLPLDIGDGHEGLARTLTDVTIDAWRANTAHTARVLGDVPIILGEFGLDTTLPGARDYIERVYGTAREMGAGVSYWSSDPGPWGPYLPDGTQTLLVDTLNKPYPRAVAGTPTEWSSTSDRLQLTIEPDAAITAPTEIYLPEAGFPGDVHVEGADVVGWDRQSRLLTVRTPADSGNVTVTVTPAA

SEQ ID NO: 29 - protein, 490 aa, Artificial Sequence
  Description of Artificial Sequencemutant endoglycoceramidase (E233D) derived from Rhodococcus sp, strain M-177 endoglycoceramidase II, endoglycoceramide synthase
MRRTRLVSLIVTGSLVFGGGVAAAQSSLAASGSGSGSGTALTPSYLKDDDGRSLILRGFNTASSAKSAPDGMPQFTEADLAREYADMGTNFVRFLISWRSVEPAPGVYDQQYLDRVEDRVGWYAERGYKVMLDMHQDVYSGAITPEGNSGNGAGAIGNGAPAWATYMDGLPVEPQPRWELYYIQPGVMRAFDNFWNTTGKHPELVEHYAKAWRAVADRFADNDAVVAYDLMNDPFGGSLQGPAFEAGPLAAMYQRTTDAIRQVDQDTWVCVAPQAIGVNQGLPSGLTKIDDPRAGQQRIAYCPHLYPLPLDIGDGHEGLARTLTDVTIDAWRANTAHTARVLGDVPIILGEFGLDTTLPGARDYIERVYGTAREMGAGVSYWSSDPGPWGPYLPDGTQTLLVDTLNKPYPRAVAGTPTEWSSTSDRLQLTIEPDAAITAPTEIYLPEAGFPGDVHVEGADVVGWDRQSRLLTVRTPADSGNVTVTVTPAA

SEQ ID NO: 30 - protein, 490 aa, Artificial Sequence
  Description of Artificial Sequencemutant endoglycoceramidase (E233Q) derived from Rhodococcus sp, strain M-177 endoglycoceramidase II, endoglycoceramide synthase
MRRTRLVSLIVTGSLVFGGGVAAAQSSLAASGSGSGSGTALTPSYLKDDDGRSLILRGFNTASSAKSAPDGMPQFTEADLAREYADMGTNFVRFLISWRSVEPAPGVYDQQYLDRVEDRVGWYAERGYKVMLDMHQDVYSGAITPEGNSGNGAGAIGNGAPAWATYMDGLPVEPQPRWELYYIQPGVMRAFDNFWNTTGKHPELVEHYAKAWRAVADRFADNDAVVAYDLMNQPFGGSLQGPAFEAGPLAAMYQRTTDAIRQVDQDTWVCVAPQAIGVNQGLPSGLTKIDDPRAGQQRIAYCPHLYPLPLDIGDGHEGLARTLTDVTIDAWRANTAHTARVLGDVPIILGEFGLDTTLPGARDYIERVYGTAREMGAGVSYWSSDPGPWGPYLPDGTQTLLVDTLNKPYPRAVAGTPTEWSSTSDRLQLTIEPDAAITAPTEIYLPEAGFPGDVHVEGADVVGWDRQSRLLTVRTPADSGNVTVTVTPAA

SEQ ID NO: 31 - DNA, 1401 bp, Artificial Sequence
  Description of Artificial Sequencemutant endoglycoceramidase (His E351S) derived from Rhodococcus sp, strain M-177 endoglycoceramidase II, endoglycoceramide synthase
catatgggatccagcggaagcggtagcggttcgggtaccgcgctgacaccttcatatctgaaggatgatgacgggcggagcctcattcttcgtggatttaatacggcctcatctgcaaaaagtgcccctgacggcatgccacagttcactgaagcagatttggcgcgtgaatatgcggacatgggtactaattttgtacgttttctgatctcttggcgctcggtggaaccggctcctggcgtatatgatcaacagtacctggatcgtgtagaagaccgtgtaggttggtacgcagagcgtggttataaagttatgctggacatgcatcaagacgtgtactcgggggccattactccggaaggcaatagtggtaatggcgcaggtgcgattggtaatggggcaccggcgtgggccacctatatggatggtctgccagtggaaccccaaccccgctgggaactgtattacatccagccaggcgtgatgcgggcgtttgataatttttggaacacgaccggcaagcatccggaactggtggaacattatgcgaaagcgtggcgcgcggtagctgaccgcttcgcggataatgatgcggttgtggcctatgacctgatgaatgagccgtttggtggctccctgcagggaccggcattcgaagcgggcccattagcagcaatgtaccagcgcactactgatgccatccgtcaggtggatcaggatacttgggtttgtgtggcaccgcaggccattggcgttaatcaaggtttaccatcgggcttaactaaaattgatgaccctcgcgccggtcaacaacgcattgcctattgcccgcatctgtacccgctgccattggacatcggcgacggccacgaaggacttgcgcgcactctgaccgatgtaaccattgatgcctggcgtgcgaacacggctcataccgcgcgcgtcttgggtgatgtgcctatcattctcggttcgttcggtttagataccacgctgcccggagcacgcgattacattgaacgtgtctatgggaccgcacgcgaaatgggtgcgggcgttagttattggtcgagtgatcccggcccgtggggcccgtatctgccggacggtacgcagaccttgttagtggataccttaaacaagccataccctcgtgcagtggcggggacccctaccgaatggagcagcacttcggatcgcctgcaattgaccattgaaccagatgccgctattaccgcgcctacagaaatctacctgcctgaggctggtttccccggggatgtgcatgtagaaggggcggatgtcgttggctgggatcgtcaatcgcgtcttttaaccgtacgcactcccgcggacagtggtaacgtcacagtgacagttacgcccgcagcgtgactcgag

SEQ ID NO: 32 - protein, 483 aa, Artificial Sequence
  Description of Artificial Sequencemutant endoglycoceramidase (His E351S) derived from Rhodococcus sp, strain M-177 endoglycoceramidase II, endoglycoceramide synthase
MGSSHHHHHHSSGLVPRGSHMGSSGSGSGSGTALTPSYLKDDDGRSLILRGFNTASSAKSAPDGMPQFTEADLAREYADMGTNFVRFLISWRSVEPAPGVYDQQYLDRVEDRVGWYAERGYKVMLDMHQDVYSGAITPEGNSGNGAGAIGNGAPAWATYMDGLPVEPQPRWELYYIQPGVMRAFDNFWNTTGKHPELVEHYAKAWRAVADRFADNDAVVAYDLMNEPFGGSLQGPAFEAGPLAAMYQRTTDAIRQVDQDTWVCVAPQAIGVNQGLPSGLTKIDDPRAGQQRIAYCPHLYPLPLDIGDGHEGLARTLTDVTIDAWRANTAHTARVLGDVPIILGSFGLDTTLPGARDYIERVYGTAREMGAGVSYWSSDPGPWGPYLPDGTQTLLVDTLNKPYPRAVAGTPTEWSSTSDRLQLTIEPDAAITAPTEIYLPEAGFPGDVHVEGADVVGWDRQSRLLTVRTPADSGNVTVTVTPAA

SEQ ID NO: 33 - protein, 490 aa, Artificial Sequence
  Description of Artificial Sequencemutant endoglycoceramidase derived from Rhodococcus sp, strain M-177 endoglycoceramidase II, endoglycoceramide synthase
MRRTRLVSLIVTGSLVFGGGVAAAQSSLAASGSGSGSGTALTPSYLKDDDGRSLILRGFNTASSAKSAPDGMPQFTEADLAREYADMGTNFVRFLISWRSVEPAPGVYDQQYLDRVEDRVGWYAERGYKVMLDMHQDVYSGAITPEGNSGNGAGAIGNGAPAWATYMDGLPVEPQPRWELYYIQPGVMRAFDNFWNTTGKHPELVEHYAKAWRAVADRFADNDAVVAYDLMNEPFGGSLQGPAFEAGPLAAMYQRTTDAIRQVDQDTWVCVAPQAIGVNQGLPSGLTKIDDPRAGQQRIAYCPHLYPLPLDIGDGHEGLARTLTDVTIDAWRANTAHTARVLGDVPIILGXFGLDTTLPGARDYIERVYGTAREMGAGVSYWSSDPGPWGPYLPDGTQTLLVDTLNKPYPRAVAGTPTEWSSTSDRLQLTIEPDAAITAPTEIYLPEAGFPGDVHVEGADVVGWDRQSRLLTVRTPADSGNVTVTVTPAA

SEQ ID NO: 34 - protein, 482 aa, Artificial Sequence
  Description of Artificial Sequencemutant endoglycoceramidase derived from Rhodococcus sp, strain C9 endoglycoceramidase, C9 EGCase, endoglycoceramide synthase
MRRTRIASLAVAGSLVLGAGVATAQSSLPATGSDSSEWSASAYLTDDAGRSLILRGFNTASSAKSTPDGMPIFTESDLDREHADMGTNFVRFLISWRSVEPEPGQYDQAYLDRVEQRVGWYAERGYKVMLDMHQDLYSGAITPDGKTGNGAPAWATYMDGLPVNERDSWELYYIEPGVIRAFDNFWNTTGKHPELVDHYVNAWKAVADRFADNETVVAYDLMNEPWGGSLQGPAFEAGPLTSMYQRTTDAIRQVDQDSWVCVAPQAVGVNQGIPSALGTIADPRQGARRIAYCPHLYPLPLDLGDGYSGFSKTLTDATIETWRTSIEHVADTVLEGAPVILGXFGLDTTLPGAQDYLDRVYTVARDMGAGVSYWSSDRGPWGPYLEDGTQTILVDTVNKPYPRAVAGMPVRWSSTSDRLDLTYRNDPAVTAPTEIYLPAAGFPGDIAVQGADVVGWDSQSRLLTVRSAPDAGEVTVTVTPAA

SEQ ID NO: 35 - protein, 500 aa, Artificial Sequence
  Description of Artificial Sequencemutant endoglycoceramidase derived from Propionibacterium acnes, strain KPA171202 endoglycoceramidase (EGCa)
MRRKSALGFVALSLFATGMGVAAATPATASPADTAAPVHVDASRWTTQGRWVTDTQHRVVITQGINEVAKSAPYAPDAVGFGEDDAAFLEAQGFTSVRLGVLWAGVEPRPGVYDDAYLARVERTVRILNAHGIASVLDFHQDMVNEKYQGEGWPAWAALDHGMPNIVKTGFPGNYFLNEAVKYSFDSFYDNTKASDGIGVADHYASAWRHVAEHFRNVPGVQGYDLFNEPFPGHRYTRCLTQLGCRAADARLSAVQQKTVDAIRSVDKATTVWYEPMQFFNIGVGTNVRLTGSNLGLSFHDYCTSQATLHSYVGCTAPDNRVFTNAEKHSRQTGSGLMLTXFGAITTPAVITSQMDLAARNRVGVQWWAYTAGDPTTAGPGTEQALVDDPARPPQGTNVESAKLTLIAVPHPDRVAGTPSAYHHDRSRRVFTMTWTAQRPDGSRAEESDETTVVVPAISAPHGYDVQASGAHVTSHPGDRVARLHLNQGSATAKVTITLR

SEQ ID NO: 36 - protein, 524 aa, Artificial Sequence
  Description of Artificial Sequencemutant endoglycoceramidase derived from Propionibacterium acnes, strain KPA171202 endoglycoceramidase (EGCb)
MYHHSWHSPDARRRGVTRWATTFIAALTAACMAQMPAQASPHTSDAAPHIATSKTITDAGPIGQSGRWYTDGQGRAILTAGVNMVSKRHPYSPEADGFDDADAAWLQKNGFDSVRLGVIWKGVEPKPGEYDDAYLASITRTVRTLRAHGIMTLLDAHQDMYNEKFEGEGAPDWAVLDKGAPNLLKVGFPANQVFNLGLIKAYDSFLDNAKGPGGVGLQDRYAAMWKHVAQVVGQEPGVMGYDIINEPWPGHHYPICYVAFGWCGRAMVSLDTLYEKVGRAITSVDPDGIVTYEPYSTWNMGLDSRPARPSSPKAAISWHVYCPMNAIFGSYVGCNLPDTRTFHNADQAAQFNNSASLLSXFGATKDPGTLMGVTSKARAHLVGWLYWTYNGNSDPTTQNAADEELVRHINRPGPVTDEQVDHTKLAILAVPHLRAAAGTPTSTTWDQSTRTYQATWTAKRVAGDGDFAAGSVSEIAVPAIHYPNGYKVEVKGAKVISKAGDTRLQVSSTGEGPVSVTITPAGQA

SEQ ID NO: 37 - protein, 503 aa, Artificial Sequence
  Description of Artificial Sequencemutant endoglycoceramidase derived from Cyanea nozakii jelly fish endoglycoceramidase (EGCase)
MAETQPLVFVLMSISAILTAGLPINDDASLLISVNPETQQLVDSLGRERFFHGTNVVVKHKPYHPSVEGYDNTSFSEVDMKILQDLGLNTIRLGMMLPGYVPTRGNYNETYLKIIQEIVSKAAKYGIYTLLDMHQDVMSAKFCVEGFPDWAVNTGNADNFPFPLEDKYPLNLQTGYPYPKDCAKHAWGDYYFTEAAAAAFQNFYNNTDGLLDAWADFWKKTAQGFKDYKSVIGYELINEPFAGDIYRDPSLMIPGVADERNLAPAYDVIHKAIRTVDEQHSIFFEGVTWDYFAAGFSKVPGGDAYRNRSVLSYHYYEPPDFNKKFQFEVRMEDLRRLKCGGFLTELLTVGDTAKDMSDMLXLFDICDQHKQSWMGWLYKSYGCYKQHLGCLTDSMHDETGHLRDIVLQNTTRTYPQAVAGHTIGYKFDRITKKFDLSFVVTADCRSTESIVYFNKDLHYSNGYDVTVFPKDSVTWKQVEKKIIINHSQKLSAGTTVTFSLVAK

SEQ ID NO: 38 - protein, 503 aa, Artificial Sequence
  Description of Artificial Sequencemutant endoglycoceramidase derived from Cyanea nozakii jelly fish endoglycoceramidase (EGCase-P)
MAETQPLVFVLMSISAILTAGLPINDDASLLISVNPETQQLVDSLGRERFFHGTNVVVKHKPYHPSVEGYDNTSFSEVDMKILQDLGLNTIRLGMMLPGYVPTRGNYNETYLKIIQEIVSKAAKYGIYTLLDMHQDVMSAKFCVEGFPDWAVNTGNADNFPFPLEDKYPLNPQTGYPYPKDCAKHAWGDYYFTEAAAAAFQNFYNNTDGLLDAWADFWKKTAQGFKDYKSVIGYELINEPFAGDIYRDPSLMIPGVADERNLAPAYDVIHKAIRTVDEQHSIFFEGVTWDYFAAGFSKVPGGDAYRNRSVLSYHYYEPPDFNKKFQFEVRMEDLRRLKCGGFLTELLTVGDTAKDMSDMLXLFDICDQHKQSWMGWLYKSYGCYKQHLGCLTDSMHDETGHLRDIVLQNTTRTYPQAVAGHTIGYKFDRITKKFDLSFVVTADCRSTESIVYFNKDLHYSNGYDVTVFPKDSVTWKQVEKKIIINHSQKLSAGTTVTFSLVAK

SEQ ID NO: 39 - protein, 517 aa, Artificial Sequence
  Description of Artificial Sequencemutant endoglycoceramidase derived from Hydra magnipapillata hydrozoan endoglycoceramidase (EGCase)
MISVALIILFLAKVISGKSDDFISVNPETNMLIDGYGRERFFHGTNVVVKHFPFHPETTGFNKDTFSEDDMKILQKFGLNSIRLGMMLPGYVPKREEYNETYIKVIQSIVTTAAKYGIYTLLDMHQDVFSPKFCVEGMPDWIVNTQGAKDFPMPLHKPFNLDPKTGYPYPEDCAKFSWADYYFTEAAGQAFQNLYDNVDGLRDEWAQFWKKTADVFKEEPSVIGYELINEPFCGNVFKHPTLLIPGVADYLNLQPTYDALQKAIRQVDEEHNIFFEGVTWDFFEVGFTEVPGGKQYQNRSVLSYHYYEPPDFSKKLNFEARLLDLKRLKCGGFLTEMFTVGTDFNSMFXMFDLCDKFKQSWHGWMYKSYGCIEQNLGCLNMSSPGKESIQIANTSRTYPQAVAGRTQSYAFDIKTKVFTLVYETVGSCKSGRTIVYFNKNLHYPNGYRYEINPNFKVTPSENEYFLYLDEVNKVPNTVVTFKLFPLSFTDSEDIHPVTVMGDKHLSENHNENEKKKK

SEQ ID NO: 40 - protein, 507 aa, Artificial Sequence
  Description of Artificial Sequencemutant endoglycoceramidase derived from Schistosoma japonicum blood fluke similar to endoglycoceramidase (EGCase)
MWSIFILTFLIWTSVQTKQIPLSKIHLNSDGLFTDSRGFIKLFRGFNNVHKHFPWYNVNSTNITQLEMFKNWGLNVVRLGVMWSGVKPTISIVNTTYLDVIENVIDLYADYGIYVILDMHQDVLSSLYGLYDGIPLWLIEKFKRPPHHLQYPWPYKKKPDFWVMSYLTYECANGAQQLYNNVSGAWNHWGEFWEIVARRFGGKSNVLGYELINEPPPGNFYTNPLRGLPGYAGRYNLQPVYDYLVKRIRKYDNSTLIFYEPVTYGVFTPVRSSGWLGTGFDRVPGAHRDKSAPSKSVLSYHYYCWILQTDAQNTTMPFWKKVICDRLLLPNVISNAIRATKSTGGGRFLTXFGLCGDDGNPRSVNTIECNNILNEADKHFESWTYWDSNLLDLSGNPIVTEVKSFIRPYPHSIRGVFRKQQFDHKTGDFHLSFIANTTKEQNNEKQTLIAEIYIPRSVHYPNGFSMSVKPDNLSTKMNENMMYVYLPSGVSNASVFVRIEIVRKSIE

SEQ ID NO: 41 - protein, 509 aa, Artificial Sequence
  Description of Artificial Sequencemutant endoglycoceramidase derived from Dictyostelium discoideum, strain AX4 slime mold putative endoglycoceramidase (EGCase)
MNKKKQIITTITLLSFINLFSIVNAIIKVNPANQFFIDQYNRVRLFHGVNVVYKIPPFHPSLEGFDPVTSFSSQDIENLVEWGFNAVRLGVMWPGVEPVKDEYNQTYLDVMSKLVSEMEDNEIYTLIDFHQDLLSRKYCGEGLPDWIVSNDTNDSFPSPVAHSYPKNNESYPSLDQCLNKDFGVYYFSEDVNREFQNLYDNVNGVQDKFIDYWRQVVNTFKSYDTVLGYEIINEPWGGDIYQNPEYLLKLGYADSKNLLPLYQAVNNAIRELDDQHCVYYEKALTDLFHSYFPSGTPGGVQYNDRQVLSYHIYCATDRDGNPRHEYVCDGEDDIFLVSAMKDLKQTGGGGFMTXFGAVSNGTNSIEMLNYLTGSADKYLQSWTYWQLKYYNDITTAGSTESLYLPNGELDIPKITALSRTYAQAIAGVPLSMSFNPANSDFSFSYNINTTITQPTQIYLNQDIYYPNGFTTNIITGTATVSIPQKNLIYILPNSNTINQSTITITILKK

SEQ ID NO: 42 - protein, 647 aa, Artificial Sequence
  Description of Artificial Sequencemutant endoglycoceramidase derived from Steptomyces avermitilis, strain MA-4680 putative endoglycoceramidase (EGCase)
MRKNAKLTHESEVLTFHRSARTVVDMSKLRARLLGVLVSLTGLLGATGAQPAAADSLPDSLWFDASASAAFTVQNGRFSDGLGREVVLRGYNVSGETKLEENSGLPFASVADARKSATALRTLGGGNSVRFLLSWAHAEPVRGQVDTAYLAAATAQMRAFLDAGIRVFPDFHQDLYSRYLFNSGSWYTGDGAPEWAVDAGDYPAESCGICLFWGQNITQNGAVTQASHDFWHNAYGVQDAFLATAQATMAYIQQNLSADEFNGVVGFDPYNEPHAGTYDSGETSRTWEQNVLWPFYKKFRARMDAAGWQTKPAFIEPNLFWNANIDFQKQEGGLLDAGTLGPRYVLNTHFYDQKAISGVLMWGKAADGQYATDFGKVRDRAAGAGTAAVVSXFGHPLSGSVSDKAPTVVKAMYQALDSRLPGSTWWSDPTGSGPVLSGAQWQWDIYNGRHHELENGNPDKVLTSGDAWNDEDLSAVSLNDSGTAVLRQDARLLDRLYPSATAGATVAFTYEDRSRDGSTTLTWNPVPSSLPNVSRLVGSGQYGLLVWRSNGSTAPTELHLPASFPAASTTVVSDLGTTSGLPAYTRTTPVGHAAEPGGTGSHRLLLTAADSGTVHYALVTNGATAPSAGLLSAARAELSSWAATKVG

SEQ ID NO: 43 - protein, 654 aa, Artificial Sequence
  Description of Artificial Sequencemutant endoglycoceramidase derived from Leptospira interrogans, serovar Copenhageni, strain Fiocruz L1-130 spirochete endoglycoceramidase (EGCase)
MEELFVKNGHFASKEGAIYQLRGVNLSGSAKLPLKPDGTTHFDQTTTFDNHKNVSFVGRPLKEDQAEEHFDRLRKWGFNFLRFLITWEAIEHKGPGKYDNEYIDYVERMVSLAAKKGFYLFIDPHQDVWSRFTGGDGAPGWTLEELGMNISKIRNSETAIVHHHQGKNYRRMSWPLNYQKYSCATMFSLFFGGKEFAPDTKIDGRNVQDFLQDHYIDSVLKIVRKLKKYKNVIGFDTLNEPSPGWIGKKNLGEFDGFGFGKVVKSSPFQEMYLSEGRAVSAAQAYMLGFWSLPFGKVRLNPEGVPLWERGHQCIWRNHGVWDYDPNGAPMMLKPEYFYKKNGRKYEFYSDFMYPFIKKFKERVQKLENRFHIFIESDPSKLELEWKEIPKKNQGSVINATHWYDISVLMLKRYLPWFGVHVFKQKPIFGKENIDNAYEETIRMIREMSEKKMGNCPTVIGXTGIPMDLNHRVAYLKNDYGVLEKALDRIMKAVEKNFVNLALWNYTPDHTHSLGDRWNEEDLSIYSQDTPSSYDEDGGRAVRAFSRPYPIRTKGFPVALTFDMERSLFKYAFRQEGDLFPETEIFIPEIHYKKGFEVLVNAGTYQYDFRSRVLKFKGEKGILDYGITVYPSKKSLSREQDRTKVVPKTQKRKTQ

SEQ ID NO: 44 - protein, 770 aa, Artificial Sequence
  Description of Artificial Sequencemutant endoglycoceramidase derived from Neurospora crassa, strain OR74A fungus putative endoglycoceramidase (EGCase)
MAGFRLTIENGSFRDVHGRQITLRGINVAGDAKYPNKPEQPSHVGENFFDGDNVKFTGRPFPKEEAHLHFSRLKRFGYNTIRYVFTWEAIEAAGPGIYDEEWIQHTIDVLRVAKRYGFYIFMDPHQDVWSRFSGGSGAPMWTLYAAGLNPQSFAATEAAIVHNVYPEPHNFPKMIWSTNYYRLAAATMFTLFFAGRDFAPKCIIDGVNIQDYLQDHFLRACAHLAQRIHEAGDIENDVVFGWESLNEPNKGMIAYEDISVIPKEQNLKKGTCPTIWQTILTGSGRAVEVDTWDMGGMGPYKVGRALIDPSGEQAWLPADYDESRYGYKRDPGWKLGQCIWAQHGVWDPATDSLLKKDYFGKHPATGEHVDYPYFSNRYFMDFFRKYRDTIRSIHPNAIILLQGPTMELPPKIIGTPDGDDPLLVYAPHWYDGITLMTKKWNRVWNVDVIGILRGKYWSPAFGIKIGETAIRNCFKNQHATMRQEGLDYIGNHPCVMTXFGIPYDMDDKNAYKTGDYSSQSAAMDANHYGVEGAGLEGYTLWLYMTKNDHELGDQWNGEDLSIFSVDDKLLPESPVPKSHSRDGSSSSIATPTGTKDDDLDDDSSVTPANIKRTLTNPSISSVSTQRQPELTNSPGYRAAEAYVRPAPIATAGTVKKYGFDLRSCQFHVTIQAPEAAKPDTPTVVFLPDYHFPKDACQVEVSSGKWEIRSDEEETTPLQKLRWWHGEGEQTLRVTGVVKQVNGNSSEGAEVGYYDQVFNQAKGFLDACVIM

SEQ ID NO: 45 - DNA, 31 bp, Artificial Sequence
  Description of Artificial Sequence5′ PCR primer 5′ Copt
aattcgattggatcccatatgagcggaagcg

SEQ ID NO: 46 - DNA, 39 bp, Artificial Sequence
  Description of Artificial Sequence3′ PCR primer 3′ Asp PstI
tcgattctgcagggagccaccaaacgggtcattcatcag

SEQ ID NO: 47 - DNA, 39 bp, Artificial Sequence
  Description of Artificial Sequence3′ PCR primer 3′ Gln PstI
tcgattctgcagggagccaccaaacggctgattcatcag

SEQ ID NO: 48 - DNA, 39 bp, Artificial Sequence
  Description of Artificial Sequence3′ PCR primer 3′ Ala PstI-11-1
cggtccctgcagggagccaccaaacggcgcattcatcag

SEQ ID NO: 49 - DNA, 39 bp, Artificial Sequence
  Description of Artificial Sequence3′ PCR primer 3′ Gly PstI-11-1
cggtccctgcagggagccaccaaacggcccattcatcag

SEQ ID NO: 50 - DNA, 39 bp, Artificial Sequence
  Description of Artificial Sequence3′ PCR primer 3′ Ser PstI-11-1
cggtccctgcagggagccaccaaacggcgaattcatcag

SEQ ID NO: 51 - protein, 6 aa, Artificial Sequence
  Description of Artificial Sequencesix His tag
HHHHHH